SEQUENCE LISTING (17 sequences)

SEQ ID NO: 1 - DNA, 35 bp, Artificial Sequence
  Synthesized
ttgacagctagctcagtcctaggtataatgctagc

SEQ ID NO: 2 - DNA, 35 bp, Artificial Sequence
  Synthesized
ttgacagctagctcagtcctaggtactgtgctagc

SEQ ID NO: 3 - DNA, 35 bp, Artificial Sequence
  Synthesized
tttacagctagctcagtcctaggtattatgctagc

SEQ ID NO: 4 - DNA, 35 bp, Artificial Sequence
  Synthesized
ctgacagctagctcagtcctaggtataatgctagc

SEQ ID NO: 5 - DNA, 35 bp, Artificial Sequence
  Synthesized
tttacggctagctcagtcctaggtacaatgctagc

SEQ ID NO: 6 - DNA, 35 bp, Artificial Sequence
  Synthesized
ttgacagctagctcagtcctagggactatgctagc

SEQ ID NO: 7 - DNA, 31 bp, Escherichia coli
tcgccagtctggcctgaacatgatataaaat

SEQ ID NO: 8 - DNA, 178 bp, Escherichia coli
aaccactatcaatatattcatgtcgaaaatttgtttatctaacgagtaagcaaggcggattgacggatcatccgggtcgctataaggtaaggatggtcttaacactgaatccttacggctgggttagccccgcgcacgtagttcgcaggacgcgggtgacgtaacggcacaagaaacg

SEQ ID NO: 9 - DNA, 170 bp, Escherichia coli
atgcgggttgatgtaaaactttgttcgcccctggagaaagcctcgtgtatactcctcacccttataaaagtccctttcaaaaaaggccgcggtgctttacaaagcagcagcaattgcagtaaaattccgcaccattttgaaataagctggcgttgatgccagcggcaaac

SEQ ID NO: 10 - DNA, 35 bp, Artificial Sequence
  Synthesized
ttgacagctagctcagtcctaggtacagtgctagc

SEQ ID NO: 11 - DNA, 35 bp, Artificial Sequence
  Synthesized
ttgacagctagctcagtcctaggtacaatgctagc

SEQ ID NO: 12 - DNA, 29 bp, Escherichia coli
ctaatgagcgggcttttttttgaacaaaa

SEQ ID NO: 13 - DNA, 38 bp, Artificial Sequence
  Synthesized
aaaaaaaaaaaaccccgcttcggcggggtttttttttt

SEQ ID NO: 14 - DNA, 44 bp, Escherichia coli
ataaaacgaaaggctcagtcgaaagactgggcctttcgttttat

SEQ ID NO: 15 - DNA, 28 bp, Escherichia coli
agaaggccatcctgacggatggcctttt

SEQ ID NO: 16 - DNA, 3663 bp, Chloroflexus aurantiacus
atgtctggtactggtcgactggcaggtaaaattgcactgatcactggcggtgctggcaatattggttccgagctgacccgccgtttcctggccgagggcgcgaccgtcatcatctctggtcgtaaccgcgccaaactgaccgcactggcagagcgtatgcaagcagaggctggtgtgccggctaagcgtattgatctggaagtcatggacggtagcgatccagtcgctgtgcgcgctggtattgaagcgattgtggctcgccatggtcagattgatattctggttaacaatgctggttccgcgggtgcacagcgtcgcctggccgaaattccgctgaccgaggccgaactgggtccgggcgctgaggaaactctgcacgcgtccatcgcaaatctgctgggtatgggctggcacctgatgcgcattgcggctccacacatgccggttggttccgcagttatcaacgtttccaccattttcagccgcgctgaatactatggtcgtattccgtacgttacgccgaaagccgctctgaacgcgctgtcccagctggcggcacgcgagctgggcgctcgtggtattcgtgtcaacactatcttcccgggtccgatcgagtccgaccgtatccgtactgtctttcaacgcatggaccagctgaaaggtcgccctgagggcgacaccgctcatcacttcctgaacaccatgcgtctgtgccgtgcgaacgatcagggcgctctggaacgtcgcttcccgtccgtgggtgatgtggcggacgcggctgtgttcctggcgtctgccgaatctgcggcactgtctggtgagactattgaagtgactcacggcatggagctgccggcgtgctctgagactagcctgctggctcgtacggatctgcgcaccatcgacgctagcggtcgcaccaccctgatctgtgcgggcgaccagattgaagaagtgatggcgctgaccggtatgctgcgtacctgcggctctgaagttattatcggcttccgctccgcagcagcgctggcccagtttgaacaggcggtcaacgaaagccgtcgtctggcaggtgctgattttactccaccaatcgccctgccgctggacccgcgtgatccggcaactatcgatgctgtgtttgactggggcgcaggtgaaaacaccggcggcatccacgctgctgttatcctgccggcaacctctcatgagccagccccttgtgtgatcgaggttgatgacgagcgtgttctgaacttcctggctgacgagattaccggcacgatcgttatcgcgtctcgtctggctcgctactggcagtctcagcgcctgacccctggtgcacgtgcccgtggccctcgtgttatctttctgtccaatggcgcggatcagaacggtaacgtctatggccgtatccaatctgctgctatcggccaactgattcgtgtttggcgtcacgaagctgagctggattaccagcgtgcatccgcagctggcgatcacgtgctgccgcctgtctgggccaaccaaatcgttcgcttcgctaaccgctctctggagggcctggagtttgcatgcgcctggacggcccagctgctgcactctcagcgtcatatcaatgaaatcactctgaacatccctgcgaacattagcgctactaccggtgctcgttctgcttctgtcggttgggcggaatctctgatcggtctgcacctgggcaaagtggcgctgatcaccggtggctctgcgggcatcggtggccagatcggccgtctgctggcgctgtctggcgcacgcgtgatgctggctgcacgtgaccgtcacaaactggagcagatgcaggcaatgattcagagcgagctggcggaagtcggctacactgacgttgaagaccgcgtccacatcgctccgggctgcgacgtgtcttctgaggctcagctggctgatctggtcgaacgcaccctgtctgcattcggtacggtggactacctgatcaacaatgcgggcattgccggtgtcgaggagatggtgatcgacatgccagtcgaaggttggcgccacacgctgttcgcgaatctgatcagcaattacagcctgatgcgtaaactggcgccgctgatgaaaaagcagggttctggctacatcctgaacgtttcttcctacttcggcggcgaaaaggatgcggccatcccatatccgaaccgcgcagattacgcggtttctaaagccggccagcgtgcgatggcagaagtgttcgcccgcttcctgggtccggagatccagattaacgcgatcgcaccgggtccggttgaaggtgatcgcctgcgtggtacgggtgaacgtccgggcctgttcgcacgtcgtgcgcgtctgatcctggaaaacaagcgcctgaatgagctgcacgcggccctgattgcagccgcgcgtaccgacgaacgttctatgcacgagctggtggagctgctgctgccgaacgatgtggctgccctggaacagaatccagcagcaccgaccgcgctgcgcgaactggcccgtcgttttcgttccgaaggcgatccggctgcatcctcctccagcgcactgctgaaccgttctatcgcggcgaagctgctggcacgcctgcacaatggtggttacgtcctgccagccgacatcttcgcaaacctgcctaacccaccggatccattctttacccgcgctcagatcgaccgtgaagcgcgtaaagttcgtgatggtatcatgggcatgctgtatctgcagcgtatgccgacggagttcgatgtcgcgatggcaaccgtctattacctggccgaccgcaacgtgagcggcgaaaccttccacccatccggtggcctgcgctatgaacgtacgccgaccggtggtgagctgttcggcctgccgagcccggaacgcctggcagaactggttggctccaccgtgtacctgatcggtgaacacctgacggagcacctgaacctgctggcccgtgcgtatctggagcgttatggcgcacgtcaagttgttatgatcgtggaaaccgaaacgggtgccgaaactatgcgtcgtctgctgcacgaccatgtcgaagccggccgcctgatgacgatcgtggctggtgaccagatcgaagcagccatcgatcaggcaattacgcgttatggtcgtccgggtcctgttgtttgcactccattccgcccgctgccaactgtgcctctggtcggtcgcaaggactccgattggagcacggtcctgtctgaagctgagttcgcggaactgtgcgagcatcagctgactcaccacttccgtgttgctcgcaagatcgcactgtccgatggcgccagcctggcgctggtcaccccagagactaccgcaacttctaccactgaacaattcgctctggcaaacttcattaaaactacgctgcacgctttcaccgcgaccatcggcgttgagtccgaacgtacggcgcagcgtatcctgatcaatcaggtggatctgactcgtcgtgcgcgcgccgaagaaccgcgcgatccgcacgaacgccagcaggaactggagcgcttcattgaagcagtcctgctggtcactgcgcctctgccaccggaagcggacacgcgctatgccggtcgcatccatcgcggccgtgccatcactgtctga

SEQ ID NO: 17 - protein, 1202 aa, Chloroflexus aurantiacus
MSGTGRLAGKIALITGGAGNIGSELTRRFLAEGATVIISGRNRAKLTAERMQAEAGVPAKRIDLEVMDGSDPVAVRAGIEAIVARHGQIDILVNNAGSAGAQRRLAEIPLTEAELGPGAEETLHASIANLLGMGWHLMRIAAPHMPVGSAVINVSTIFSRAEYYGRIPYVTPKAALNALSQLAARELGRIRVNTIFPGPIESDRIRTVFQRMDQLKGRPEGDTAHHFLNTMRLCRANDQGALERRFPSVGDVADAAVFLASAESAALSGETIEVTHGMELPACSETSLLARTDLRTIDASGRTTLICAGDQIEEVMALTGMLRTCGSEVIIGFRSAAAQFEQAVNESRRLAGADFTPPIALPLDPRDPATIDAVFDWGAGENTGGIHAAVILPATSHEPAPCVIEVDDERVLNFLADEITGTIVIASRLARYWQSQRLTPGARRPRVIFLSNGADQNGNVYGRIQSAAIGQLIRVWRHEAELDYQRASAAGDHVLPPVWANQIVRFANRSLEGLEFACAWTAQLLHSQRHINEITLNIPANISATTGARSASVGWAESLIGLHLGKVITGGSAGIGGQIGRLLALSGARVMLAARDRHKLEQMQAMIQSELAEVGYTDVEDRVHIAPGCDVSSEAQLADLVERTLSAFGTVDYLINNAGIAGVEEMVIDMPVEGWRHTLFANLISNYSLMRKLAPLMKKQGSGYILNVSSYFGGEKDAAIPYPNRADYAVSKAGQRAMAEVFARFLGPEIQINAIAPGPVEGDRLRGTGERPGLFARRARLINKRLNELHAALIAAARTDERSMHELVELLLPNDVAALEQNPAAPTALRELARRFRSEGDPAASSSSALLNRSIAAKLLARLHNGGYVLPADIFANLPNPPDPFFTRAQIDREARKVRDGIMGMLYLQRMPTEFDVAMATVYYLADRNVSGETFHPSGGLRYERTPTGGELFGLPSPERLAELVGSTVYLIGEHLTEHLNLLARAYLERYGARQVVMIVETETGAETMRRLLHDHVEAGRLMTIVAGDQIEAAIDQAITRYGRPGPVVCTPFRPLPTVPLVGRKDSDWSTVLSEAEFAELCEHQLTHHFRVARKIALSDGASLALVTPETTATSTTEQFANFIKTTLHAFTATIGVESTAQRILINQVDLTRRARAEEPRDFRQQELERFIEAVLLVTAPLPPEADTRYAGRIHRGRAITV

(19) United States
(12) Patent Application Publication
(10) Pub. No.: US 2017/0016035 A1
(43) Pub. Date: Jan. 19, 2017

(54) GENETICALLY ENGINEERED METHYLOTROPHS FOR THE PRODUCTION OF PHA BIOPOLYMERS AND C3, C4, AND C5 BIOCHEMICALS FROM METHANOL OR METHANE AS SOLE CARBON FEEDSTOCK

(71) Applicant: METABOLIX, INC., Cambridge, MA (US)

(72) Inventors: Thomas M. Ramseier, Newton, MA (US); Dong-eun Chang, Cambridge, MA (US); Jian-rong Gao, Cambridge, MA (US); William R. Farmer, Concord, MA (US); Oliver P. Peoples, Arlington, MA (US)

(21) Appl. No.: 14/901,540

(22) PCT Filed: Jun. 27, 2014
(86) PCT No.: PCT/US2014/044706

§ 371 Date: Dec. 28, 2015

Related U.S. Application Data
(60) Provisional application No. 61/841,275, filed on Jun. 28, 2013.
(60) Provisional application No. 61/893,311, filed on Oct. 21, 2013.

(51) Int. Cl. C12P 7/62; C12P 7/18

(52) U.S. Cl. CPC C12P 7/625; C12P 7/18

(57) ABSTRACT

Methods and genetically engineered hosts for the production of 3-carbon, 4-carbon and 5-carbon products, polymers and copolymers in methylotrophic bacteria are described herein.

### RELATED APPLICATIONS

This application claims the benefit of U.S. Provisional Application No. 61/811,275, filed on Jun. 28, 2013 and claims the benefit of U.S. Provisional Application No. 61/893,311, filed on Oct. 21, 2013. The entire teachings of the above application(s) are incorporated herein by reference.

### INCORPORATION BY REFERENCE OF MATERIAL IN ASCII TEXT FILE

This application incorporates by reference the Sequence Listing contained in the following ASCII text file being submitted concurrently herewith:
a) File name: 46141014002SEQUENCELISTING.txt; created May 8, 2014, 18 KB in size.

### BACKGROUND

With the recent ability to access the vast amount of natural gas trapped in shale rock formations using technologies such as hydraulic fracturing and horizontal drilling, the price for American natural gas has decreased to a fraction of that in earlier years. Biobased, “green” natural gas is produced from renewable resources that are formed by the breakdown of organic matter such as manure, sewage, municipal waste, green waste, plant material, and crops in the absence of oxygen. Natural gas consists primarily of methane (CH₄). Methane is used as an energy source for heating, cooking, and electricity generation. It is also the C1 carbon source for the commercial production of methanol (CH₃OH, often abbreviated MeOH). Methanol is an important chemical building block used for many organic intermediates and downstream processes including esterification, ammoniation, methylation, and polymerization. The primary chemical intermediates produced from methanol include formaldehyde, acetic acid, methylamines, methyl methacrylate (MMA), dimethyl terephthalate (DMT) and methyl tertiary butyl ether (MTBE). It is also used as antifreeze, solvent, fuel, a denaturant for ethanol, and to produce biodiesel via transesterification reaction. Methanol is produced in a three stage process that includes (1) reforming where methane is combined with steam under heat to produce synthesis gas, a mixture of hydrogen (H₂), carbon monoxide (CO) and carbon dioxide (CO₂), (2) compression conversion where the synthesis gas is pressurized and converted to methanol, and (3) distillation where the liquid mixture is heated to separate the components and the resulting vapor is cooled and condensed to produce pure methanol. Methanol can consequently be produced very cost-effectively from methane. Biobased, “green” methanol (bio-methanol) can also be produced from renewable raw materials such as glycerol on a large industrial scale as shown by BioMCN at the world wide web at biomcn.eu.

Both methane and methanol can also be an inexpensive alternative carbon feedstock utilized by methylotrophic microorganisms for the production of valuable industrial chemicals. Methylotrophs are capable of growth on C1-compounds (single carbon-containing compounds) as their sole source of carbon and energy and thus are able to make every carbon-carbon bond de novo. C1 substrates that are used for methylotrophic growth include not only methane and methanol, but also methylamine (CH₃NH₂), formaldehyde (HCHO), formate (HCOOH), formamide (HCONH₂), and carbon monoxide (CO). Examples for use of methane as sole carbon feedstock include the wild-type methanotrophic bacterium Methylococcus capsulatus (Bath) that was used by Norferm Danmark A/S to produce BioProtein, a bacterial single cell protein (SCP) product serving as a protein source in feedstuff (Bothe et al., Appl. Microbiol. Biotechnol. 59:33-39 (2002)), and production of poly-3-hydroxybutyrate (PHB) using Methylocystis hirsute (Rahnama et al., Biochem. Engineer. J. 65:51-56 (2012)) or Methylocystis sp. GB 25 wild-type strains (Wendlandt et al., J. Biotech. 86:127-133 (2001)). The obligate methanotrophic Methylomonas sp. strain 16a was genetically engineered to produce astaxanthin from methane (Ye et al., J. Ind. Microbiol. Biotechnol. 34:289-299 (2007)). Industrial-scale processes using methanol as sole carbon feedstock were established by Imperial Chemical Industries (ICI) in the 1970s and 80s with the aim of providing large amounts of SCP (soluble carbohydrate polymer) for human and animal feed.

Production of poly-3-hydroxybutyrate (PHB) has also been accomplished using methanol as the sole carbon source in wild-type methylotrophs, where PHB concentrations of up to 130 g/L were obtained and PHB accumulated up to 60% of the total biomass (Kim et al., Biotechnol. Lett. 18:25-30 (1996); Zhao et al., Appl. Biochem. Biotechnol. 39-40:191-199 (1993)). Production of the copolymer poly(3-hydroxybutyrate-co-3-hydroxyvalerate) in wild-type methylotrophs was accomplished when a mixture of methanol and n-amyl-alcohol, valeric acid, or propionic acid was fed to the fermentation medium (Haywood et al., Biotechnol. Lett. 11(7):471-476 (1989); Bourque et al., Appl. Microbiol. Biotechnol. 37(1):7-12 (1992); Ueda et al., Appl. Environ. Microbiol. 58(11):3574-3579 (1992)). Genetic engineering of Methylobacterium extorquens to express the phaC1 or phaC2 genes encoding the PHA synthase 1 or 2, respectively, from Pseudomonas fluorescens enabled production of functionalized PHA copolymer when n-alkenoic acids were co-fed with methanol (Höfer et al., Microb. Cell Fact. 9:70 (2010), PMID: 20846434, DOI: 10.1186/1475-2859-9-70; Höfer et al., Biochem. Eng. J. 54:26-33 (2011), Höfer et al., Bioengineered Bugs 2(2):71-79 (2011)).

Previous work has shown that it is possible to produce a very limited range of PHA materials in microorganisms using C1 compounds as the sole carbon feedstock. There is a need therefore to engineer methylotrophic microorganisms to enable the production of a wider variety of PHA biopolymers as well as C3, C4, and C5 biochemicals from methanol or methane as the sole carbon feedstock.

### SUMMARY OF THE INVENTION

The invention generally relates to methods of increasing the production of a 3-carbon (C3) product or polymer of 3-carbon monomers, 4-carbon (C4) product or a polymer of 4-carbon monomers, or 5-carbon (C5) product or polymer of 5-carbon monomers or copolymers thereof from methanol or methane in methylotrophic bacteria. Metabolic pathways in bacteria are genetically engineered by providing one or more genes that are stably expressed that encodes an enzyme with an activity catalyzing the methanol or methane to produce the carbon products, polymer or copolymers, wherein microorganism growth is improved and the carbon flux from the renewable feedstock is increased.

In certain embodiments of any of the aspects of the invention, the pathway is a malonyl CoA metabolic pathway, an acetyl-CoA pathway, a 3-hydroxypropioate CoA pathway, a 4-hydroxybutyrate-CoA pathway, a 5-hydroxyvalerate-pathway, a succinate semialdehyde dehydrogenase pathway optionally including an alpha-ketoglutarate decarboxylase pathway, an alpha-ketoglutarate pathway, a lysine pathway,

The invention also pertains to increasing the amount of poly 3 hydroxypropionate (P3HP) homopolymer, P(3HB-co-3HP) copolymer, and 1,3-propanediol (PDO) in methylotrophic bacteria. In other aspects, the invention pertains to increasing the amount of poly-4-hydroxybutyrate (P4HB) homopolymer, P(3HB-co-4HB) copolymer, and 1,4-butanediol (BDO) in methylotrophic bacteria. Exemplary pathways for production of these products are provided in FIGS. 1-3. It is understood that additional enzymatic changes that contribute to this pathway can also be introduced or suppressed for a desired production of carbon product, polymer or co-polymers.

In a first aspect, the invention pertains to a method of increasing the production of a 3-carbon (C3) product, a 4-carbon (C4) product or a 5-carbon (C5) product, a polymer of 3-carbon monomers, a polymer of 4-carbon monomers or a polymer of 5-carbon monomers or copolymer combinations thereof from a renewable feedstock of methane or methanol, by providing a genetically modified methylotroph organism having a modified or metabolic C3, C4 or C5 pathway or incorporating a modified metabolic C3, C4 or C5 pathway, and providing one or more genes that are stably expressed that encodes one or more enzymes of the carbon pathway, wherein the production of the carbon product, polymer or copolymer is improved compared to a wild type organism. In a first embodiment of the first aspect, the wild type methylotroph naturally produces polyhydroxybutyrate. In a second embodiment of the first aspect, the wild type methylotroph is genetically modified to produce polyhydroxybutyrate. In a third embodiment of the first aspect or any of the other embodiments, the product, polymer or copolymer is a 3-carbon product, polymer or copolymer and the methylotroph has a modified metabolic C3 pathway; the product, polymer or copolymer is a 4-carbon product, polymer or copolymer and the methylotroph has a modified metabolic C4 pathway; or the product, polymer or copolymer is a 5-carbon product, polymer or copolymer and the methylotroph has a modified metabolic C5 pathway.

In a fourth embodiment, of the first aspect or any other embodiment, the feedstock is methanol or methane.

In a fifth embodiment of the first aspect, the product is poly-3-hydroxypropionate, the feedstock is methanol and the modified genetic pathway is a malonyl-CoA reductase metabolic pathway and the one or more genes that are stably expressed encode one or more enzymes or mutants and homologues thereof are selected from: acetyl-CoA carboxylase, malonyl-CoA reductase (3-hydroxypropionate-forming), malonyl-CoA reductase (malonate semialdehyde-forming), malonic semialdehyde reductase, CoA transferase, CoA ligase, and polyhydroxyalkanoate synthase, wherein the expression increases the production of poly-3-hydroxypropionate, wherein the expression increases the production of poly-3-hydroxypropionate. For example, the one or more genes that are stably expressed encode one or more enzyme are selected from: an acetyl-CoA carboxylase subunits from E. coli or mutants and homologues thereof; a malonyl-CoA reductase (3-hydroxypropionate-forming) from Chloroflexus aurantiacus or mutants and homologues thereof; malonyl-CoA reductase (malonate semialdehyde-forming) from Sulfolobus tokodaii str. 7 or mutants and homologues thereof; malonic semialdehyde reductase from Sulfolobus tokodaii str. 7 or mutants and homologues thereof; CoA transferase from Clostridium kluyveri DSM 555, or mutants and homologues thereof; CoA ligase from Pseudomonas putida or mutants and homologues thereof; and polyhydroxyalkanoate synthase from a fusion protein of Pseudomonas putida and Ralstonia eutropha JMP134 or mutants and homologues thereof wherein the expression increases the production of poly-3-hydroxypropionate. In a certain aspect of the fifth embodiment, the modified organism is Methylophilus methylotrophus.

In a sixth embodiment of the first aspect, the product is poly-3-hydroxypropionate, the feedstock is methanol and the modified genetic pathway is a dihydroxyacetone-phosphate metabolic pathway. The one or more genes that are stably expressed encode one or more enzymes or mutants and homologues thereof are selected from: glycerol-3-phosphate dehydrogenase (NAD+); glycerol-3-phosphate dehydrogenase (NADP+); glycerol-3-phosphatase; glycerol dehydratase; glycerol dehydratase reactivating enzyme; CoA transferase, CoA ligase, aldehyde dehydrogenase; alcohol dehydrogenase; CoA-acylating 3-hydroxypropionaldehyde dehydrogenase; and polyhydroxyalkanoate synthase, wherein the expression increases the production of poly-3-hydroxypropionate. For example, the one or more genes that are stably expressed encode one or more enzyme are selected from glycerol-3-phosphate dehydrogenase (NAD+) from Saccharomyces cerevisiae S288c or mutants and homologues thereof; glycerol-3-phosphate dehydrogenase (NADP+) from Rickettsia prowazekii (strain Madrid E) or mutants and homologues thereof; glycerol-3-phosphatase from Saccharomyces cerevisiae S288c or mutants and homologues thereof; glycerol dehydratase small, medium and large subunits from Klebsiella pneumonia or mutants and homologues thereof; glycerol dehydratase reactivating enzyme (Chain A and Chain B) from Klebsiella pneumonia or mutants and homologues thereof; aldehyde dehydrogenase/alcohol dehydrogenase from E. coli str. K-12 substr. MG1655; or mutants and homologues thereof; CoA transferase from Clostridium kluyveri DSM 555, or mutants and homologues thereof; CoA ligase from Pseudomonas putida or mutants and homologues thereof; 3-hydroxy-propionaldehyde dehydrogenase (gamma-Glu-gamma-aminobutyraldehyde dehydrogenase, NAD(P)H-dependent) from E. coli str. K-12 substr. MG1655 or mutants and homologues thereof; CoA-acylating 3-hydroxypropionaldehyde dehydrogenase from Salmonella enterica subsp. enterica serovar Typhimurium str. LT2 or mutants and homologues thereof; and polyhydroxyalkanoate synthase from a fusion protein of Pseudomonas putida and Ralstonia eutropha JMP134 or mutants and homologues thereof; wherein the expression increases the production of poly-3-hydroxypropionate.

In a certain aspect of the sixth embodiment, the organism is Methylophilus methylotrophus.

In the seventh embodiment of first aspect, the product is poly-3-hydroxybutyrate-co-3-hydroxyproprionate copolymer and the feedstock is methanol and the modified genetic pathway is a malonyl-CoA reductase metabolic pathway. The one or more genes that are stably expressed encode one or more enzyme are selected from acetyl-CoA acetyltransferase; acetoacetyl-CoA reductase; acetyl-CoA carboxylase, malonyl-CoA reductase (3-hydroxypropionate-forming), malonyl-CoA reductase (malonate semialdehyde-forming), malonic semialdehyde reductase, CoA transferase, CoA ligase, and polyhydroxyalkanoate synthase, wherein the expression increases the production of poly-3-hydroxybutyrate-co-3-hydroxyproprionate copolymer.

For example, the one or more genes that are stably expressed encode one or more enzyme are selected from acetyl-CoA acetyltransferase from Zoogloca rumigera or mutants and homologues thereof; acetoacetyl-CoA reductase from Zoogloea ramigera or mutants and homologues thereof; an acetyl-CoA carboxylase subunits from E. coli or mutants and homologues thereof; a malonyl-CoA reductase (3-hydroxypropionate-forming) from Chloroflexus aurantiacus or mutants and homologues thereof; malonyl-CoA reductase (malonate semialdehyde-forming) from Sulfolobus tokodaii str. 7 or mutants and homologues thereof; malonic semialdehyde reductase from Sulfolobus tokodaii str. 7 or mutants and homologues thereof; CoA transferase from Clostridium kluyveri DSM 555, or mutants and homologues thereof. CoA ligase from Pseudomonas putida or mutants and homologues thereof; and polyhydroxyalkanoate synthase from a fusion protein of Pseudomonas putida and Ralstonia eutropha JMP134 or mutants and homologues thereof; wherein the expression increases the production of poly-3-hydroxybutyrate-co-3-hydroxyproprionate copolymer. In a certain embodiment of the seventh embodiment, the organism is methylophilus methylotrophus or the organism is Methylobacterium extorquens with one or more of the following genes deleted: phaC1, phaC2, depA and depB.

In the eighth embodiment of the first aspect, the product is poly-3-hydroxybutyrate-co-3-hydroxyproprionate copolymer, the feedstock is methanol and the modified genetic pathway is a dihydroxyacetone-phosphate metabolic pathway. The one or more genes that are stably expressed encode one or more enzymes are are selected from: glycerol-3-phosphate dehydrogenase (NAD+); glycerol-3-phosphate dehydrogenase (NADP+); glycerol-3-phosphatase; glycerol dehydratase; glycerol dehydratase reactivating enzyme; aldehyde dehydrogenase; alcohol dehydrogenase; and aldehyde reductase, wherein the expression increases the production of poly-3-hydroxybutyrate-co-3-hydroxyproprionate copolymer. For example the one or more genes that are stably expressed encode one or more enzyme are selected from glycerol-3-phosphate dehydrogenase (NAD+) from Saccharomyces cerevisiae S288c or mutants and homologues thereof; glycerol-3-phosphate dehydrogenase (NADP+) from Rickettsia prowazekii (strain Madrid E) or mutants and homologues thereof; glycerol-3-phosphatase from Saccharomyces cerevisiae S288c or mutants and homologues thereof; glycerol dehydratase small, medium and large subunits from Klebsiella pneumonia or mutants and homologues thereof; glycerol dehydratase reactivating enzyme (Chain A and Chain B) from Klebsiella pneumonia or mutants and homologues thereof; 3-hydroxy-propionaldehyde dehydrogenase (gamma-Glu-gamma-aminobutyraldehyde dehydrogenase, NAD(P)H-dependent) from E. coli str. K-12 substr. MG1655 or mutants and homologues thereof; and aldehyde reductase (succinic semialdehyde reductase) from E. coli K-12 or mutants and homologues thereof; wherein the expression increases the production of poly-3-hydroxybutyrate-co-3-hydroxyproprionate copolymer.

In a certain embodiment of the eighth embodiment the organism is Methylophilus methylotrophus or Methylobacterium extorquens with one or more of the following genes deleted: phaA, phaB, phaC1, phaC2, depA and depB.

In a ninth embodiment of the first aspect, the product is 1,3-propanediol, the feedstock is methanol and the modified genetic pathway is a malonyl-CoA reductase metabolic pathway.

The one or more genes that are stably expressed encode one or more enzymes are selected from: acetyl-CoA carboxylase, malonyl-CoA reductase (3-hydroxypropionate-forming), malonyl-CoA reductase (malonate semialdehyde-forming), malonic semialdehyde reductase, aldehyde dehydrogenase/alcohol dehydrogenase; and aldehyde reductase wherein the expression increases the production of 1,3-propanediol. For example, the one or more genes that are stably expressed encode one or more enzymes are selected from: acetyl-CoA carboxylase subunits from E. coli or mutants and homologues thereof; malonyl-CoA reductase 3-hydroxypropionate-forming from Chloroflexus aurantiacus or mutants and homologues thereof; malonyl-CoA reductase (malonate semialdehyde forming from Sulfolobus tokodaii str. 7 or mutants and homologues thereof; malonic semialdehyde reductase from Sulfolobus tokodaii str. 7 or mutants and homologues thereof, aldehyde dehydrogenase/alcohol dehydrogenase 3-hydroxy-propionaldehyde dehydrogenase (gamma-Glu-gamma-aminobutyraldehyde dehydrogenase, NAD(P)H-dependent) from E. coli str. K-12 substr. MG1655 or mutants and homologues thereof; and succinic aldehyde reductase from E. coli K-12 or mutants and homologues thereof; wherein the expression increases the production of 1,3-propanediol. In a certain embodiment of the ninth embodiment, organism is Methylophilus methylotrophus.

In the tenth embodiment of the first aspect, the product is 1,3-propanediol, the feedstock is methanol and the modified genetic pathway is a dihydroxyacetone-phosphate metabolic pathway. The one or more genes that are stably expressed encode one or more enzymes are selected from: acetyl-CoA carboxylase, malonyl-CoA reductase (3-hydroxypropionate-forming), malonyl-CoA reductase (malonate semialdehyde-forming), malonic semialdehyde reductase, aldehyde dehydrogenase/alcohol dehydrogenase; and aldehyde reductase wherein the expression increases the production of 1,3-propanediol. Fore example, the one or more genes that are stably expressed encode one or more enzymes are selected from: acetyl-CoA carboxylase subunits from E. coli or mutants and homologues thereof; malonyl-CoA reductase 3-hydroxypropionate-forming from Chloroflexus aurantiacus or mutants and homologues thereof; malonyl-CoA reductase (malonate semialdehyde forming from Sulfolobus tokodaii str. 7 or mutants and homologues thereof, malonic semialdehyde reductase from Sulfolobus tokodaii str. 7 or mutants and homologues thereof, aldehyde dehydrogenase/alcohol dehydrogenase 3-hydroxy-propionaldehyde dehydrogenase (gamma-Glu-gamma-aminobutyraldehyde dehydrogenase, NAD(P)H-dependent) from E. coli str. K-12 substr. MG1655 or mutants and homologues thereof; and succinic aldehyde reductase from E. coli K-12 wherein the expression increases the production of 1,3-propanediol. In a certain embodiment of the tenth embodiment, the organism is Methylophilus methylotrophus.

In the eleventh embodiment of the first aspect, the product is poly-4-hydroxybutyrate and the feedstock is methanol and the modified genetic pathway is a succinate semialdehyde dehydrogenase pathway optionally including an alpha-ketoglutarate decarboxylase pathway.

The one or more genes that are stably expressed encode one or more enzymes are selected from: succinate semialdehyde dehydrogenase, alpha-ketoglutarate decarboxylase, succinic semialdehyde reductase, CoA transferase, CoA ligase, butyrate kinase, phosphotransbutyrylase, 4-hydroxybutyryl-CoA reductase and 4-hydroxybutyrylaldehyde reductase; wherein the expression increases the production of poly-4-hydroxybutyrate. In a certain embodiment of the eleventh embodiment, the organism is Methylophilus methylotrophus.

In the twelfth embodiment of the first aspect, the product is poly-3-hydroxybutyrate-co 4-hydroxybutyrate and the feedstock is methanol and the modified genetic pathway is a succinate semialdehyde dehydrogenase pathway optionally including an alpha-ketoglutarate decarboxylase pathway or a crotonase pathway. The one or more genes that are stably expressed encode one or more enzymes are selected from: acetyl-CoA acetyltransferase; acetoacetyl-CoA reductase; succinate semialdehyde dehydrogenase, alpha-ketoglutarate decarboxylase, succinic semialdehyde reductase, CoA transferase, CoA ligase, butyrate kinase, phosphotransbutyrylase, 4-hydroxybutyryl-CoA reductase; 4-hydroxybutyrylaldehyde reductase; acetyl-CoA transferase and acetoacetyl-CoA reductase; crotonase; and polyhydroxyalkanoate synthase, wherein the expression increases the production of poly-3-hydroxybutyrate-co-4-hydroxybutyrate.

In a certain embodiment of the twelfth embodiment, the organism is Methylophilus methylotrophus or Methylobacterium extorquens having one or more of the following genes deleted: phaC1, phaC2, depA and depB.

In the thirteenth embodiment of the first aspect, wherein the product is 1,4-butanediol, and the feedstock is methanol and the modified genetic pathway is a succinate semialdehyde dehydrogenase pathway optionally including an alpha-ketoglutarate decarboxylase pathway or a crotonase pathway. The one or more genes that are stably expressed encode one or more enzymes are selected from: succinate semialdehyde dehydrogenase, alpha-ketoglutarate decarboxylase, succinic semialdehyde reductase, CoA transferase, CoA ligase, butyrate kinase, phosphotransbutyrylase, 4-hydroxybutyryl-CoA reductase; 4-hydroxybutyrylaldehyde reductase; acetyl-CoA transferase, acetoacetyl-CoA reductase, crotonase, 4-hydroxybutyryl-CoA dehydratase, 4-hydroxybutyryl-CoA reductase and 4-hydroxybutyrylaldehyde reductase, wherein the expression increases the production of 1,4-butanediol. In a certain embodiment of the thirteenth embodiment, the organism is Methylophilus methylotrophus.

In the fourteenth embodiment of the first aspect, wherein the product is poly-5-hydroxyvalerate and the feedstock is methanol and the pathway is a lysine pathway. The one or more genes that are stably expressed encode one or more enzymes are selected from lysine 2-monooxygenase, 5-aminopentanamidase or mutants and homologues thereof; aminopentanoate transaminase or mutants and homologues thereof; succinate semi aldehyde reductase or mutants and homologues thereof; CoA-transferase or mutants and homologues thereof; Co-A ligase or mutants and homologues thereof; and polyhydroxyalkanoate synthase or mutants and homologues thereof; wherein the expression increases the production of poly-5-hydroxyvalerate. In a certain embodiment of the fourteenth embodiment, the organism is Methylophilus methylotrophus.

In the fifteenth embodiment of the first aspect, the product is poly-3-hydroxybutyrate-co-5-hydroxyvalerate and the feedstock is methanol and the pathway is a lysine pathway. The one or more genes that are stably expressed encode one or more enzymes are selected from acetyl-CoA acetyltransferase or mutants and homologues thereof; acetoacetyl-CoA reductase or mutants and homologues thereof; polydroxyalkanoate synthase or mutants and homologues thereof; lysine 2-monooxygenase, 5-aminopentanamidase or mutants and homologues thereof; aminopentanoate transaminase or mutants and homologues thereof; succinate semialdehyde reductase or mutants and homologues thereof; CoA-transferase or mutants and homologues thereof; Co-A ligase or mutants and homologues thereof; and polyhroxyalkanoate synthase or mutants and homologues thereof; wherein the expression increases the production of poly-3-hydroxybutyrate-co-5-hydroxyvalerate copolymer. In a certain embodiment of the fifteenth embodiment, the organism is Methylophilus methylotrophus or Methylobacterium extorquens.

In the sixteenth embodiment of the first aspect, the product is 1,5-pentanediol and the feedstock is methanol and the pathway is a lysine pathway. The one or more genes that are stably expressed encode one or more enzymes are selected from lysine 2-monooxygenase or mutants and homologues thereof; 5-aminopentanamidase or mutants and homologues thereof; 5-aminopentanoate transaminase or mutants and homologues thereof; succinate semialdehyde reductase or mutants and homologues thereof; CoA-transferase or mutants and homologues thereof; CoA ligase or mutants and homologues thereof; CoA-dependent propionaldehyde dehydrogenase or mutants and homologues thereof; and 1,3-propanediol dehydrogenase or mutants and homologues thereof; wherein the expression increases the production of 1,5-pentanediol. In a certain embodiment of the sixteenth embodiment, the organism is Methylophilus methylotrophus.

In the seventeenth embodiment of the first aspect, the product is poly-3-hydroxypropionate, the feedstock is methane and the modified genetic pathway is a malonyl-CoA reductase metabolic pathway. The one or more genes that are stably expressed encode one or more enzymes are selected from: acetyl-CoA acetyltransferase, acetyl-CoA carboxylase, malonyl-CoA reductase (3-hydroxypropionate-forming), malonyl-CoA reductase (malonate semialdehyde-forming), malonic semialdehyde reductase, CoA transferase, CoA ligase, aldehyde dehydrogenase/alcohol dehydrogenase, coA-acylating 3-hydroxypropionaldehyde dehydrogenase, and polyhydroxyalkanoate synthase, wherein the expression increases the production of poly-3-hydroxypropionate. For example, one or more genes that are stably expressed encode one or more enzyme are selected from: an acetyl-CoA carboxylase subunits from E. coli or mutants and homologues thereof; a malonyl-CoA reductase (3-hydroxypropionate-forming) from Chloroflexus aurantiacus or mutants and homologues thereof; malonyl-CoA reductase (malonate semialdehyde-forming) from Sulfolobu tokodali str. 7 or mutants and homologues thereof; malonic semialdehyde reductase from Sulfolobu tokodaii str. 7 or mutants and homologues thereof; CoA transferase from Clostridium kluyversi DSM 555, or mutants and homologues thereof; CoA ligase from Pseudomonas putida or mutants and homologues thereof; and polyhydroxyalkanoate synthase from a fusion protein of Pseudomonas putida and Ralstonia eutropha JMP134 or mutants and homologues thereof, wherein the expression increases the production of poly-3-hydroxypropionate. In a certain embodiment of the seventeenth embodiment, the organism is methylocystis hirsute having one or more of the following genes deleted: pha A, phaB, phaC1, phaC2, depA and depB.

In the eighteenth embodiment of the first aspect, the product is poly-3-hydroxypropionate, the feedstock is methane and the modified genetic pathway is a dihydroxyacetone-phosphate metabolic pathway. The one or more genes that are stably expressed encode one or more enzymes are selected from: glycerol-3-phosphate dehydrogenase (NAD+); glycerol-3-phosphate dehydrogenase (NADP+); glycerol-3-phosphatase; glycerol dehydratase; glycerol dehydratase reactivating enzyme; aldehyde dehydrogenase; alcohol dehydrogenase; CoA-acylating 3-hydroxypropionaldehyde dehydrogenase; and polyhydroxyalkanoate synthase, wherein the expression increases the production of poly-3-hydroxypropionate. For example, the one or more genes that are stably expressed encode one or more enzyme are selected from glycerol-3-phosphate dehydrogenase (NAD+) from Saccharomyces cerevisiae S288c or mutants and homologues thereof; glycerol-3-phosphate dehydrogenase (NADP+) from Rickettsia prowazekii (strain Madrid E) or mutants and homologues thereof; glycerol-3-phosphatase from Saccharomyces cerevisiae S288c or mutants and homologues thereof; glycerol dehydratase small, medium and large subunits from Klebsiella pneumonia or mutants and homologues thereof; glycerol dehydratase reactivating enzyme (Chain A and Chain B) from Klebsiella pneumonia or mutants and homologues thereof; aldehyde dehydrogenase/alcohol dehydrogenase from E. coli str. K-12 substr. MG1655; or mutants and homologues thereof; CoA-acylating 3-hydroxypropionaldehyde dehydrogenase from Salmonella enterica subsp. enterica serovar Typhimurium str. LT2 or mutants and homologues thereof; and polyhydroxyalkanoate synthase from a fusion protein of Pseudomonas putida and Ralstonia eutropha JMP134 or mutants and homologues thereof; wherein the expression increases the production of poly-3-hydroxypropionate. In a certain embodiment of the eighteenth embodiment, the organism is methylocystis hirsute having one or more of the following genes deleted: pha A, phaB, phaC1, phaC2, depA and depB.

In the nineteenth embodiment of the first aspect, wherein the product is poly-3-hydroxybutyrate-co-3-hydroxy propionate copolymer, the feedstock is methane and the modified genetic pathway is a malonyl-CoA reductase metabolic pathway. The one or more genes that are stably expressed encode one or more enzymes are selected from: from acetyl-CoA acetyltransferase; acetoacetyl-CoA reductase; acetyl-CoA carboxylase, malonyl-CoA reductase (3-hydroxypropionate-forming), malonyl-CoA reductase (malonate semialdehyde-forming), malonic semialdehyde reductase, CoA transferase, CoA ligase, and polyhydroxyalkanoate synthase, wherein the expression increases the production of is poly-3-hydroxybutyrate-co-3-hydroxy propionate copolymer. For example, the one or more genes that are stably expressed encode one or more enzyme are selected from acetyl-CoA acetyltransferase from Zoogloea ramigera or mutants and homologues thereof; acetoacetyl-CoA reductase from Zoogloea ramigera or mutants and homologues thereof; an acetyl-CoA carboxylase subunits from E. coli or mutants and homologues thereof; a malonyl-CoA reductase (3-hydroxypropionate-forming) from Chloroflexus aurantiacus or mutants and homologues thereof; malonyl-CoA reductase (malonate semialdehyde-forming) from Sulfolobus tokodaii str. 7 or mutants and homologues thereof; malonic semialdehyde reductase from Sulfolobus tokodaii str. 7 or mutants and homologues thereof; CoA transferase from Clostridium kluyveri DSM 555, or mutants and homologues thereof; CoA ligase from Pseudomonas putida or mutants and homologues thereof; and polyhydroxyalkanoate synthase from a fusion protein of Pseudomonas putida and Ralstonia eutropha JMP134 or mutants and homologues thereof; wherein the expression increases the production of poly-3-hydroxybutyrate-co-3-hydroxyproprionate copolymer. In a certain embodiment of the nineteenth embodiment, the organism is methylocystis hirsute having one or more of the following genes deleted: phaC1, phaC2, depA and depB.

In the twentieth embodiment of the first aspect, wherein the product is poly-3-hydroxybutyrate-co-3-hydroxypropionate copolymer, the feedstock is methane and the modified genetic pathway is a dihydroxyacetone-phosphate metabolic pathway. The one or more genes that are stably expressed encode one or more enzymes are selected from: from acetyl-CoA acetyltransferase; acetoacetyl-CoA reductase; glycerol-3-phosphate dehydrogenase (NAD+); glycerol-3-phosphate dehydrogenase (NADP+); glycerol-3-phosphatase; glycerol dehydratase; glycerol dehydratase reactivating enzyme; aldehyde dehydrogenase; alcohol dehydrogenase; CoA-acylating 3-hydroxypropionaldehyde dehydrogenase; and polyhydroxyalkanoate synthase, wherein the expression increases the production of poly-3-hydroxybutyrate-co-3-hydroxy-propionate copolymer. For example, the one or more genes that are stably expressed encode one or more enzyme are selected from: acetyl-CoA acetyltransferase from Zoogloea ramigera or mutants and homologues thereof; acetoacetyl-CoA reductase from Zoogloea ramigera or mutants and homologues thereof glycerol-3-phosphate dehydrogenase (NAD+) from Saccharomyces cerevisiae S288c or mutants and homologues thereof; glycerol-3-phosphate dehydrogenase (NADP+) from Rickettsia prowazekii (strain Madrid E) or mutants and homologues thereof; glycerol-3-phosphatase from Saccharomyces cerevisiae S288c or mutants and homologues thereof; glycerol dehydratase small, medium and large subunits from Klebsiella pneumonia or mutants and homologues thereof; glycerol dehydratase reactivating enzyme (Chain A and Chain B) from Klebsiella pneumonia or mutants and homologues thereof; aldehyde dehydrogenase/alcohol dehydrogenase from E. coli str. K-12 substr. MG1655; or mutants and homologues thereof; CoA-acylating 3-hydroxypropionaldehyde dehydrogenase from Salmonella enterica subsp. enterica serovar Typhimurium str. LT2 or mutants and homologues thereof; and polyhydroxyalkanoate synthase from a fusion protein of Pseudomonas putida and Ralstonia eutropha JMP134 or mutants and homologues thereof; wherein the expression increases the production poly-3-hydroxybutyrate-co-3-hydroxy propionate copolymer. In a certain embodiment of the twentieth embodiment, the organism is methylocystis hirsute having one or more of the following genes deleted: phaC1, phaC2, depA and depB.

In the twenty-first embodiment of the first aspect, wherein the product is 1,3-propanediol, the feedstock is methane and the modified genetic pathway is a malonyl-CoA reductase metabolic pathway. The one or more genes that are stably expressed encode one or more enzymes are selected from: -CoA carboxylase, malonyl-CoA reductase (3-hydroxypropionate-forming), malonyl-CoA reductase (malonate semialdehyde-forming), malonic semialdehyde reductase, CoA transferase, CoA ligase, and polyhydroxyalkanoate synthase, wherein the expression increases the production of is 1,3-propanediol. For example, the one or more genes that are stably expressed encode one or more enzyme are selected an acetyl-CoA carboxylase subunits from E. coli or mutants and homologues thereof; a malonyl-CoA reductase (3-hydroxypropionate-forming) from Chloroflexus aurantiacus or mutants and homologues thereof; malonyl-CoA reductase (malonate semialdehyde-forming) from Sulfolobus tokodaii str. 7 or mutants and homologues thereof; malonic semialdehyde reductase from Sulfolobus tokodaii str. 7 or mutants and homologues thereof; CoA transferase from Clostridium kluyveri DSM 555, or mutants and homologues thereof; CoA ligase from Pseudomonas putida or mutants and homologues thereof; and polyhydroxyalkanoate synthase from a fusion protein of Pseudomonas putida and Ralstonia eutropha JMP134 or mutants and homologues thereof; wherein the expression increases the production of 1,3-propanediol. In a certain embodiment of the twenty-first embodiment, the organism is methylocystis hirsute having one or more of the following genes deleted: pha A, phaB, phaC1, phaC2, depA and depB.

In the twenty-second embodiment of the first aspect, wherein the product is 1,3-propanediol, the feedstock is methane and the modified genetic pathway is a dihydroxyacetone-phosphate metabolic pathway. The one or more genes that are stably expressed encode one or more enzymes are selected from glycerol-3-phosphate dehydrogenase (NAD+); glycerol-3-phosphate dehydrogenase (NADP+); glycerol-3-phosphatase; glycerol dehydratase; glycerol dehydratase reactivating enzyme; aldehyde dehydrogenase; alcohol dehydrogenase; CoA-acylating 3-hydroxypropionaldehyde dehydrogenase; and polyhydroxyalkanoate synthase, wherein the expression increases the production of poly-3-hydroxypropionate. For example, the one or more genes that are stably expressed encode one or more enzyme are selected from: glycerol-3-phosphate dehydrogenase (NAD+) from Saccharomyces cerevisiae S288c or mutants and homologues thereof; glycerol-3-phosphate dehydrogenase (NADP+) from Rickettsia prowazekii (strain Madrid E) or mutants and homologues thereof; glycerol-3-phosphatase from Saccharomyces cerevisiae S288c or mutants and homologues thereof; glycerol dehydratase small, medium and large subunits from Klebsiella pneumonia or mutants and homologues thereof; glycerol dehydratase reactivating enzyme (Chain A and Chain B) from Klebsiella pneumonia or mutants and homologues thereof; aldehyde dehydrogenase/alcohol dehydrogenase from E. coli str. K-12 substr. MG1655; or mutants and homologues thereof; CoA-acylating 3-hydroxypropionaldehyde dehydrogenase from Salmonella enterica subsp, enterica serovar Typhimurium str. LT2 or mutants and homologues thereof; and polyhydroxyalkanoate synthase from a fusion protein of Pseudomonas putida and Ralstonia eutropha JMP134 or mutants and homologues thereof; wherein the expression increases the production of poly-3-hydroxypropionate. In a certain embodiment of the twenty-second embodiment, the organism is methylocystis hirsute having one or more of the following genes deleted: pha A, phaB, phaC1, phaC2, depA and depB.

In the twenty-third embodiment of the first aspect, wherein the product is poly-4-hydroxybutyrate and the feedstock is methane and the modified genetic pathway is a succinate semialdehyde dehydrogenase pathway optionally including an alpha-ketoglutarate decarboxylase pathway. The one or more genes that are stably expressed encode one or more enzymes are selected from: succinate semialdehyde dehydrogenase, alpha-ketoglutarate decarboxylase, succinic semialdehyde reductase, CoA transferase, CoA ligase, butyrate kinase, phosphotransbutyrylase, 4-hydroxybutyryl-CoA reductase and 4-hydroxybutyrylaldehyde reductase. In a certain embodiment of the twenty-third embodiment, the organism is Methylocystis hirsute having one or more of the following genes deleted: pha A, phaB, phaC1, phaC2, depA and depB.

In the twenty-fourth embodiment of the first aspect, wherein the product is poly-3-hydroxybutyrate-co-4-hydroxybutyrate and the feedstock is methane and the modified genetic pathway is a succinate semialdehyde dehydrogenase pathway optionally including an alpha-ketoglutarate decarboxylase pathway. The one or more genes that are stably expressed encode one or more enzymes are selected from: acetyl-CoA acetyltransferase; acetoacetyl-CoA reductase; succinate semialdehyde dehydrogenase, alpha-ketoglutarate decarboxylase, succinic semialdehyde reductase, CoA transferase, CoA ligase, butyrate kinase, phosphotransbutyrylase, 4-hydroxybutyryl-CoA reductase; 4-hydroxybutyrylaldehyde reductase; acetyl-CoA transferase and acetoacetyl-CoA reductase. In a certain embodiment of the twenty-fourth embodiment, the organism Methylocystis hirsute having one or more of the following genes deleted: phaC1, phaC2, depA and depB.

In the twenty-fifth embodiment of the first aspect, the product is poly-3-hydroxybutyrate-co-4-hydroxybutyrate and the feedstock is methane and the modified genetic pathway is a crotonase pathway. The one or more genes that are stably expressed encode one or more enzymes are selected from: acetyl-CoA transferase, acetoacetyl-CoA reductase, crotonase, and polylhydroxyalkanoate synthase. For example, the one or more genes that are stably expressed encode one or more enzymes are selected from: acetyl-CoA transferase, acetoacetyl-CoA reductase, crotonase, and polyhydroxyalkanoate synthase from a fusion protein of Pseudomonas putida and Ralstonia eutropha JMP134 or mutants and homologues thereof, wherein the expression increases the production of poly-3-hydroxybutyrate-co-4-hydroxybutyrate.

In a certain embodiment of the twenty-fifth embodiment, the organism Methylocystis hirsute having one or more of the following genes deleted: phaC1, phaC2, depA and depB.

In the twenty-sixth embodiment of the first aspect, the product is 1,4-butanediol, and the feedstock is methanol and the modified genetic pathway is a succinate semialdehyde dehydrogenase pathway optionally including an alpha-ketoglutarate decarboxylase pathway or a acetyl-CoA acetyltransferase pathway. The one or more genes that are stably expressed encode one or more enzymes are selected from: succinate semialdehyde dehydrogenase, alpha-ketoglutarate decarboxylase, succinic semialdehyde reductase, CoA transferase, CoA ligase, butyrate kinase, phosphotransbutyrylase, 4-hydroxybutyryl-CoA reductase; 4-hydroxybutyrylaldehyde reductase; acetyl-CoA transferase, acetoacetyl-CoA reductase, crotonase, 4-hydroxybutyryl-CoA dehydratase, 4-hydroxybutyryl-CoA reductase and 4-hydroxybutyrylaldehyde reductase. For example, the one or more genes that are stably expressed encode one or more enzymes are selected from: acetyl-CoA transferase from Zoogloea ramigera or mutants and homologues thereof, acetoacetyl-CoA reductase from Zoogloea ramigera or mutants and homologues thereof, 3-hydroxybutyryl-CoA dehydratase from Clostridium acetobutylicum ATCC 824 or mutants and homologues thereof; 4-hydroxybutyryl-CoA dehydratase from Clostridium aminobutyricum or mutants and homologues thereof; coenzyme A aceylating aldehyde dehydrogenase from Clostridium beijerinckii NCIMB 8052 4-hydroxybutyrylaldehyde and acetaldehyde dehydrogenase (aceylating) from Geobacillus thermosglucosidasium strain M10ESG or mutants and homologues thereof, wherein the expression increases the production of 1,4-butanediol. In a certain embodiment of the twenty-sixth embodiment, the organism is methylocystis hirsute having one or more of the following genes is deleted: pha A, phaB, phaC1, phaC2, depA and depB.

In the twenty-seventh embodiment of the first aspect, wherein the product is 1,4-butanediol, the feedstock is methane and the modified genetic pathway is crotonase pathway. The one or more genes that are stably expressed encode one or more enzymes are selected from: acetyl-CoA transferase, acetoacetyl-CoA reductase, crotonase, 4-hydroxybutyryl-CoA dehydratase, 4-hydroxybutyryl-CoA reductase and 4-hydroxybutyrylaldehyde reductase. In a certain embodiment of the twenty-seventh embodiment, the organism is Methylocystis hirsute having one or more of the following genes is deleted: pha A, phaB, phaC1, phaC2, depA and depB.

In the twenty-eighth embodiment of the first aspect, the product is poly-5-hydroxyvalerate and the feedstock is methane and the modified genetic pathway is a lysine pathway. The one or more genes that are stably expressed encode one or more enzymes are selected from lysine 2-monooxygenase, 5-aminopentanamidase or mutants and homologues thereof; aminopentanoate transaminase or mutants and homologues thereof; succinate semialdehyde reductase or mutants and homologues thereof; CoA-transferase or mutants and homologues thereof; Co-A ligase or mutants and homologues thereof; and polyhroxyalkanoate synthase or mutants and homologues thereof; wherein the expression increases the production of poly-5-hydroxyvalerate.

In a certain embodiment of the twenty-eighth embodiment, the organism is Methylocystis hirsute having one or more of the following genes deleted: pha A, phaB, phaC1, phaC2, depA and depB.

In the twenty-ninth embodiment of the first aspect, wherein the product is poly-3-hydroxybutyrate-co-5-hydroxyvalerate copolymer and the feedstock is methane and the pathway is an acetyl-CoA pathway. The one or more genes that are stably expressed encode one or more enzymes are selected from acetyl-CoA acetyltransferase or mutants and homologues thereof; acetoacetyl-CoA reductase or mutants and homologues thereof; and polyhydroxyalkanoate synthase or mutants and homologues thereof; wherein the expression increases the production of poly-3-hydroxy butyrate-co-5-hydroxyvalerate. For example, the one or more genes that are stably expressed encode one or more enzymes are selected from acetyl-CoA acetyltransferase from Zoogloea ramigera or mutants and homologues thereof, acetoacetyl-CoA reductase from Zoogloea ramigera or mutants and homologues thereof and polyhydroxyalkanoate synthase from a fusion protein of Pseudomonas putida and Ralstonia eutropha JMP134 or mutants and homologues thereof, wherein the expression increases production of poly-3-hydroxybutyrate-co-5-hydroxyvalerate copolymer In a certain embodiment of the twenty-ninth embodiment, the organism is methylocystis hirsute having one or more of the following genes deleted: phaC1, phaC2, depA and depB.

In the thirtieth embodiment of the first aspect, wherein the product is 1,5-pentanediol and the feedstock is methane, the modified genetic pathway is a lysine pathway. The one or more genes that are stably expressed encoding one or more enzymes are selected from lysine 2-monooxygenase or mutants and homologues thereof; 5-aminopentanamidase or mutants and homologues thereof; 5-aminopentanoate transaminase or mutants and homologues thereof; succinate semialdehyde reductase or mutants and homologues thereof; CoA-transferase or mutants and homologues thereof; CoA ligase or mutants and homologues thereof; CoA-dependent propionaldehyde dehydrogenase or mutants and homologues thereof; and 1,3-propanediol dehydrogenase or mutants and homologues thereof; wherein the expression increases the production of 1,5-pentanediol. In a certain embodiment of the thirtieth embodiment, the organism is Methylocystis hirsute having one or more of the following genes deleted: pha A, phaB, phaC1, phaC2, depA and depB.

In any of the aspects or embodiments described above, the method further includes culturing a genetically engineered organism with a renewable feedstock to produce a biomass.

A second aspect of the invention is the biomass produced by any of the aspects or embodiments described above. In a certain embodiment of the second aspect, the genetically engineered organism produces a biomass and the biomass is converted to a 3-carbon product, a 4-carbon product or a 5-carbon product. In another embodiment of the second aspect included any embodiment described, the biomass is pyrolyzed. In a particular aspect, the biomass is P3HP and the product is acrylic acid; or biomass is P4HB and the product is gamma-butyrolactone or the biomass is P5HV and the product is delta-valerolactone.

In particular embodiments of any of the aspects or embodiments described above, the methylotroph organism is selected from: Methylophilus methylotrophus AS-1; Methylocystis hirsute; Methylophilus methylotrophus M12-4, Methylophilus methylotrophus M1, Methylophilus methylotrophus sp. (deposited at NCIMB as Acc. No. 11809), Methylophilus leisingeri, Methylophilus flavus sp. nov., Methylophilus luteus sp. nov., Methylomonas sp. strain 16a, Methylomonas methanica MC09, Methylobacterium extorquens AM1 (formerly known as Pseudomonas AM1), Methylococcus capsulatus Bath, Methylomonas sp. strain J, Methylomonas aurantiaca, Methylomonas fbdinarum, Methylomonas scandinavica, Methylomonas rubra, Methylomonas streptobacterium, Methylomonas rubrum, Methylomonas rosaceous, Methylobacter chroococcum, Methylobacter bovis, Methylobacter capsulatus, Methylobacter vinelandii, Methylococcus minimus, Methylosinus sporium, Methylocystis parvus, Methylocystis hirsute, Methylobacterium organophilum, Methylobacterium rhodesianum, Methylobacterium R6, Methylobacterium aminovorans, Methylobacterium chloromethanicum, Methylobacterium dichloromethanicum, Methylobacterium fujisawaense, Methylobacterium mesophilicum, Methylobacterium radiotolerans, Methylobacterium rhodinum, Methylobacterium thiocyanatum, Methylobacterium zatmanii, Methylomonas methanica, Methylomonas albus, Methylomonas agile, Methylomonas P11, Methylobacillus glycogenes, Methylosinus trichosporium, Hyphomicrobium methylovorum, Hyphomicrobium zavarzinii, Bacillus methanolicus, Bacillus cereus M-33-1, Streptomyces 239, Mycobacterium vaccae, Diplococcus PAR, Prutaminobacter ruber, Rhodopseudomonas acidophila, Arthrobacter rufescens, Arthrobacter 1A1 and 1A2, Arthrobacter 2B2, Arthrobacter globiformis SK-200, Klebsiella 101, Pseudomonas 135, Pseudomonas oleovorans, Pseudomonas rosea (NCIB 10597 to 10612), Pseudomonas extorquens (NCIB 9399), Pseudomonas PRL-W4, Pseudomonas AM1 (NCIB 9133), Pseudomonas AM2, Pseudomonas M27, Pseudomonas PP, Pseudomonas 3A2, Pseudomonas RJ1, Pseudomonas TP1, Pseudomonas sp. 1 and 135, Pseudomonas sp. YR, JB1 and PCTN, Pseudomonas methylica sp. 2 and 15, Pseudomonas 2941, Pseudomonas AT2, Pseudomonas 80, Pseudomonas aminovorans, Pseudomonas sp. 1A3, 1B1, 7B1 and 8B1, Pseudomonas S25, Pseudomonas (methylica) 20, Pseudomonas W1, Pseudomonas W6 (MB53), Pseudomonas C, Pseudomonas MA, Pseudomonas MS. Exemplary yeast strains include: Pichia pastoris, Gliocladium deliquescens, Paecilomyces varioti, Trichoderma lignorum, Hansenula polymorpha DL-1 (ATCC 26012), Hansenula polymorpha (CBS 4732), Hansenula capsulata (CBS 1993), Hansenula lycozyma (CBS 5766), Hansenula henricii (CBS 5765), Hansenula minuta (CBS 1708), Hansenula nonfermentans (CBS 5764), Hansenula philodenda (CBS), Hansenula wickerhamii (CBS 4307), Hansenula ofuaensis, Candida boidinii (ATCC 32195), Candida boidinii (CBS 2428, 2429), Candida boidinii KM-2, Candida boidinil NRRL Y-2332, Candida boidinii S-1, Candida boidinii S-2, Candida boidinii 25-A, Candida alcamigas, Candida methanolica, Candida parapsilosis, Candida utilis (ATCC 26387), Candida sp. N-16 and N-17, Kloeckera sp. 2201, Kloeckera sp. A2, Pichia pinus (CBS 5098), Pichia pinus (CBS 744), Pichia pinus NRRL YB-4025, Pichia haplophila (CBS 2028), Pichia pastoris (CBS 704), Pichia pastoris (IFP 206), Pichia trehalophila (CBS 5361), Pichia lidnerii, Pichia methanolica, Pichia methanothermo, Pichia sp. NRRL-Y-11328, Saccharomyces H-1, Torulopsis pinus (CBS 970), Torulopsis nitatophila (CBS 2027), Torulopsis nemodendra (CBS 6280), Torulopsis molishiana, Torulopsis methanolovescens, Torulopsis glabrata, Torulopsis enoki, Torulopsis methanophiles, Torulopsis methanosorbosa, Torulopsis methanodomercquil, Torulopsis nagoyaensis, Torulopsis sp. A1, Rhodotorula sp., Rhodotorula glutinis (strain cy), and Sporobolomyces roseus (strain y).

The biomass (C3 product, polymer or copolymer; C4 product, polymer or copolymer; C5 product, polymer or copolymer) can then be treated to produce versatile intermediates that can be further processed to yield desired commodity and specialty products. For example, acrylic acid can be produced from a C3 product, polymer or copolymer; gamma-butyrolactone (GBL) can be produced from a C4 product, polymer or copolymer by heat and enzymatic treatment that may further be processed for production of other desired commodity and specialty products, for example 1,4-butanediol (BDO), tetrahydrofuran (THF), N-methylpyrrolidone (NMP), N-ethylpyrrolidone (NEP), 2-pyrrolidinone, N-vinylpyrrolidone (NVP), polyvinylpyrrolidone (PVP) and the like. Others include succinic acid, 1,4-butanediamide, succinonitrile, succinamide, and 2-pyrrolidone (2-Py); and C5 product, polymer or copolymer can produce delta-valerolactone and other C5 chemicals.

Additionally, the expended (residual) PHA reduced biomass can be further utilized for energy development, for example as a fuel to generate process steam and/or heat.

### BRIEF DESCRIPTION OF THE DRAWINGS

FIG. 1 is a schematic diagram of exemplary pathways to P3HP homopolymer, P(3HB-co-3HP) copolymer, and PDO showing reactions that were modified or introduced in the Examples or that could be modified in the future in methylotrophic bacteria. Both Ac-Co A and DHAP are central metabolites produced from either methane or methanol as sole carbon source. Abbreviations: “Ac-CoA”, acetyl-CoA; “AcAc-CoA”, acetoacetyl-CoA; “3HB-CoA”, 3-hydroxybutyryl-CoA; “Mal-CoA”, malonyl-CoA; “MSA”, malonate semialdehyde; “3HP”, 3-hydroxypropionate; “3HP-CoA”, 3-hydroxypropionyl-CoA; “DHAP”, dihydroxyacetone-phosphate; “Gol-3P”, sn-glycerol-3-phosphate; “Gol”, glycerol; “3HPA”, 3-hydroxypropionaldehyde; “P3HP”, poly(3-hydroxypropionate); P(3HB-co-3HP)”, poly(3-hydroxybutyrate-co-3-hydroxypropionate); “PDO”, 1,3-propanediol. Numbered reactions: “1”, acetyl-CoA acetyltransferase (a.k.a. beta-ketothiolase); “2”, acetoacetyl-CoA reductase; “3”, acetyl-CoA carboxylase; “4”, malonyl-CoA reductase (3-hydroxypropionate-forming); “5”, malonyl-CoA reductase (malonate semialdehyde-forming); “6”, malonic semialdehyde reductase; “7”, CoA transferase or CoA ligase; “8”, glycerol-3-phosphate dehydrogenase (NAD+) or glycerol-3-phosphate dehydrogenase (NADP+); “9”, glycerol-3-phosphatase; “10”, glycerol dehydratase and glycerol dehydratase reactivating enzymes; “11”, aldehyde dehydrogenase/alcohol dehydrogenase; “12”, CoA-acylating 3-hydroxypropionaldehyde dehydrogenase; “13”, polyhydroxyalkanoate synthase; “14”, aldehyde reductase.

FIG. 2 is a schematic diagram of exemplary pathways to P4HB homopolymer, P(3HB-co-4HB) copolymer, and BDO showing reactions that were modified or introduced in the Examples or that could be modified in the future in methylotrophic bacteria. Ac-CoA, αKG, and Suc-CoA are central metabolites produced from either methane or methanol as sole carbon source. Abbreviations: “Ac-CoA”, acetyl-CoA; “AcAc-CoA”, acetoacetyl-CoA; “3HB-CoA”, 3-hydroxybutyryl-CoA; “Suc-CoA”, succinyl-CoA; “αKG”, alpha-ketoglutarate; “SSA”, succinic semialdehyde; “4HB”, 4-hydroxybutyrate; “4HB-CoA”, 4-hydroxybutyryl-CoA; “4HB-P”, 4-hydroxybutyryl-phosphate; “Crot-CoA”, crotonyl-CoA; “4HBA”, 4-hydroxybutyrylaldehyde; “P4HB”, poly(4-hydroxybutyrate); P(3HB-co-4HB)”, poly(3-hydroxybutyrate-co-4-hydroxybutyrate); “BDO”, 1,4-butanediol. Numbered reactions: “1”, acetyl-CoA acetyltransferase (a.k.a. beta-ketothiolase); “2”, acetoacetyl-CoA reductase; “3”, succinate semialdehyde dehydrogenase; “4”, alpha-ketoglutarate decarboxylase, also known as 2-oxoglutarate decarboxylase; “5”, succinic semialdehyde reductase; “6”, CoA transferase or CoA ligase; “7”, butyrate kinase; “8”, phosphotransbutyrylase; “9”, crotonase; “10”, 4-hydroxybutyryl-CoA dehydratase; “1”, polyhydroxyalkanoate synthase; “12”, 4-hydroxybutyryl-CoA reductase; “13”, 4-hydroxybutyrylaldehyde reductase.

FIG. 3 is a schematic diagram of exemplary pathways to P5HV homopolymer, P(3HB-co-5HV) copolymer, and 1,5PD showing reactions that were modified or introduced in the Examples or that could be modified in the future in methylotrophic bacteria. Both Ac-CoA and Lys are central metabolites produced from either methane or methanol as sole carbon source. Abbreviations: “Ac-CoA”, acetyl-CoA; “AcAc-CoA”, acetoacetyl-CoA; “3HB-CoA”, 3-hydroxybutyryl-CoA; “Lys”, L-lysine; “5APA”, 5-aminopentanamide; “5APO”, 5-aminopentanoate; “GSA”, glutarate semialdehyde; “5HV”, 5-hydroxyvalerate; “5HV-CoA”, 5-hydroxyvaleryl-CoA; “5HVA”, 5-hydroxyvalerylaldehyde; “P5HV”, poly(5-hydroxyvalerate); P(3HB-co-5HV)”, poly(3-hydroxybutyrate-co-5-hydroxyvalerate); “1,5PD”, 1,5-pentanediol. Numbered reactions: “1”, acetyl-CoA acetyltransferase (a.k.a. beta-ketothiolase); “2”, acetoacetyl-CoA reductase; “3”, lysine 2-monooxygenase; “4”, 5-aminopentanamidase; “5”, 5-aminopentanoate transaminase; “6”, succinate semialdehyde reductase; “7”, CoA-transferase or CoA ligase; “8”, CoA-dependent propionaldehyde dehydrogenase; “9”, 1,3-propanediol dehydrogenase; “10”, polyhydroxyalkanoate synthase.

FIG. 4 GC-MS chromatogram of compounds obtained from pyrolysis (@25° C.) of Methylophilus methylotrophus AS-1 biomass+P3HP produced using methanol feedstock. Peak at 4.05-4.12 minutes is shown to be acrylic acid or 2-propenoic acid as shown by the mass spectral library match.

### DETAILED DESCRIPTION OF THE INVENTION

Methods of increasing the production of a 3-carbon (C3) product or polymer of 3-carbon monomers, 4-carbon (C4) product or a polymer of 4-carbon monomers, or 5-carbon (C5) product or polymer of 5-carbon monomers or copolymers thereof from methanol or methane in methylotrophic bacteria are described herein. Metabolic pathways are genetically engineered in microorganisms by providing one or more genes that are stably expressed that encodes an enzyme with an activity catalyzing the methanol or methane to produce the carbon products, polymer or copolymers, wherein growth is improved and the carbon flux from the renewable feedstock is increased.

In the 3-carbon, 4-carbon and 5-pathways described herein, one or more enzymes, mutants or homologues thereof may be included or modified in the methylotrophic bacteria to produce a desired 3-carbon product, 4-carbon product or 5-carbon product, or polymers or copolymers thereof. These pathways provide increased yield of desired products that can be cultured using methanol or methane as a feedstock and produced in quantities that are a viable, cost effective alternative to petroleum based products.

In the 3-carbon pathways, both acetyl CoA and dihydroxyacetone phosphate are central metabolites produced from either methane or methanol as sole carbon source. The enzymes in the 3-carbon pathways include acetyl-CoA acetyltransferase (a.k.a. beta-ketothiolase); acetoacetyl-CoA reductase; acetyl-CoA carboxylase; malonyl-CoA reductase (3-hydroxypropionate-forming); malonyl-CoA reductase (malonate semialdehyde-forming); malonic semialdehyde reductase; CoA transferase or CoA ligase; glycerol-3-phosphate dehydrogenase (NAD+) or glycerol-3-phosphate dehydrogenase (NADP+); glycerol-3-phosphatase; glycerol dehydratase and glycerol dehydratase reactivating enzymes; aldehyde dehydrogenase/alcohol dehydrogenase; CoA-acylating 3-hydroxypropionaldehyde dehydrogenase; polyhydroxyalkanoate synthase and aldehyde reductase.

For exemplary pathways for P4HB homopolymer, P(3HB-co-4HB) copolymer, and BDO, one or more enzymes or mutants or homologues thereof may be introduced including pathways for Ac-CoA, αKG, and Suc-CoA produced from either methane or methanol as sole carbon source. The enzymes include acetyl-CoA acetyltransferase (a.k.a. beta-ketothiolase); acetoacetyl-CoA reductase; succinate semialdehyde dehydrogenase; alpha-ketoglutarate decarboxylase, also known as 2-oxoglutarate decarboxylase; succinic semialdehyde reductase; CoA transferase or CoA ligase; butyrate kinase; phosphotransbutyrylase; crotonase; 4-hydroxybutyryl-CoA dehydratase; polyhydroxyalkanoate synthase; 4-hydroxybutyryl-CoA reductase; 4-hydroxybutyrylaldehyde reductase.

Exemplary pathways to produce P5HV homopolymer, P(3HB-co-5HV) copolymer, and 1,5-pentanediol (1,5PD) with reactions that can be modified or introduced include Ac-CoA and Lysine pathways. The enzymes include acetyl-CoA acetyltransferase (a.k.a. beta-ketothiolase); acetoacetyl-CoA reductase; lysine 2-monooxygenase; 5-aminopentanamidase; 5-aminopentanoate transaminase; succinate semialdehyde reductase; CoA-transferase or CoA ligase; CoA-dependent propionaldehyde dehydrogenase; 1,3-propanediol dehydrogenase; and polyhydroxyalkanoate synthase.

The level of P3HB or P3HP, 3-carbon (C3) product, or polymer of 3-carbon monomers, P4HB, 4-carbon (C4) product or a polymer of 4-carbon monomers, or 5-carbon (C5) product, or polymer of 5-carbon monomers, or copolymers of these monomers produced in the biomass from the renewable substrate is greater than 5% (e.g., about 10%, about 20%, about 30%, about 40%, about 50%, about 60%, about 70%, about 80%) of the total dry weight of the biomass. The biomass is then available for post purification and modification methodologies to produce other biobased chemicals and derivatives.

Producing C3, C4 and C5 Chemicals from the Biomass

In general, during or following production (e.g., culturing) of the PHA polymer or carbon chemical product biomass, the biomass is optionally combined with a catalyst under suitable conditions to help convert the PHA polymer or chemical product to a C3, C4 or C5 product (e.g., acrylic acid, gamma-butyrolactone, or delta-valerolactone). The catalyst (in solid or solution form) and biomass are combined for example by mixing, flocculation, centrifuging or spray drying, or other suitable method known in the art for promoting the interaction of the biomass and catalyst driving an efficient and specific conversion of polymer to product (e.g., P4HB to gamma-butyrolactone). In some embodiments, the biomass is initially dried, for example at a temperature between about 100° C. and about 150° C. and for an amount of time to reduce the water content of the biomass. The dried biomass is then re-suspended in water prior to combining with the catalyst. Suitable temperatures and duration for drying are determined for product purity and yield and can in some embodiments include low temperatures for removing water (such as between 25° C. and 150° C.) for an extended period of time or in other embodiments can include drying at a high temperature (e.g., above 450° C.) for a short duration of time. Under “suitable conditions” refers to conditions that promote the catalytic reaction. For example, under conditions that maximize the generation of the product such as in the presence of co-agents or other material that contributes to the reaction efficiency. Other suitable conditions include in the absence of impurities, such as metals or other materials that would hinder the reaction from progressing.

As used herein, “catalyst” refers to a substance that initiates or accelerates a chemical reaction without itself being affected or consumed in the reaction. Examples of useful catalysts include metal catalysts. In certain embodiments, the catalyst lowers the temperature for initiation of thermal decomposition and increases the rate of thermal decomposition at certain pyrolysis temperatures (e.g., about 200° C. to about 325° C.).

In some embodiments, the catalyst is a chloride, oxide, hydroxide, nitrate, phosphate, sulphonate, carbonate or stearate compound containing a metal ion. Examples of suitable metal ions include aluminum, antimony, barium, bismuth, cadmium, calcium, cerium, chromium, cobalt, copper, gallium, iron, lanthanum, lead, lithium, magnesium, molybdenum, nickel, palladium, potassium, silver, sodium, strontium, tin, tungsten, vanadium or zinc and the like. In some embodiments, the catalyst is an organic catalyst that is an amine, azide, enol, glycol, quaternary ammonium salt, phenoxide, cyanate, thiocyanate, dialkyl amide and alkyl thiolate. In some embodiments, the catalyst is calcium hydroxide. In other embodiments, the catalyst is sodium carbonate. Mixtures of two or more catalysts are also included.

In certain embodiments, the amount of metal catalyst is about 0.1% to about 15% or about 1% to about 25%, or about 4% to about 50% based on the weight of metal ion relative to the dry solid weight of the biomass. In some embodiments, the amount of catalyst is between about 7.5% and about 12%. In other embodiments, the amount of catalyst is about 0.5% dry cell weight, about 1%, about 2%, about 3%, about 4%, about 5, about 6%, about 7%, about 8%, about 9%, or about 10%, or about 11%, or about 12%, or about 13%, or about 14%, or about 15%, or about 20%, or about 30%, or about 40% or about 50% or amounts in between these.

As used herein, the term “sufficient amount” when used in reference to a chemical reagent in a reaction is intended to mean a quantity of the reference reagent that can meet the demands of the chemical reaction and the desired purity of the product.

### Thermal Degradation of the Biomass to Carbon Products

In certain embodiments, the biomass titer (g/L) of carbon product has been increased when compared to the host without the overexpression or inhibition of one or more genes in the carbon pathway. In certain embodiments, the product titer is reported as a percent dry cell weight (% dcw) or as grams of product/Kg biomass.

“Heating,” “pyrolysis”, “thermolysis” and “torrefying” as used herein refer to thermal degradation (e.g., decomposition) of the P4HB biomass for conversion to C4 products. In general, the thermal degradation of the P4HB biomass occurs at an elevated temperature in the presence of a catalyst. For example, in certain embodiments, the heating temperature for the processes described herein is between about 200° C. to about 400° C. In some embodiments, the heating temperature is about 200° C. to about 350° C. In other embodiments, the heating temperature is about 300° C. “Pyrolysis” typically refers to a thermochemical decomposition of the biomass at elevated temperatures over a period of time. The duration can range from a few seconds to hours. In certain conditions, pyrolysis occurs in the absence of oxygen or in the presence of a limited amount of oxygen to avoid oxygenation. The processes for P4HB biomass pyrolysis can include direct heat transfer or indirect heat transfer. “Flash pyrolysis” refers to quickly heating the biomass at a high temperature for fast decomposition of the P4HB biomass, for example, depolymerization of a P4HB in the biomass. Another example of flash pyrolysis is RTP™ rapid thermal pyrolysis. RTP™ technology and equipment from Envergent Technologies, Des Plaines, Ill. converts feedstocks into bio-oil. “Torrefying” refers to the process of torrefaction, which is an art-recognized term that refers to the drying of biomass. The process typically involves heating a biomass in a temperature range from 200-350° C., over a relatively long duration (e.g., 10-30 minutes), typically in the absence of oxygen. The process results for example, in a torrefied biomass having a water content that is less than 7 wt % of the biomass. The torrefied biomass may then be processed further. In some embodiments, the heating is done in a vacuum, at atmospheric pressure or under controlled pressure. In certain embodiments, the heating is accomplished without the use or with a reduced use of petroleum generated energy.

In certain embodiments, the biomass is dried prior to heating. Alternatively, in other embodiments, drying is done during the thermal degradation (e.g., heating, pyrolysis or torrefaction) of the biomass. Drying reduces the water content of the biomass. In certain embodiments, the biomass is dried at a temperature of between about 100° C. to about 350° C., for example, between about 200° C. and about 275° C. In some embodiments, the dried biomass has a water content of 5 wt %, or less.

In certain embodiments, the heating of the biomass/catalyst mixture is carried out for a sufficient time to efficiently and specifically convert the biomass to a carbon product. In certain embodiments, the time period for heating is from about 30 seconds to about 1 minute, from about 30 seconds to about 1.5 minutes, from about 1 minute to about 10 minutes, from about 1 minute to about 5 minutes or a time between, for example, about 1 minute, about 2 minutes, about 1.5 minutes, about 2.5 minutes, about 3.5 minutes.

In other embodiments, the time period is from about 1 minute to about 2 minutes. In still other embodiments, the heating time duration is for a time between about 5 minutes and about 30 minutes, between about 30 minutes and about 2 hours, or between about 2 hours and about 10 hours or for greater that 10 hours (e.g., 24 hours).

In certain embodiments, the heating temperature is at a temperature of about 200° C. to about 350° C. including a temperature between, for example, about 205° C., about 210° C., about 215° C., about 220° C., about 225° C., about 230° C., about 235° C., about 240° C., about 245° C., about 250° C., about 255° C. about 260° C., about 270° C., about 275° C., about 280° C., about 290° C., about 300° C., about 310° C., about 320° C., about 330° C., about 340° C., or 345° C. In certain embodiments, the temperature is about 250° C. In certain embodiments, the temperature is about 275° C. In other embodiments, the temperature is about 300° C.

In certain embodiments, the process also includes flash pyrolyzing the residual biomass for example at a temperature of 500° C. or greater for a time period sufficient to decompose at least a portion of the residual biomass into pyrolysis liquids. In certain embodiments, the flash pyrolyzing is conducted at a temperature of 500° C. to 750° C. In some embodiments, a residence time of the residual biomass in the flash pyrolyzing is from 1 second to 15 seconds, or from 1 second to 5 seconds or for a sufficient time to pyrolyze the biomass to generate the desired pyrolysis precuts, for example, pyrolysis liquids. In some embodiments, the flash pyrolysis can take place instead of torrefaction. In other embodiments, the flash pyrolysis can take place after the torrefication process is complete.

As used herein, “pyrolysis liquids” are defined as a low viscosity fluid with up to 15-20% water, typically containing sugars, aldehydes, furans, ketones, alcohols, carboxylic acids and lignins. Also known as bio-oil, this material is produced by pyrolysis, typically fast pyrolysis of biomass at a temperature that is sufficient to decompose at least a portion of the biomass into recoverable gases and liquids that may solidify on standing. In some embodiments, the temperature that is sufficient to decompose the biomass is a temperature between 400° C. to 800° C.

In certain embodiments, “recovering” the carbon product vapor includes condensing the vapor. As used herein, the term “recovering” as it applies to the vapor means to isolate it from the P4HB biomass materials, for example including but not limited to: recovering by condensation, separation methodologies, such as the use of membranes, gas (e.g., vapor) phase separation, such as distillation, and the like. Thus, the recovering may be accomplished via a condensation mechanism that captures the monomer component vapor, condenses the monomer component vapor to a liquid form and transfers it away from the biomass materials.

As a non-limiting example, the condensing of the vapor may be described as follows. The incoming gas/vapor stream from the pyrolysis/torrefaction chamber enters an interchanger, where the gas/vapor stream may be pre-cooled. The gas/vapor stream then passes through a chiller where the temperature of the gas/vapor stream is lowered to that required to condense the designated vapors from the gas by indirect contact with a refrigerant. The gas and condensed vapors flow from the chiller into a separator, where the condensed vapors are collected in the bottom. The gas, free of the vapors, flows from the separator, passes through the Interchanger and exits the unit. The recovered liquids flow, or are pumped, from the bottom of the separator to storage. For some of the products, the condensed vapors solidify and the solid is collected.

In certain embodiments, recovery of the catalyst is further included in the processes of the invention. For example, when a calcium catalyst is used calcination is a useful recovery technique. Calcination is a thermal treatment process that is carried out on minerals, metals or ores to change the materials through decarboxylation, dehydration, devolatilization of organic matter, phase transformation or oxidation. The process is normally carried out in reactors such as hearth furnaces, shaft furnaces, rotary kilns or more recently fluidized beds reactors. The calcination temperature is chosen to be below the melting point of the substrate but above its decomposition or phase transition temperature. Often this is taken as the temperature at which the Gibbs free energy of reaction is equal to zero. For the decomposition of CaCO₃ to CaO, the calcination temperature at ΔG=0 is calculated to be ˜850° C. Typically for most minerals, the calcination temperature is in the range of 800-1000° C.

To recover the calcium catalyst from the biomass after recovery of the C4 product, one would transfer the spent biomass residue directly from pyrolysis or torrefaction into a calcining reactor and continue heating the biomass residue in air to 825-850° C. for a period of time to remove all traces of the organic biomass. Once the organic biomass is removed, the catalyst could be used as is or purified further by separating the metal oxides present (from the fermentation media and catalyst) based on density using equipment known to those in the art.

In other embodiments, the product can be further purified if needed by additional methods known in the art, for example, by distillation, by reactive distillation by treatment with activated carbon for removal of color and/or odor bodies, by ion exchange treatment, by liquid-liquid extraction—with an immiscible solvent to remove fatty acids etc, for purification after recovery, by vacuum distillation, by extraction distillation or using similar methods that would result in further purifying product to increase the yield of product. Combinations of these treatments can also be utilized.

As used herein, the term “residual biomass” refers to the biomass after PHA conversion to the small molecule intermediates. The residual biomass may then be converted via torrefaction to a useable, fuel, thereby reducing the waste from PHA production and gaining additional valuable commodity chemicals from typical torrefaction processes. The torrefaction is conducted at a temperature that is sufficient to densify the residual biomass. In certain embodiments, processes described herein are integrated with a torrefaction process where the residual biomass continues to be thermally treated once the volatile chemical intermediates have been released to provide a fuel material. Fuel materials produced by this process are used for direct combustion or further treated to produce pyrolysis liquids or syngas. Overall, the process has the added advantage that the residual biomass is converted to a higher value fuel which can then be used for the production of electricity and steam to provide energy for the process thereby eliminating the need for waste treatment.

A “carbon footprint” is a measure of the impact the processes have on the environment, and in particular climate change. It relates to the amount of greenhouse gases produced.

In certain embodiments, it may be desirable to label the constituents of the biomass. For example, it may be useful to deliberately label with an isotope of carbon (e.g., ¹³C) to facilitate structure determination or for other means. This is achieved by growing microorganisms genetically engineered to express the constituents, e.g., polymers, but instead of the usual media, the bacteria are grown on a growth medium with ¹³C-containing carbon source, such as glucose, pyruvic acid, etc. In this way polymers can be produced that are labeled with ¹³C uniformly, partially, or at specific sites. Additionally, labeling allows the exact percentage in bioplastics that came from renewable sources (e.g., plant derivatives) can be known via ASTM D6866—an industrial application of radiocarbon dating. ASTM D6866 measures the Carbon 14 content of biobased materials; and since fossil-based materials no longer have Carbon 14, ASTM D6866 can effectively dispel inaccurate claims of biobased content

### EXAMPLES

The present technology is further illustrated by the following examples, which should not be construed as limiting in any way.

These examples describe a number of biotechnology tools and methods for the construction of strains that generate a product of interest. Suitable host strains, the potential source and a list of recombinant genes used in these examples, suitable extrachromosomal vectors, suitable strategies and regulatory elements to control recombinant gene expression, and a selection of construction techniques to overexpress genes in or inactivate genes from host organisms are described. These biotechnology tools and methods are well known to those skilled in the art.

### Suitable Host Strains

In certain embodiments, the host strain is Methylophilus methylotrophus AS-1 (formerly known as Pseudomonas methylotropha AS-1, deposited at the National Collections of Industrial, Marine and Food Bacteria (NCIMB) as Acc. No. 10515; MacLennan et al., UK Patent No. 1370892), or Methylocystis hirsute (deposited at the Deutsche Sammlung von Mikroorganismen und Zellkulturen GmbH (DSMZ) as Acc. No. 18500; Linder et al., J. Syst. Evol. Microbiol. 57:1891-1900 (2007); Rahnama et al., Biochem. Engineer. J. 65:51-56 (2012)).

Other exemplary microbial host strains that grow on methane and/or methanol as sole carbon source include but are not limited to: Methylophilus methylotrophus M12-4, Methylophilus methylotrophus M1, Methylophilus methylotrophus sp. (deposited at NCIMB as Ace. No. 11809), Methylophilus leisingeri, Methylophilus flavus sp. nov., Methylophilus luteus sp. nov., Methylomonas sp. strain 16a, Methylomonas methanica MC09, Methylobacterium extorquens AM1 (formerly known as Pseudomonas AM1), Methylococcus capsulatus Bath, Methylomonas sp. strain J, Methylomonas aurantiaca, Methylomonas fodinarum, Methylomonas scandinavica, Methylomonas rubra, Methylomonas streptobacterium, Methylomonas rubrum, Methylomonas rosaceous, Methylobacter chroococcum, Methylobacter bovis, Methylobacter capsulatus, Methylohacter vinelandii, Methylococcus minimus, Methylosinus sporium, Methylocystis parvus, Methylocystis hirsute, Methylobacterium organophilum, Methylobacterium rhodesianum, Methylobacterium R6, Methylobacterium aminovorans, Methylobacterium chloromethanicum, Methylobacterium dichloromethanicum, Methylobacterium fujisawaense, Methylobacterium mesophilicum, Methylobacterium radiotolerans, Methylobacterium rhodinum, Methylobacterium thiocyanatum, Methylobacterium zatmanii, Methylomonas methanica, Methylomonas albus, Methylomonas agile, Methylomonas P11, Methylobacillus glycogenes, Methylosinus trichosporium, Hyphomicrobium methylovorum, Hyphomicrobium zavarzinii, Bacillus methanolicus, Bacillus cereus M-33-1, Streptomyces 239, 1Mycobacterium vaccae, Diplococcus PAR, Protaminobacter ruber, Rhodopseudomonas acidophila, Arthrobacter rufescens, Arthrobacter 1A1 and 1A2, Arthrobacter 2B2, Arthrobacter globiformis SK-200, Klebsiella 101, Pseudomonas 135, Pseudomonas oleovorans, Pseudomonas rosea (NCIB 10597 to 10612), Pseudomonas extorquens (NCIB 9399), Pseudomonas PRL-W4, Pseudomonas AM1 (NCIB 9133), Pseudomonas AM2, Pseudomonas M27, Pseudomonas PP, Pseudomonas 3A2, Pseudomonas RJ1, Pseudomonas TP1, Pseudomonas sp. 1 and 135, Pseudomonas sp. YR, JB1 and PCTN, Pseudomonas methylica sp. 2 and 15, Pseudomonas 2941, Pseudomonas AT2, Pseudomonas 80, Pseudomonas aminovorans, Pseudomonas sp. 1A3, 1B1, 7B1 and 8B1, Pseudomonas $25, Pseudomonas (methylica) 20, Pseudomonas W1, Pseudomonas W6 (MB53), Pseudomonas C, Pseudomonas MA, Pseudomonas MS. Exemplary yeast strains include: Pichia pastoris, Gliocladium deliquescens, Paecilomyces varioli, Trichoderma lignorum, Hansenula polymorpha DL-1 (ATCC 26012), Hansenula polymorpha (CBS 4732), Hansenula capsulata (CBS 1993), Hansenula lycozyma (CBS 5766), Hansenula henricii (CBS 5765), Hansenula minuta (CBS 1708), Hansenula nonfermentans (CBS 5764), Hansenula philodenda (CBS), Hansenula wickerhamii (CBS 4307), Hansenula ofuaensis, Candida boidinii (ATCC 32195), Candida boidinii (CBS 2428, 2429), Candida boidinii KM-2, Candida boidinii NRRL Y-2332, Candida boidinii S-1, Candida boidinii S-2, Candida boidinii 25-A, Candida alcamigas, Candida methanolica, Candida parapsilosis, Candida utilis (ATCC 26387), Candida sp. N-16 and N-17, Kloeckera sp. 2201, Kloeckera sp. A2, Pichia pinus (CBS 5098), Pichia pinus (CBS 744), Pichia pinus NRRL YB-4025, Pichia haplophila (CBS 2028), Pichia pastoris (CBS 704), Pichia pastoris (IFP 206), Pichia trehalophila (CBS 5361), Pichia lidnerii, Pichia methanolica, Pichia methanothermo, Pichia sp. NRRL-Y-11328, Saccharomyces H-1, Torulopsis pinus (CBS 970), Torulopsis nitatophila (CBS 2027), Torulopsis nemodendra (CBS 6280), Torulopsis molishiana, Torulopsis methanolovescens, Torulopsis glabrata, Torulopsis enoki, Torulopsis methanophiles, Torulopsis methanosorbosa, Torulopsis methanodomercquii, Torulopsis nagoyaensis, Torulopsis sp. A1, Rhodotorula sp., Rhodotorula glutinis (strain cy), and Sporobolomyces roseus (strain y).

### Source of Recombinant Genes

Sources of encoding nucleic acids for PHA biopolymers or C3, C4, and C5 biochemicals pathway enzymes can include, for example, any species where the encoded gene product is capable of catalyzing the referenced reaction. Such species include both prokaryotic and eukaryotic organisms including, but not limited to, bacteria, including archaea and eubacteria, and eukaryotes, including yeast, plant, insect, animal, and mammal, including human. Exemplary species for such sources include, for example, Escherichia coli, Saccharomyces cerevisiae, Saccharomyces kluyveri, Synechocystis sp. PCC 6803, Synechococcus elongatus PCC 7942, Synechococcus sp. PCC 7002, Chlorogleopsis sp. PCC 6912, Chloroflexus aurantiacus, Clostridium kluyveri, Clostridium acetobutylicum, Clostridium beijerinckii, Clostridium saccharoperbutylacetonicum, Clostridium perjringens, Clostridium difficile, Clostridium botulinum, Clostridium tyrobutyricum, Clostridium tetanomorphum, Clostridium tetani, Clostridium propionicum, Clostridium aminobutyricum, Clostridium subterminale, Clostridium sticklandii, Ralstonia eutropha, Mycobacterium bovis, Mycobacterium tuberculosis, Porphyromonas gingivalis, Arabidopsis thaliana, Thermus thermophilus, Pseudomonas species, including Pseudomonas aeruginosa, Pseudomonas putida, Pseudomonas stutzeri, Pseudomonas fluorescens, Chlorella minutissima, Chlorella emersonii, Chlorella sorokiniana, Chlorella ellipsoidea, Chlorella sp., Chlorella protothecoides, Homo sapiens, Oryctolagus cuniculus, Rhodobacter sphaeroides, Thermoanaerobacter brockii, Metallosphaera sedula, Leuconostoc mesenteroides, Roseiflexus castenholzii, Erythrobacter, Simmondsia chinensis, Acinetobacter species, including Acinetobacter calcoaceticus and Acinetobacter baylyi, Sulfolobus tokodaii, Sulfolobus solfataricus, Sulfolobus acidocaldarius, Bacillus subtilis, Bacillus cereus, Bacillus megaterium, Bacillus brevis, Bacillus pumilus, Rattus norvegicus, Klebsiella pneumonia, Klebsiella oxytoca, Euglena gracilis, Treponema denticola, Moorella thermoacetica, Thermotoga maritima, Halobacterium salinarum, Geobacillus stearothermophilus, Aeropyrum pernix, Sus scrofa, Caenorhabditis elegans, Corynebacterium glutamicum, Acidaminococcus fermentans, Lactococcus lactis, Lactobacillus plantarum, Streptococcus thermophilus, Enterobacter acrogenes, Candida, Aspergillus terreus, Pedicoccus pentosaceus, Zymomonas mobilis, Acetobacter pasteurians, Kluyveromyces lactis, Eubacterium barkeri, Bacteroides capillosus, Anaerotruncus colihominis, Natranaerobius thermophilus, Campylobacter jejuni, Haemophilus influenzae, Serratia marcescens, Citrobacter amalonaticus, Myxococcus xanthus, Fusobacterium nuleatum, Penicillium chrysogenum, marine gamma proteobacterium, butyrate-producing bacterium, and Trypanosoma brucei. Other suitable sources for recombinant genes constitute the methylotrophic organisms listed above. For example, microbial hosts (e.g., organisms) having PHA biopolymers or C3, C4, and C5 biochemicals biosynthetic production are exemplified herein with reference to a methylotrophic host. However, with the complete genome sequence available now for more than 2500 species However, with the complete genome sequence available now for more than 2,500 species (sec the world wide web at ncbi.nlm.nih.gov/genome/browse/), including microorganism genomes and a variety of yeast, fungi, plant, and mammalian genomes, the identification of genes encoding the requisite PHA biopolymers or C3, C4, and C5 biochemicals biosynthetic activity for one or more genes in related or distant species, including for example, homologues, orthologs, paralogs and nonorthologous gene displacements of known genes, and the interchange of genetic alterations between organisms is routine and well known in the art. Accordingly, the metabolic alterations enabling biosynthesis of PHA biopolymers or C3, C4, and C5 biochemicals of the invention described herein with reference to particular organisms such as Methylophilus methylotrophus and Methylocystis hirsute can be readily applied to other microorganisms, including prokaryotic and eukaryotic organisms alike. Given the teachings and guidance provided herein, those skilled in the art will know that a metabolic alteration exemplified in one organism can be applied equally to other organisms.

### Production of Transgenic Host for Producing PHA Biopolymers or C3, C4, and C5 Biochemicals

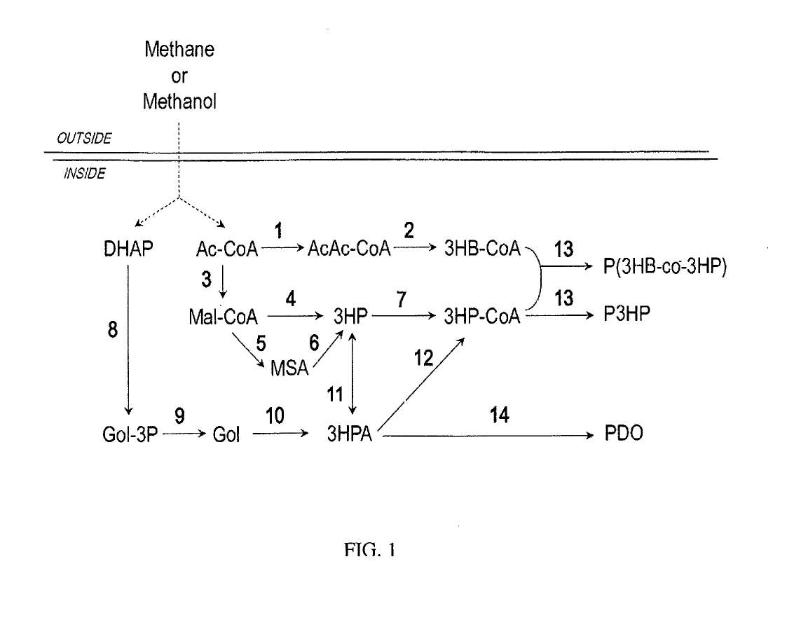

Transgenic (recombinant) hosts for producing PHA biopolymers or C3, C4, and C5 biochemicals are genetically engineered using conventional techniques known in the art. The genes cloned and/or assessed for host strains producing 3HP containing homo- and copolymers and 3-carbon biochemicals are presented below in Table 1A, along with the appropriate Enzyme Commission number (EC number) and references. Some genes were synthesized for codon optimization while others were cloned via PCR from the genomic DNA of the native or wild-type host. As used herein, “heterologous” means from another host. The host can be the same or different species. FIG. 1 shows exemplary pathways for producing P3HP, P(3HB-co-3HP), and PDO.

TABLE 1A
Genes overexpressed or deleted in microbial host strains producing 3HP-
containing PHA and 3-carbon chemicals. A star (*) after the gene name
denotes that the nucleotide sequence was optimized for expression in E. coli.

| Reaction | | | | |
|---|---|---|---|---|
| number | | | EC | |
| (FIG. 1) | Gene Name | Enzyme Name | Number | Accession No. |
| 1 | phaA5 | Acetyl-CoA | 2.3.1.9 | 2VU2_A |
| | | acetyltransferase (a.k.a. | | |
| | | beta-ketothiolase) | | |
| 2 | phaB5 | Acetoacetyl-CoA | 1.1.1.36 | P23238 |
| | | reductase | | |
| 3 | accA | Acetyl-CoA carboxylase, | 6.4.1.2 | AAC73296 |
| | | alpha subunit | | |
| 3 | accB | Acetyl-CoA carboxylase, | 6.4.1.2 | AAC76287 |
| | | BCCP (biotin carboxyl | | |
| | | carrier protein) subunit | | |
| 3 | accC | Acetyl-CoA carboxylase, | 6.4.1.2 | AAC76288 |
| | | biotin carboxylase subunit | | |
| 3 | accD | Acetyl-CoA carboxylase, | 6.4.1.2 | AAC75376 |
| | | beta (carboxyltransferase) | | |
| | | subunit | | |
| 4 | mcr_{Ca}* | Malonyl-CoA reductase | | Gene/Protein ID 1; |
| | | (3-hydroxypropionate- | | AAS20429 |
| | | forming) | | |
| 5 | mcr_{St} | Malonyl-CoA reductase | 1.2.1.75 | BAB67276 |
| | | (malonate semialdehyde- | | |
| | | forming) | | |
| 6 | msaR | Malonic semialdehyde | 1.1.1.298 | BAK54608 |
| | | reductase | | |
| 7 | orfZ | CoA transferase | 2.8.3.— | AAA92344 |
| 7 | alkK | CoA ligase (a.k.a. acyl- | 6.2.1.— | CAB54055 |
| | | CoA synthetase) | | |
| 8 | DAR1 | Glycerol-3-phosphate | 1.1.1.8 | NP_010262 |
| | (GPD1) | dehydrogenase (NAD+) | | |
| 8 | gpsA | Glycerol-3-phosphate | 1.1.1.94 | NP_220823 |
| | | dehydrogenase (NADP+) | | |
| 9 | GPP2 | glycerol-1-phosphatase | 3.1.3.21 | NP_010984 |
| | (HOR2) | HOR2 | | |
| 10 | dhaB1 | Glycerol dehydratase | 4.2.1.30 | AAA74258 |
| | | large subunit | | |
| 10 | dhaB2 | Glycerol dehydratase | 4.2.1.30 | AAA74257 |
| | | medium subunit | | |
| 10 | dhaB3 | Glycerol dehydratase | 4.2.1.30 | AAA74256 |
| | | small subunit | | |
| 10 | gdrA | Chain A, Glycerol | | (1NBW_A) AAA74255 |
| | | Dehydratase Reactivase | | |
| 10 | gdrB | Chain B, Glycerol | | 1NBW_B |
| | | Dehydratase Reactivase | | |
| 11 | puuC | 3-Hydroxy- | 1.2.1.3 | NP_415816 |
| | | propionaldehyde | | |
| | | dehydrogenase | | |
| | | (gamma-Glu-gamma- | | |
| | | aminobutyraldehyde | | |
| | | dehydrogenase, | | |
| | | NAD(P)H-dependent) | | |
| 12 | pduP | CoA-acylating 3- | 1.2.1.— | NP_460996 |
| | | hydroxypropionaldehyde | | |
| | | dehydrogenase | | |
| 13 | phaC3/C1* | Polyhydroxyalkanoate | 2.3.1.n | U.S. Patent Appl. No. |
| | | synthase fusion protein | | 2011/024612 |
| 14 | yqhD | succinic semialdehyde | 1.1.1.61 | NP_417484 |
| | | reductase | | |

Other proteins capable of catalyzing the reactions listed in Table 1A can be discovered by consulting the scientific literature, patents, BRENDA searches (http://www.brenda-enzymes.info/), and/or by BLAST searches against e.g., nucleotide or protein databases at NCBI (www.ncbi.nlm.nih.gov/). Synthetic genes can then be created to provide an easy path from sequence databases to physical DNA. Such synthetic genes are designed and fabricated from the ground up, using codons to enhance heterologous protein expression, and optimizing characteristics needed for the expression system and host. Companies such as e.g., DNA 2.0 (Menlo Park, Calif. 94025, USA) will provide such routine service. Proteins that may catalyze some of the biochemical reactions listed in Table 1A are provided in Tables 1B to 1X.

TABLE 1B
Suitable homologues for the PhaA5 protein (beta-ketothiolase,
from Zoogloea ramigera, EC No. 2.3.1.9, which acts on
acetyl-CoA + acetyl-CoA to produce acetoacetyl-CoA;
protein acc. no. 2VU2_A).

| | Protein |
|---|---|
| Protein Name | Accession No. |
| acetyl-CoA acetyltransferase | YP_002827756 |
| acetyl-CoA acetyltransferase | YP_002283310 |
| acetyl-CoA acetyltransferase | YP_002733453 |
| acetyl-CoA acetyltransferase | ZP_01011874 |
| acetyl-CoA acetyltransferase | ZP_00961105 |
| acetyl-CoA acetyltransferase | YP_426557 |
| acetyl-Coenzyme A acetyltransferase 3 | NP_694791 |
| acetyl-CoA acetyltransferase | YP_003153095 |
| Acetyl-CoA acetyltransferase | CCF95917 |
| acetyl-CoA acetyltransferase | ZP_07454459 |

TABLE 1C
Suitable homologues for the PhaB5 protein (acetoacetyl-CoA
reductase, from Zoogloea ramigera, EC No. 1.1.1.36, which
acts on acetoacetyl-CoA to produce 3-hydroxybutyryl CoA;
protein acc. no. P23238).

| | Protein Accession |
|---|---|
| Protein Name | No. |
| acetoacetyl-CoA reduetase | YP_002827755 |
| phaB gene product | YP_770184 |
| acetoacetyl-CoA reductase | ZP_08627619 |
| molybdopterin-guanine dinucleotide | ZP_01901796 |
| biosynthesis protein A | |
| acetoacetyl-CoA reductase | YP_006369576 |
| putative acetoacetyl-CoA reductase PhaB | ZP_09394630 |
| acetoacetyl-CoA reductase | YP_001352246 |
| acetoacetyl-CoA reductase | ZP_02467262 |
| acetoacetyl-CoA reductase | ZP_01985557 |

TABLE 1D
Suitable homologues for AccA protein (the alpha subunit of
Acetyl-CoA carboxylase from Escherichia coli, EC No.
6.4.1.2, which acts on acetyl-CoA to produce malonyl-CoA;
protein acc. no. AAC73296).

| | Protein Accession |
|---|---|
| Protein Name | No. |
| Acetyl-coenzyme A carboxylase carboxyl | WP_006893763 |
| transferase subunit alpha | |
| acetyl-CoA carboxylase, carboxyl | YP_114242 |
| transferase subunit alpha | |
| acetyl-CoA carboxylase, carboxyl | WP_005370898 |
| transferase, alpha subunit | |
| acetyl-CoA carboxylase alpha subunit | WP_007145523 |
| acetyl-coenzyine A carboxyl transferase | WP_009726998 |
| subunit alpha | |
| acetyl-coenzyme A carboxyl transferase | YP_006295989 |
| subunit alpha | |
| acetyl-coenzyme A carboxyl transferase | YP_006292159 |
| subunit alpha | |
| acetyl-CoA carboxylase, carboxyl | WP_008291216 |
| transferase, alpha subunit | |
| acetyl-CoA carboxylase carboxyltransferase | YP_275962 |
| subunit alpha | |

TABLE 1E
Suitable homologues for AccB protein (the BCCP (biotin
carboxyl carrier protein) subunit of Acetyl-CoA carboxylase
from Escherichia coli, EC No. 6.4.1.2, which acts on
acetyl-CoA to produce malonyl-CoA; protein acc. no.
AAC76287).

| Protein Name | Protein Accession No. |
|---|---|
| biotin carboxyl carrier protein | YP_544137 |
| acetyl-CoA carboxylase, biotin carboxyl | WP_006892078 |
| carrier protein | |
| acetyl-CoA carboxylase, biotin carboxyl | YP_113520 |
| carrier protein | |
| acetyl-CoA carboxylase, biotin carboxyl | WP_005368465 |
| carrier protein | |
| biotin carboxyl carrier protein of acetyl- | YP_004917568 |
| CoA carboxylase | |
| acetyl-CoA carboxylase biotin carboxyl | YP_006293649 |
| carrier protein | |
| acetyl-CoA carboxylase, biotin carboxyl | WP_008106026 |
| carrier protein | |
| Biotin carboxyl carrier protein of acetyl- | WP_008061392 |
| CoA carboxylase | |
| acetyl-CoA carboxylase, biotin carboxyl | YP_003049800 |
| carrier protein | |

TABLE 1F
Suitable homologues for the AccC protein (biotin carboxylase
subunit of Acetyl-CoA carboxylase from Escherichia coli,
EC No. 6.4.1.2, which acts on acetyl-CoA to produce
malonyl-CoA; protein acc. no. AAC76288).

| Protein Name | Protein Accession No. |
|---|---|
| biotin carboxylase/acetyl-coenzyme A | YP_544136 |
| carboxylase carboxyl transferase subunit | |
| alpha | |
| acetyl-CoA carboxylase, biotin | WP_006892077 |
| carboxylase | |
| acetyl-CoA carboxylase, biotin | YP_113521 |
| carboxylase | |
| acetyl-CoA carboxylase, biotin | WP_005368464 |
| carboxylase subunit | |
| acetyl-CoA carboxylase, biotin | YP_004512661 |
| carboxylase | |
| biotin carboxylase | WP_007143998 |
| biotin carboxylase | WP_009725888 |
| biotin carboxylase | YP_006293650 |
| biotin carboxylase | YP_006294632 |

TABLE 1G
Suitable homologues for the AccD protein (beta (carboxyltransferase)
subunit of Acetyl-CoA carboxylase from Escherichia coli, EC
No. 6.4.1.2, which acts on acetyl-CoA to produce malonyl-CoA;
protein acc. no. AAC75376).

| Protein Name | Protein Accession No. |
|---|---|
| AccD protein | AAU37781 |
| acetyl-CoA carboxylase subunit beta | YP_001347397 |
| acetyl-CoA carboxylase subunit beta | YP_005820031 |
| AccD | ABD19972 |
| acetyl-CoA carboxylase, carboxyl | YP_004116616 |
| transferase subunit beta | |
| Acetyl-coenzyme A carboxylase carboxyl | WP_009112050 |
| transferase subunit beta | |
| acetyl-CoA carboxylase subunit beta | WP_008910693 |
| acetyl-CoA carboxylase subunit beta | WP_006071327 |
| acetylCoA carboxylase, | WP_004586882 |
| carboxyltransferase subunit beta | |

TABLE 1H
Suitable homologues for the Mcr_{Ca}* protein (malonyl CoA
reductase (3-hydroxypropionate-forming), from Chloroflexus
aurantiacus, which acts on malonyl-CoA to produce
3-hydroxypropionate; protein acc. no. AAS20429).

| | Protein Accession |
|---|---|
| Protein Name | No. |
| short-chain dehydrogenase/reductase | YP_001636209 |
| short chain dehydrogenase/reductase | YP_002462600 |
| short-chain dehydrogenase/reductase | ZP_07684596 |
| dehydrogenase of unknown specificity | ZP_09692171.1\| |
| NAD-dependent epimerase/dehydratase | ZP_01039179 |
| short-chain alcohol dehydrogenase | YP_004863680 |
| oxidoreductase, short chain dehydrogenase/ | ZP_04957196 |
| reductase family | |
| short chain dehydrogenase | ZP_01626393 |
| short-chain dehydrogenase/reductase | ZP_05125944 |

TABLE 1I
Suitable homologues for the Mcr_{St} protein (Malonyl-CoA
reductase (malonate semialdehyde-forming), from Sulfolobus
tokodaii str. 7, EC No. 1.2.1.75, which acts on malonyl-CoA
to produce malonate semialdehyde; protein acc. no. BAB67276).

| | Protein Accession |
|---|---|
| Protein Name | No. |
| malonyl-/succinyl-CoA reductase | YP_004410014 |
| aspartate-semialdehyde dehydrogenase | YP_004459517 |
| aspartate-semialdehyde dehydrogenase | ZP_09704495 |
| aspartate-semialdehyde dehydrogenase | YP_002844727 |
| aspartate-semialdehyde dehydrogenase | YP_003401535 |
| aspartate-semialdehyde dehydrogenase | YP_003435562 |
| aspartate semialdehyde dehydrogenase | YP_004004235 |
| aspartate-semialdehyde dehydrogenase | YP_002461535 |
| aspartate-semialdehyde dehydrogenase | ZP_21643548 |

TABLE 1J
Suitable homologues for the MsaR_{St} protein (Malonic
semialdehyde reductase, from Sulfolobus tokodaii str. 7,
EC No. 1.1.1.298, which acts on malonate semialdehyde to
produce 3-hydroxypropionate; protein acc. no. BAK54608).

| | Protein Accession |
|---|---|
| Protein Name | No. |
| 3-hydroxyacyl-CoA dehydrogenase NAD- | YP_004458285 |
| binding protein | |
| malonate semialdehyde reductase | YP_004408885 |
| 3-hydroxyacyl-CoA dehydrogenase | YP_007865821 |
| 3-hydroxybutyryl-CoA dehydrogenase | YP_256228 |
| 3-hydroxyacyl-CoA dehydrogenas | YP_002832248 |
| 3-hydroxyacyl-CoA dehydrogenase | NP_070034 |
| 3-hydroxyacyl-CoA dehydrogenase NAD- | ZP_03264549 |
| binding | |
| 3-hydroxyacyl-CoA dehydrogenase | CCF36501 |
| 3-hydroxyacyl-CoA dehydrogenase NAD- | YP_005646018 |
| binding protein | |

TABLE 1K
Suitable homologues for the OrfZ protein (CoA transferase, from
Clostridium kluyveri DSM 555, EC No. 2.8.3.n, which acts on
3-hydroxypropionate to produce 3-hydroxypropionyl CoA; protein
acc. no. AAA92344)

| | Protein Accession |
|---|---|
| Protein Name | No. |
| 4-hydroxybutyrate coenzyme A transferase | YP_001396397 |
| acetyl-CoA hydrolase/transferase | ZP_05395303 |
| acetyl-CoA hydrolase/transferase | YP_001309226 |
| 4-hydroxybutyrate coenzyme A transferase | NP_781174 |
| 4-hydroxybutyrate coenzyme A transferase | ZP_05618453 |
| acetyl-CoA hydrolase/transferase | ZP_05634318 |
| 4-hydroxybutyrate coenzyme A transferase | ZP_00144049 |
| hypothetical protein ANASTE_01215 | ZP_02862002 |
| 4-hydroxybutyrate coenzyme A transferase | ZP_07455129 |

TABLE 1L
Suitable homologues for the AlkK protein (CoA ligase, a.k.a.
acyl CoA synthetase, from Pseudomonas putida, EC
No. 6.2.1.—, which acts on 3-hydroxypropionate to produce
3-hydroxypropionyl CoA; protein acc. no. CAB54055).

| | Protein Accession |
|---|---|
| Protein Name | No. |
| AMP-dependent synthetase and ligase | WP_009506504 |
| hypothetical protein | WP_004696716 |
| acyl-coa synthetase protein | YP_006029621 |
| medium-chain-fatty-acid--CoA ligase | WP_008641287 |
| acyl-CoA synthetase | WP_007607365 |
| medium-chain-fatty-acid--CoA ligase | YP_004981930 |
| AMP-dependent synthetase and ligase | WP_010683110 |
| AMP-dependent synthetase and ligase | YP_002499566 |
| AMP-dependent synthetase and ligase | YP_001769606 |

TABLE 1M
Suitable homologues for the DAR1 (GPD1) protein (Glycerol-
3-phosphate dehydrogenase (NAD+), from Saccharomyces
cerevisiae S288c, EC No. 1.1.1.8, which acts on dihydroxyacetone-
phosphate to produce sn-glycerol-3-phosphate; protein acc. no.
NP_010262).

| | Protein Accession |
|---|---|
| Protein Name | No. |
| hypothetical protein KAFR_0F02240 | XP_003957956 |
| K7_Gpd2p | GAA26268 |
| Glycerol-3-phosphate dehydrogenase | EFW94329 |
| glycerol-3-phosphate dehydrogenase | ABC17999 |
| PREDICTED: glycerol-3-phosphate | XP_004006414 |
| dehydrogenase [NAD(+)], cytoplasmic | |
| isoform 2 | |
| Glycerol-3-phosphate dehydrogenase | ENH63281 |
| [NAD+] | |
| ADR311Cp | NP_984407 |
| hypothetical protein CLUG_03347 | XP_002616106 |
| hypothetical protein Kpol_1037p2 | XP_001645264 |

TABLE 1N
Suitable homologues for the GpsA protein (Glycerol-3-
phosphate dehydrogenase (NADP+), from Rickettsia
prowazekii (strain Madrid E), EC No. 1.1.1.94, which
acts on dihydroxyacetone-phosphate to produce sn-glycerol-
3-phosphate; protein acc. no. NP_220823).

| | Protein Accession |
|---|---|
| Protein Name | No. |
| NAD(P)H-dependent glycerol-3-phosphate | YP_005391074 |
| dehydrogenase | |
| NAD(P)H-dependent glycerol-3-phosphate | WP_010423122 |
| dehydrogenase | |
| NAD(P)H-dependent glycerol-3-phosphate | YP_538395 |
| dehydrogenase | |
| NAD(P)H-dependent glycerol-3-phosphate | YP_001937693 |
| dehydrogenase | |
| Probable glycerol-3-phosphate | YP_001704643 |
| dehydrogenase 2 | |
| glycerol-3-phosphate dehydrogenase | WP_004982230 |
| [NAD(P)+] | |
| glycerol-3-phosphate dehydrogenase | WP_004679245 |
| (NAD(P)+) protein | |
| Glycerol-3-phosphate dehydrogenase | WP_012230519 |
| (NAD(P)+) 2 | |
| Glycerol-3-phosphate dehydrogenase | WP_006891779 |
| (NAD(P)+) | |

TABLE 1O
Suitable homologues for the GPP2 (HOR2) protein (Glycerol-3-
phosphatase, from Saccharomyces cerevisiae S288c,
EC No. 3.1.3.21, which acts on sn-glycerol-3-phosphate to
produce glycerol; protein acc. no. NP_010984).

| | Protein Accession |
|---|---|
| Protein Name | No. |
| unnamed protein product | CAA86169 |
| hypothetical protein KNAG_0L01510 | CCK72771 |
| hypothetical protein TPHA_0J00860 | XP_003687343 |
| potential DL-glycerol-3-phosphatase | XP_717809 |
| hypothetical protein | EME49670 |
| DOTSEDRAFT_164257 | |
| hypothetical protein MYCTH_2296323 | XP_003659378 |
| 2-deoxyglucose-6-phosphate phosphatase, | XP_002420967 |
| putative | |
| hor2p | EJS44022 |
| ZYRO0C08184p | XP_002496002 |

TABLE 1P
Suitable homologues for the DhaB1 protein (Glycerol
dehydratase large subunit, from Klebsiella pneumoniae,
EC No. 4.2.1.30, which acts on glycerol to produce
3-hydroxypropionaldehyde; protein acc. no. AAA74258).

| | Protein Accession |
|---|---|
| Protein Name | No. |
| DhaB | AAW50084 |
| propanediol dehydratase large subunit | WP_003441619 |
| glycerol dehydratase large subunit | WP_009624802 |
| propanediol dehydratase large subunit | WP_009730844 |
| propanediol dehydratase, large subunit | YP_795723 |
| B12-dependent diol dehydratase large | CAC82541 |
| subunit | |
| propanediol dehydratase large subunit | WP_003929110 |
| propanediol dehydratase, large subunit | YP_006455258 |
| glycerol dehydratase | YP_003989236 |

TABLE 1Q
Suitable homologues for the DhaB2 protein (Glycerol
dehydratase medium subunit, from Klebsiella pneumoniae,
EC No. 4.2.1.30, which acts on glycerol to produce
3-hydroxypropionaldehyde; protein acc. no. AAA74257).

| | Protein Accession |
|---|---|
| Protein Name | No. |
| hypothetical protein | WP_004098897 |
| coenzyme B12-dependent glycerol | NP_561846 |
| dehydrogenase medium subunit | |
| propanediol dehydratase medium subunit, | WP_001701970 |
| partial | |
| hypothetical protein | YP_003961987 |
| dehydratase medium subunit | YP_003994783 |
| dehydratase medium subunit | YP_003407459 |
| propanediol dehydratase large subunit | WP_003931871 |
| glycerol dehydratase | YP_004611539 |
| dehydratase medium subunit | WP_006299594 |

TABLE 1R
Suitable homologues for the DhaB3 protein (Glycerol
dehydratase small subunit, from Klebsiella pneumoniae,
EC No. 4.2.1.30, which acts on glycerol to produce
3-hydroxypropionaldehyde; protein acc. no. AAA74256).

| | Protein Accession |
|---|---|
| Protein Name | No. |
| glycerol dehydratase small subunit | ABA39278 |
| glycerol dehydratase small subunit | YP_006320551 |
| glycerol dehydratase, small subunit | WP_008821391 |
| propanediol dehydratase small subunit | WP_004105138 |
| propanediol utilization: dehydratase, small | WP_009201837 |
| subunit | |
| dehydratase small subunit | YP_004471781 |
| dehydratase small subunit | YP_004611538 |
| propanediol utilization: dehydratase, small | WP_003736322 |
| subunit | |
| hypothetical protein | WP_010739900 |

TABLE 1S
Suitable homologues for the GdrA protein (Chain A,
Glycerol Dehydratase Reactivase, from Klebsiella pneumoniae,
which acts on glycerol to produce 3-hydroxypropionaldehyde;
protein acc. no. AAA74255).

| | Protein Accession |
|---|---|
| Protein Name | No. |
| glycerol dehydratase large subunit | WP_007372194 |
| DhaF | AAP48652 |
| hypothetical protein | WP_004098901 |
| glycerol dehydratase reactivation factor, | YP_695622 |
| large subunit | |
| diol/glycerol dehydratase reactivating factor | WP_008725497 |
| large subunit | |
| glycerol dehydratase reactivation factor, | WP_003736323 |
| large subunit | |
| diol/glycerol dehydratase reactivating factor | YP_002892885 |
| large subunit | |
| Diol/glycerol dehydratase reactivating | WP_007062656 |
| factor large subunit | |
| hypothetical protein | WP_009267496 |

TABLE 1T
Suitable homologues for the GdrB protein (Chain B,
Glycerol Dehydratase Reactivase, from Klebsiella pneumoniae,
which acts on glycerol to produce 3-hydroxypropionaldehyde;
protein acc. no. 1NBW_B).

| | Protein Accession |
|---|---|
| Protein Name | No. |
| hypothetical protein | WP_005131414 |
| hypothetical protein WC1_03731 | EOQ21483 |
| putative diol/glycerol dehydratase | YP_006320602 |
| reactivating factor | |
| hypothetical protein | WP_003441632 |
| hypothetical protein GY4MC1_1865 | YP_003989240 |
| hypothetical protein Teth514_1949 | YP_001663563 |
| propanediol utilization diol dehydratase- | WP_008947524 |
| reactivating factor small chain | |
| propanediol utilization protein PduH | WP_009201835 |
| hypothetical protein | YP_003961984 |

TABLE 1U
Suitable homologues for the PuuC protein (3-Hydroxy-
propionaldehyde dehydrogenase (gamma-Glu-gamma-
aminobutyraldehyde dehydrogenase, NAD(P)H-dependent),
from Escherichia coli str. K-12 substr. MG1655, EC
No. 1.2.1.3, which acts on 3-hydroxypropionaldehyde to produce
3-hydroxypropionate; protein acc. no. NP_415816).

| | Protein Accession |
|---|---|
| Protein Name | No. |
| gamma-glutamyl-gamma- | YP_003363997 |
| aminobutyraldehyde dehydrogenase | |
| gamma-glutamyl-gamma- | WP_004860378 |
| aminobutyraldehyde dehydrogenase | |
| betaine aldehyde dehydrogenase | WP_001009084 |
| gamma-Glu-gamma-aminobutyraldehyde | YP_007405904 |
| dehydrogenase, NAD(P)H-dependent | |
| gamma-glutamyl-gamma- | YP_005093405 |
| aminobutyraldehyde dehydrogenase | |
| aldehyde dehydrogenase | YP_004993326 |
| NAD-dependent aldehyde dehydrogenase | WP_008086799 |
| aldehyde dehydrogenase | WP_008891845 |
| Gamma-glutamyl-gamma- | YP_003622830 |
| aminobutyraldehyde dehydrogenase | |

TABLE 1V
Suitable homologues for the PduP protein (CoA-acylating
3-hydroxypropionaldehyde dehydrogenase, from Salmonella
enterica subsp. enterica serovar Typhimurium str. LT2, EC
No. 1.2.1.—, which acts on 3-hydroxypropionaldehyde to produce
3-hydroxypropionyl CoA; protein acc. no. NP_460996).

| | Protein Accession |
|---|---|
| Protein Name | No. |
| hypothetical protein | WP_004105189 |
| propanediol utilization: CoA-dependent | YP_003365687 |
| propionaldehyde dehydrogenase | |
| CoA-dependent proprionaldehyde | YP_002383144 |
| dehydrogenase pduP | |
| CoA-dependent proprionaldehyde | YP_002556907 |
| dehydrogenase | |
| hypothetical protein | WP_008813236 |
| Aldehyde Dehydrogenase | YP_002892893 |
| aldehyde dehydrogenase family protein | WP_007372115 |
| CoA-dependent propionaldehyde | YP_849320 |
| dehydrogenase | |
| hypothetical protein | WP_010746532 |

TABLE 1W
Suitable homologues for the PhaC3/C1* protein (Polyhydroxyalkanoate
synthase fusion protein from Pseudomonas putida and Ralstonia eutropha
JMP134, EC No. 2.3.1.n, which acts on (R)-3-hydroxybutyryl-CoA or
3-hydroxypropionyl-CoA + [(R)-3-hydroxybutanoate-co-3-
hydroxypropionate]ₙ to produce [(R)-3-hydroxybutanoate-co-3-
hydroxypropionate]₍ₙ₊₁₎ + CoA and also acts on 3-
hydroxypropionyl-CoA + [3-hydroxypropionate]ₙ
to produce [3-hydroxypropionate]₍ₙ₊₁₎ + CoA.

| | Protein |
|---|---|
| Protein Name | Accession No. |
| Poly(R)-hydroxyalkanoic acid synthase, | YP_295561 |
| class I | |
| Poly(3-hydroxybutyrate)polymerase | YP_725940 |
| polyhydroxyalkanoic acid synthase | AAW65074 |
| polyhydroxyalkanoic acid synthase | YP_002005374 |
| Poly(R)-hydroxyalkanoic acid synthase, | YP_583508 |
| class I | |
| intracellular polyhydroxyalkanoate | ADM24646 |
| synthase | |
| Poly(3-hydroxyalkanoate) polymerase | ZP_00942942 |
| polyhydroxyalkanoic acid synthase | YP 003752369 |
| PhaC | AAF23364 |

TABLE 1X
Suitable homologues for the YqhD protein (succinic semialdehyde
reductase, from Escherichia coli K-12, EC No. 1.1.1.61,
which acts on 3-hydroxypropionaldehyde to produce 1,3-propanediol;
protein acc. no. NP_417484).

| | Protein |
|---|---|
| Protein Name | Accession No. |
| alcohol dehydrogenase yqhD | ZP_02900879 |
| alcohol dehydrogenase, NAD(P)-dependent | YP_002384050 |
| putative alcohol dehydrogenase | YP_003367010 |
| alcohol dehydrogenase YqhD | ZP_02667917 |
| putative alcohol dehydrogenase | YP_218095 |
| hypothetical protein ESA_00271 | YP_001436408 |
| iron-containing alcohol dehydrogenase | YP_003437606 |
| hypothetical protein CKO_04406 | YP_001455898 |
| alcohol dehydrogenase | ZP_03373496 |

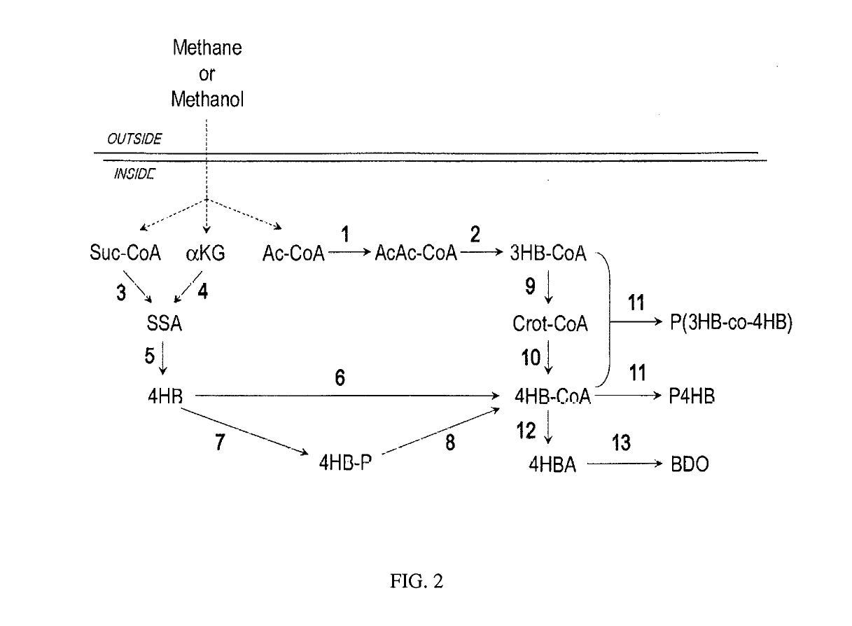

The genes cloned and/or assessed for host strains producing 4HB-containing PHA and 4-carbon chemicals were disclosed previously (International Pub. WO 2011/100601). Additional genes are presented below in Table 2A, along with the appropriate Enzyme Commission number (EC number) and references. As used herein, “heterologous” means from another host. The host can be the same or different species. FIG. 2 shows exemplary pathways for producing P4HB, P(3HB-co-4HB), and BDO.

TABLE 2A
Genes overexpressed or deleted in microbial host strains
producing 4HB-containing PHA and 4-carbon chemicals.

| Reaction | | | | |
|---|---|---|---|---|
| number | Gene | | EC | |
| (FIG. 2) | Name | Enzyme Name | Number | Accession No. |
| 9 | crt | 3-hydroxybutyryl- | 4.2.1.— | AAK80658 |
| | | CoA dehydratase | | |
| 10 | abfD | 4-Hydroxybutyryl- | 5.3.3.3 | CAB60035 |
| | | CoA dehydratase | 4.2.1.120 | |
| 12 | ald | Coenzyme A acylating | 1.2.1.10 | AAQ12068 |
| | | aldehyde dehydro- | | |
| | | genase | | |
| 13 | adh1 | Acetaldehyde dehydro- | 1.2.1.— | AY494991 |
| | | genase (acetylating) | | |

Proteins that may catalyze some of the biochemical reactions listed in Table 2A are provided in Tables 2B to 2E.

TABLE 2B
Suitable homologues for the Crt protein (3-hydroxybutyryl-
CoA dehydratase, from Clostridium acetobutylicum ATCC
824, EC No. 4.2.1.—, which acts on 3-hydroxybutyryl-CoA
to produce crotonyl-CoA; protein acc. no. AAK80658).

| | Protein |
|---|---|
| Protein Name | Accession No. |
| Enoyl-CoA hydratase/isomerase | YP_003844432 |
| 3-hydroxybutyryl-CoA dehydratase | YP_001884608 |
| 3-hydroxybutyryl-CoA dehydratase | WP_006940765 |
| Enoyl-CoA hydratase/isomerase | WP_007060131 |
| Enoyl-CoA hydratase/isomerase | YP_003153097 |
| Enoyl-CoA hydratase | YP_005847209 |
| enoyl-CoA hydratase/carnithine racemase | YP_007945423 |
| enoyl-CoA hydratase | WP_007872887 |
| 3-hydroxybutyryl-CoA dehydratase | YP_004883776 |

TABLE 2C
Suitable homologues for the AbfD protein (4-Hydroxybutyryl-
CoA dehydratase, from Clostridium aminobutyricum, EC Nos.
5.3.3.3 and 4.2.1.120, which acts on crotonyl-CoA to produce
4-hydroxybutyryl-CoA; protein acc. no. CAB60035).

| | Protein |
|---|---|
| Protein Name | Accession No. |
| Vinylacetyl-CoA delta-isomerase | WP_003423094 |
| gamma-aminobutyrate metabolism | WP_009014604 |
| dehydratase/isomerase | |
| gamma-aminobutyrate metabolism | YP_005014369 |
| dehydratase/isomerase | |
| aromatic ring hydroxylase | YP_006466005 |
| vinylacetyl-CoA delta-isomerase | YP_003702010 |
| 4-hydroxybutyryl-CoA dehydratase | YP_006721174 |
| 4-hydroxybutyryl-CoA dehydratase | YP_874977 |
| 4-hydroxyphenylacetate 3-monooxygenase | WP_007577713 |
| aromatic ring hydroxylase | YP_460766 |

TABLE 2D
Suitable homologues for the Ald protein (Coenzyme A acylating
aldehyde dehydrogenase, from Clostridium beijerinckii NCIMB
8052, EC No. 1.2.1.10, which acts on 4-hydroxybutyryl-CoA to
produce 4-hydroxybutyraldehyde; protein acc. no. AY494991).

| | Protein |
|---|---|
| Protein Name | Accession No. |
| butyraldehyde dehydrogenase | AAP42563 |
| coenzyme A acylating aldehyde | CAQ57983 |
| dehydrogenase | |
| ethanolamine utilization protein EutE | YP_001886323 |
| Aldehyde Dehydrogenase | WP_007505383 |
| aldehyde dehydrogenase | YP_006390854 |
| Aldehyde Dehydrogenase | YP_003822025 |
| aldehyde dehydrogenase | YP_003307836 |
| ethanolamine utilization protein EutE | WP_003736335 |
| aldehyde dehydrogenase | YP_958512 |

TABLE 2E
Suitable homologues for the Adh1 protein (acetaldehyde
dehydrogenase (acetylating), from Geobacillus thermoglucosidasius
strain M10EXG, EC No. 1.2.1.—, which acts on 4-hydroxybutyraldehyde
to produce 1,4-butanediol; protein acc. no. NP_149199).

| | Protein |
|---|---|
| Protein Name | Accession No. |
| aldehyde-alcohol dehydrogenase AdhE | YP_007456732 |
| bifunctional acetaldehyde-CoA/alcohol | WP_003447164 |
| dehydrogenase | |
| Aldehyde-alcohol dehydrogenase | WP_002780759 |
| Aldehyde-alcohol dehydrogenase | YP_005079865 |
| Aldehyde-alcohol dehydrogenase | WP_006303608 |
| aldehyde-alcohol dehydrogenase E, partial | AAM51642 |
| alcohol dehydrogenase, class IV | YP_007299947 |
| bifunctional acetaldehyde-CoA/alcohol | YP_002531871 |
| dehydrogenase | |
| bifunctional protein: acetaldehyde-CoA | WP_003253794 |
| dehydrogenase/alcohol dehydrogenase | |

TABLE 2F
Suitable homologues for the KgdM protein (alpha-ketoglutarate
decarboxylase, from Mycobacterium tuberculosis, EC No. 4.1.1.71,
which acts on alpha-ketoglutarate to produce succinate semialdehyde
and carbon dioxide; protein acc. no. NP_335730)

| | Protein |
|---|---|
| Protein Name | Accession No. |
| alpha-ketoglutarate decarboxylase | YP_001282558 |
| alpha-ketoglutarate decarboxylase | NP_854934 |
| 2-oxoglutarate dehydrogenase sucA | ZP_06454135 |
| 2-oxoglutarate dehydrogenase sucA | ZP_04980193 |
| alpha-ketoglutarate decarboxylase | NP_961470 |
| alpha-ketoglutarate decarboxylase Kgd | YP_001852457 |
| alpha-ketoglutarate decarboxylase | NP_301802 |
| alpha-ketoglutarate decarboxylase | ZP_05215780 |
| alpha-ketoglutarate decarboxylase | YP_001702133 |

TABLE 2G
Suitable homologues for the SucD protein (succinate semialdehyde
dehydrogenase, from Clostridium kluyveri, EC No. 1.2.1.76,
which acts on succinyl-CoA to produce succinate semialdehyde;
protein acc. no. YP_001396394)

| | Protein |
|---|---|
| Protein Name | Accession No. |
| CoA-dependent succinate semialdehyde | AAA92347 |
| dehydrogenase | |
| succinate-semialdehyde dehydrogenase | ZP_06559980 |
| [NAD(P)+] | |
| succinate-semialdehyde dehydrogenase | ZP_05401724 |
| [NAD(P)+] | |
| aldehyde-alcohol dehydrogenase family | ZP_07821123 |
| protein | |
| succinate-semialdehyde dehydrogenase | ZP_06983179 |
| [NAD(P)+] | |
| succinate-semialdehyde dehydrogenase | YP_001928839 |
| hypothetical protein CLOHYLEM_05349 | ZP_03778292 |
| succinate-semialdehyde dehydrogenase | YP_003994018 |
| [NAD(P)+] | |
| succinate-semialdehyde dehydrogenase | NP_904963 |

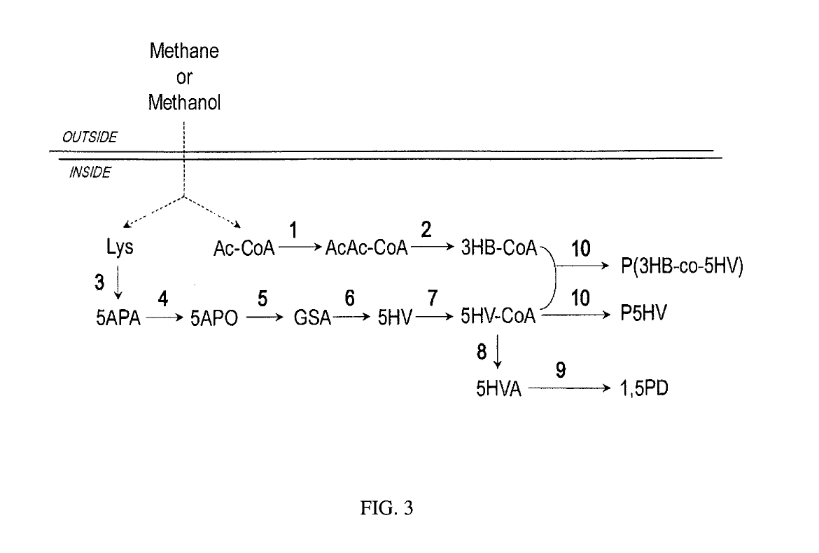

The genes cloned and/or assessed for host strains producing 5HV-containing PHA and 5-carbon chemicals, along with other proteins that may catalyze some of these biochemical reactions, were disclosed previously (US Patent Publication 2010/0168481). FIG. 3 shows exemplary pathways for producing P5HV, P(3HB-co-5HV), and 1,5PD.

### Suitable Extrachromosomal Vectors and Plasmids

A “vector,” as used herein, is an extrachromosomal replicon, such as a plasmid, phage, or cosmid, into which another DNA segment may be inserted so as to bring about the replication of the inserted segment. Vectors vary in copy number, depending on their origin of replication, and size. Vectors with different origins of replication can be propagated in the same microbial cell unless they are closely related such as e.g. pMB1 and ColE1.

Suitable vectors to express recombinant proteins in E. coli can constitute pUC vectors with a pMB1 origin of replication having 500-700 copies per cell, pBluescript vectors with a ColE1 origin of replication having 300-500 copies per cell, pBR322 and derivatives with a pMB1 origin of replication having 15-20 copies per cell, pACYC and derivatives with a p15A origin of replication having 10-12 copies per cell, and pSC101 and derivatives with a pSC101 origin of replication having about 5 copies per cell as described in the QIAGEN® Plasmid Purification Handbook (found on the world wide web at: kirshner.med.harvard.edu/files/protocols/QIAGEN_QIAGENPlasmidPurification_EN.pdf). A widely used vector is pSE380 that allows recombinant gene expression from an IPTG-inducible trc promoter (Invitrogen, La Jolla, Calif.).

Suitable vectors to express recombinant proteins in methylotrophic microorganisms include broad host-range vectors such as the low-copy number IncP1-based vectors pVK100 (Knauf and Nester, Plasmid 8:45-54 (1982)) and pLA2917 (Allen and Hanson, J. Bacteriol. 161:955-962 (1985)) with copy numbers between 5 to 7 and the higher copy number IncQ-based vectors pGSS8 (Windass et al., Nature 287:396-401 (1980)) and pAYC30 (Chistoserdov and Tsygankov, Plasmid 16:161-167 (1986)) with copy numbers between 10 to 12. Of particular practicality is the very small, broad host-range vector pBBR1 isolated from Bordetella bronchiseptica S87 (Antoine and Locht, Mol. Microbiol. 6(13):1785-1799 (1992)) as it does not belong to any of the broad host-range incompatibility groups IncP, IncQ or IncW and thus can be propagated together with other broad host-range vectors. Suitable derivatives from pBBR1 that contain antibiotic resistance markers include pBBR122 and pBHR1 that can be obtained from MoBiTec GmbH (Göttingen, Germany). Further derivatives of pBBR122 and pBHR1 containing other antibiotic resistance markers can be generated by genetic engineering by those skilled in the art.

Suitable Strategies and Expression Control Sequences for Recombinant Gene Expression

Strategies for achieving expression of recombinant genes in E. coli have been extensively described in the literature (Gross, Chimica Oggi 7(3):21-29 (1989); Olins and Lee, Cur. Op. Biotech. 4:520-525 (1993); Makrides, Microbiol. Rev. 60(3):512-538 (1996); Hannig and Makrides, Trends in Biotech. 16:54-60 (1998)). Expression control sequences can include constitutive and inducible promoters, transcription enhancers, transcription terminators, and the like which are well known in the art. Suitable promoters include, but are not limited to, P_{lac}, P_{tac}, P_{trc}, P_{R}, P_{L}, P_{phoA}, Pₐᵣₐ, P_{uspA}, P_{rpsU}, and P_{syn} (Rosenberg and Court, Ann. Rev. Genet. 13:319-353 (1979); Hawley and McClure, Nucl. Acids Res. 11 (8):2237-2255 (1983); Harley and Raynolds, Nucl. Acids Res. 15:2343-2361 (1987); also at the world wide web at ecocyc.org and partsregistry.org).

Strategies for achieving expression of recombinant genes in methylotrophic bacteria have also been described in the literature. Heterologous promoters, such as the artificial tac promoter described above and the E. coli trp promoter have been successfully used to express genes in M. methylotrophus (Byrom, In: Microbial Growth on C-1 Compounds (ed. Crawford and Hanson) pp. 221-223 (1984), Washington, D.C.: Am, Soc. Microbiol. Press). Other promoters such as the λP_{R} promoter and the promoter of the kanamycin resistance gene, Pₖₐₙ, were used to express the FLP recombinase of S. cerevisiae and the xylE gene from Pseudomonas putida, respectively (Abalakina et al., Appl. Microbiol. Biotechnol. 81:191-200 (2008)). The E. coli W3110 promoter of the Entner-Doudoroff pathway genes, P_{edd}, was also shown to work in M. methylotrophus (Ishikawa et al., Biosci. Biotechnol. Biochem. 72(10):2535-2542 (2008)). As several heterologous antibiotic markers derived from broad host-range plasmids are functional in methylotrophic bacteria, the promoters of the genes encoding enzymes conferring resistance towards e.g. ampicillin, tetracycline, chloramphenicol, streptomycin, or gentamycin can be used. As partial or complete genomic sequences have been established for several of these methane- and methanol-utilizing microorganisms, the promoters of endogenous genes can be used, e.g. the native promoter of the methanol dehydrogenase P_{mxaF} (Fitzgerald and Lidstrom, Biotechnol. Bioeng. 81(3):263-268 (2003); Belanger et al., FEMS Microbiol. Letters 231:197-204 (2004)) or the native promoter of the methane monooxygenase P_{pmoC} (Gilbert et al., Appl. Environ. Microbiol. 66(3):966-975 (2000)).

Exemplary promoters are:

P_{synA} (a.k.a. P_{syn1})
SEQ ID NO: 1
(5′-TTGACAGCTAGCTCAGTCCTAGGTATAATGCTAGC-3′),
P_{synC}
SEQ ID NO: 2
(5′-TTGACAGCTAGCTCAGTCCTAGGTACTGTGCTAGC-3′),
P_{synE}
SEQ ID NO: 3
(5′-TTTACAGCTAGCTCAGTCCTAGGTATTATGCTAGC-3′),
P_{synH}
SEQ ID NO: 4
(5′-CTGACAGCTAGCTCAGTCCTAGGTATAATGCTAGC-3′),
P_{synK}
SEQ ID NO: 5
(5′-TTTACGGCTAGCTCAGTCCTAGGTACAATGCTAGC-3′),
P_{synM}
SEQ ID NO: 6
(5′-TTGACAGCTAGCTCAGTCCTAGGGACTATGCTAGC-3′),
Pₓ
SEQ ID NO: 7
(5′-TCGCCAGTCTGGCCTGAACATGATATAAAAT-3′),
P_{uspA}
SEQ ID NO: 8
(5′-
AACCACTATCAATATATTCATGTCGAAAATTTGTTTATCTAACGAGTAAG
CAAGGCGGATTGACGGATCATCCGGGTCGCTATAAGGTAAGGATGGTCT
TAACACTGAATCCTTACGGCTGGGTTAGCCCCGCGCACGTAGTTCGCAG
GACGCGGGTGACGTAACGGCACAAGAAACG-3′),
P_{rpsU}
SEQ ID NO: 9
(5′-
ATGCGGGTTGATGTAAAACITTGTTCGCCCCTGGAGAAAGCCTCGTGTAT
ACTCCTCACCCTTATAAAAGTCCCTTTCAAAAAAGGCCGCGGTGCTTTAC
AAAGCAGCAGCAATTGCAGTAAAATTCCGCACCATTTTGAAATAAGCTG
GCGTTGATGCCAGCGGCAAAC-3′).
P^{synAF7}
SEQ ID NO: 10
(5′-TTGACAGCTAGCTCAGTCCTAGGTACAGTGCTAGC-3′)
P_{synAF3}
SEQ ID NO: 11
(5′-TTGACAGCTAGCTCAGTCCTAGGTACAATGCTAGC-3′)

Exemplary terminators are:

T_{trpL}
SEQ ID NO: 12
(5′-CTAATGAGCGGGCTTTTTTTTGAACAAAA-3′),
T₁₀₀₆
SEQ ID NO: 13
(5′-AAAAAAAAAAAACCCCGCTTCGGCGGGGTTTTTTTTTT-3′),
T_{rrnB1}
SEQ ID NO: 14
(5′-ATAAAACGAAAGGCTCAGTCGAAAGACTGGGCCTTTCGTTTT
AT-3′),
T_{rrnB2}
SEQ ID NO: 15
(5′-AGAAGGCCATCCTGACGGATGGCCTTTT-3′)

### Construction of Recombinant Hosts

Recombinant hosts containing the necessary genes that will encode the enzymatic pathway for the conversion of a carbon substrate to PHA biopolymers or C3, C4, and C5 biochemicals may be constructed using techniques well known in the art.

Methods of obtaining desired genes from a source organism (host) are common and well known in the art of molecular biology. Such methods are described in, for example, Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, Md. (1999). For example, if the sequence of the gene is known, the DNA may be amplified from genomic DNA using polymerase chain reaction (Mullis, U.S. Pat. No. 4,683,202) with primers specific to the gene of interest to obtain amounts of DNA suitable for ligation into appropriate vectors. Alternatively, the gene of interest may be chemically synthesized de novo in order to take into consideration the codon bias of the host organism to enhance heterologous protein expression. Expression control sequences such as promoters and transcription terminators can be attached to a gene of interest via polymerase chain reaction using engineered primers containing such sequences. Another way is to introduce the isolated gene into a vector already containing the necessary control sequences in the proper order by restriction endonuclcase digestion and ligation. One example of this latter approach is the BioBrick™ technology (www.biobricks.org) where multiple pieces of DNA can be sequentially assembled together in a standardized way by using the same two restriction sites.

In addition to using vectors, genes that are necessary for the enzymatic conversion of a carbon substrate to the desired products can be introduced into a host organism by integration into the chromosome using either a targeted or random approach. For targeted integration into a specific site on the chromosome, the method generally known as Red/ET recombineering is used as originally described by Datsenko and Wanner (Proc. Natl. Acad. Sci. USA, 2000, 97, 6640-6645). Another method for generating precise gene deletions and insertions in host strains involves the sacB gene that is used as a counterselectable marker for the positive selection of recombinant strains that have undergone defined genetic alterations leading to the loss of the marker (Steinmetz et al., Mol. Gen. Genet. 191:138-144 (1983); Reyrat et al., Infect Immun. 66(9): 4011-4017 (1998). Random integration into the chromosome involves using a mini-Tn5 transposon-mediated approach as described by Huisman et al. (U.S. Pat. Nos. 6,316,262 and 6,593,116). The TargeTron™ Gene Knockout System from Sigma-Aldrich (Oakville, ON, Canada) is another method for the rapid and specific disruption of genes in prokaryotic organisms. Introduction of recombinant DNA into the host organisms is accomplished for example using electroporation or conjugation (a.k.a. matings). These methods are well known to the artisan.

### Example 1

### Production of P3HP in Methylophilus methylotrophus from Methanol Using the Malonyl-CoA Reductase Metabolic Pathway

This example shows P3HP production from methanol as sole carbon source using the malonyl-CoA reductase route in engineered M. methylotrophus host cells (FIG. 1). The strains used in this example are listed in Table 3. Both strains were constructed using the well-known biotechnology tools and methods described above. Strain 1 lacked any of the recombinant genes, whereas strain 2 contained the engineered P3HP pathway genes.

TABLE 3
Strains used to produce P3HP from methanol carbon source.

| Strains | Genes Overexpressed |
|---|---|
| 1 | None (control strain) |
| 2 | T₁₀₀₆-P_{syn1}-phaC3/C1*-Tₜᵣₚ-P_{uspA}-mcr_{Ca}*-orfZ |

The strains were evaluated in a shake flask assay. The production medium consisted of 5.0 g/L (NH₄)₂SO₄, 0.097 g/L MgSO₄, 1.9 g/L K₂HPO₄, 1.38 g/L NaH₂PO₄.H₂O, 5.82 mg/L FeCl₃, 15.99 μg/L ZnSO₄, 17.53 μg/L MnSO₄.H₂O, 33.72 mg/L CaCl₂, 5 μg/L CuSO₄.5H₂O, 200 μM KOH and 2% (v/v) methanol. To examine production of P3HP, the strains were cultured three days in sterile tubes containing 3 mL, of production medium and appropriate antibiotics. Thereafter, 500 μL was removed from each tube and added to a sterile tube containing 4 mL of fresh production medium. The resulting 4.5 mL broths were cultured overnight. The next day, 1.3 mL was used to inoculate a sterile 250 mL flask containing 50 mL of production medium with appropriate antibiotics. The flasks were incubated at 37° C. with shaking for 5 hours and then shifted to 28° C. for 48 hours with shaking. Methanol was added to a final concentration of 1% into each flask after 24 hours of the 28° C. incubation period.

Thereafter, cultures from the flasks were analyzed for P3HP polymer content. At the end of the experiment, 1.5 mL of each culture broth was spun down at 12,000 rpm (13,523×g), washed twice with 0.2% NaCl solution, frozen at −80° C. for 1 hour, and lyophilized overnight. The next day, a measured amount of lyophilized cell pellet was added to a glass tube, followed by 3 mL of butanolysis reagent that consisted of a 1:3 volume mixture of 99.9% n-butanol and 4.0 M HCl in dioxane with 2 mg/mL diphenylmethane as internal standard. After capping the tubes, they were vortexed briefly and placed on a heat block set to 93° C. for 24 hours with periodic vortexing. Afterwards, the tubes were cooled down to room temperature before adding 3 mL deionized water. The tube was vortexed for approximately 10 s before spinning down at 600 rpm (Sorvall Legend RT benchtop centrifuge) for 2 min. 1 mL of the organic phase was pipetted into a GC vial, which was then analyzed by gas chromatography-flame ionization detection (GC-FID) (Agilent Technologies7890A). The quantity of PHA in the cell pellet was determined by comparing against standard curves for 3HP. The 3HP standard curve was generated by adding different amounts of poly-3-hydroxypropionate to separate butanolysis reactions.

The results for the two strains are shown in Table 4 and demonstrate that P3HP was produced from methanol as the sole carbon source.

TABLE 4
P3HP polymer production from microbial strains.

| | Biomass Titer | P3HP Titer |
|---|---|---|
| Strains | (g/L) | (g/L) |
| 1 | 0.683 | 0.000 |
| 2 | 0.767 | 0.008 |

### Example 2

### Production of P3HP in Methylophilus methylotrophus from Methanol Using the Glycerol Dehydratase Metabolic Pathway

This example shows P3HP production from methanol as sole carbon source using the glycerol dehydratase route in engineered M. methylotrophus host cells (FIG. 1). The strains used in this example are listed in Table 5. Both strains are constructed using the well-known biotechnology tools and methods described above. Strain 1 lacks any of the recombinant genes, whereas strain 3 contains the engineered P3HP pathway genes.

TABLE 5
Strains used to produce P3HP from methanol carbon source.

| Strains | Genes Overexpressed |
|---|---|
| 1 | None (control strain) |
| 3 | P_{ompA}-dhaB1-dhaB2-dhaB3-gdrA-gdrB-T_{rrnB1}; P_{uspA}- |
| | PhaC3/C1*-T_{rrnB1}; P_{rpsU}-orfZ-puuC-T_{rrnB1}; P_{syn1}-pduP- |
| | DAR1-GPP2 |

The strains are evaluated in a shake flask assay. The production medium is the same as the one listed in Example 1 with the exception that 1 μM vitamin B12 is added to the medium. Growth and determination of biomass and P3HP titers are performed as outlined in Example 1. Control strain 1 is expected to be unable to produce P3HP, whereas strain 3 is anticipated to produce P3HP owing to the engineered pathway genes.

### Example 3

### Production of P(3HB-Co-3HP) in Methylophilus methylotrophus from Methanol

This example shows P(3HB-co-3HP) production from methanol as sole carbon source using either the malonyl-CoA reductase or the glycerol dehydratase metabolic pathways in engineered M. methylotrophus host cells (FIG. 1). The strains used in this example are listed in Table 6. Both strains were constructed using the well-known biotechnology tools and methods described above. Strain 1 lacked any of the recombinant genes, whereas strain 4 contained different pathway genes enabling production of P(3HB-co-3HP) copolymer.

TABLE 6
Strains used to produce P(3HB-co-
3HP) from methanol carbon source.

| Strains | Genes Overexpressed |
|---|---|
| 1 | None (control strain) |
| 4 | T₁₀₀₆-P_{syn1}-PhaC3/C1*-Tₜᵣₚ-P_{uspA}-mcr_{Ca}*-orfZ; P_{uspA}-phaA5- |
| | phaB5 |

The strains were evaluated in a shake flask assay. The production medium was the same as the one listed in Example 1 and culture was performed as outlined in Example 1 except the flask culture started with 250 mL flask containing 30 mL of production medium and 300 μL of 100% methanol and 600 μL of 50×E0 buffer that consisted of 375 g/L K₂HPO₄.3H₂O, 185 g/L KH₂PO₄, and 181 g/L Na₂HPO₄ were added into the culture after 24 hours incubation at 28° C.

Determination of biomass and the contents of 3HB and 3HP in the polymer were performed as outlined in Example 1. The quantity of PHA in the cell pellet was determined by comparing against standard curves for 3HB and 3HP (for P(3HB-co-3HP) analysis). The 3HB standard curve was generated by adding different amounts of 99% ethyl 3-hydroxybutyrate to separate butanolysis reactions. The 3HP standard curve was generated by adding different amounts of poly-3-hydroxybutyrate to separate butanolysis reactions.

The results for the two strains are shown in Table 7 and demonstrated that P(3HB-co-3HP) copolymer was produced from methanol as the sole carbon source.

TABLE 7
P(3HB-co-3HP) copolymer production from microbial strains.

| | Biomass Titer | P(3HB-co-HP) Titer | 3HB Titer | 3HP Titer |
|---|---|---|---|---|
| Strains | (g/L) | (g/L) | (g/L) | (g/L) |
| 1 | 4.301 | 0.000 | 0.000 | 0.000 |
| 2 | 5.118 | 0.024 | 0.014 | 0.010 |

Using a methylotrophic microorganism such as e.g. Methylobacterium extorquens AM1, which is known to naturally produce P3HB homopolymer (Korotkova and Lidstrom, J. Bacteriol. 183(3):1038-1046 (2001)), the genetic engineering would not need to include the phaA and phaB genes encoding the enzymes that produce the 3HB-CoA precursor molecule for the production of the P(3HB-co-3HP) copolymer. However, unwanted endogenous PHA biosynthesis and degradation genes such as PHA synthases and depolymerases would need to be removed from the host organism.

### Example 4

### Production of PDO in Methylophilus methylotrophus from Methanol

This example shows PDO production from methanol as sole carbon source using either the malonyl-CoA reductase or the glycerol dehydratase metabolic pathways in engineered M. methylotrophus host cells (FIG. 1). The strains used in this example are listed in Table 8. All strains are constructed using the well-known biotechnology tools and methods described above. Strain 1 lacks any of the recombinant genes, whereas strains 6 and 7 contain the engineered pathway genes.

TABLE 8
Strains used to produce PDO from methanol carbon source.

| Strains | Genes Overexpressed |
|---|---|
| 1 | None (control strain) |
| 6 | T₁₀₀₆-P_{syn1}-yqhD-Tₜᵣₚ- P_{uspA}-mcr_{Ca}*-puuC |
| 7 | P_{ompA}-dhaB1-dhaB2-dhaB3-gdrA-gdrB-T_{rrnB1}; P_{uspA}-yqhD- |
| | T_{rrnB1}; P_{syn1}-DAR1-GPP2 |

The strains are evaluated in a shake flask assay. The production medium is the same as the one listed in Example 1 with the exception that 30 μM vitamin B12 is added to the medium for strain 7. Growth is performed as outlined in Example 1. The concentration of PDO is measured by GC/MS. Analyses are performed using standard techniques and materials available to one of skill in the art of GC/MS. One suitable method utilized a Hewlett Packard 5890 Series II gas chromatograph coupled to a Hewlett Packard 5971 Series mass selective detector (EI) and a HP-INNOWax column (30 m length, 0.25 mm i.d., 0.25 micron film thickness). The retention time and mass spectrum of PDO generated were compared to that of authentic PDO (m/e: 57, 58). Control strain 1 is expected to be unable to produce PDO, whereas strains 6 and 7 are anticipated to produce PDO owing to the engineered pathway genes.

### Example 5

### Production of P4HB in Methylophilus methylotrophus from Methanol

This example shows P4HB production from methanol as sole carbon source in engineered M. methylotrophus host cells (FIG. 2). The strains used in this example are listed in Table 9. All strains are constructed using the well-known biotechnology tools and methods described above. Strain 1 lacks any of the recombinant genes, whereas strain 8 contains the engineered P4HB pathway genes.

TABLE 9
Strains used to produce P4HB from methanol carbon source.

| Strains | Genes Overexpressed |
|---|---|
| 1 | None (control strain) |
| 8 | P_{uspA}-phaC3/C1*-ssaR*, P_{rpsU}-orfZ, Pₜₑₜ-sucD* |

The strains are evaluated in a shake flask assay. The production medium, cell growth, and determination of biomass are the same as described in Example 1. Determination of P4HB titers are performed as follows: a measured amount of lyophilized cell pellet was added to a glass tube, followed by 3 mL of butanolysis reagent that consists of an equal volume mixture of 99.9% n-butanol and 4.0 N HCl in dioxane with 2 mg/mL diphenylmethane as internal standard. After capping the tubes, they are vortexed briefly and placed on a heat block set to 93° C. for six hours with periodic vortexing. Afterwards, the tube is cooled down to room temperature before adding 3 mL distilled water. The tube is vortexed for approximately 10 s before spinning down at 620 rpm (Sorvall Legend RT benchtop centrifuge) for 2 min. 1 mL of the organic phase is pipetted into a GC vial, which is then analyzed by gas chromatography-flame ionization detection (GC-FID) (Hewlett-Packard 5890 Series II). The quantity of PHA in the cell pellet is determined by comparing against a standard curve for 4HB (for P4HB analysis). The 4HB standard curve is generated by adding different amounts of a 10% solution of γ-butyrolactone (GBL) in butanol to separate butanolysis reactions.

Control strain 1 is expected to be unable to produce P4HB, whereas strain 8 is anticipated to produce P4HB owing to the engineered pathway genes.

### Example 6

### Production of P(3HB-Co-4HB) in Methylophilus methylotrophus from Methanol

This example shows P(3HB-co-4HB) production from methanol as sole carbon source in engineered M. methylotrophus host cells (FIG. 2). The strains used in this example are listed in Table 10. All strains are constructed using the well-known biotechnology tools and methods described above. Strain 1 lacks any of the recombinant genes, whereas strains 9 and 10 contain the engineered pathway genes.

TABLE 10
Strains used to produce P(3HB-co-
4HB) from methanol carbon source.

| Strains | Genes Overexpressed |
|---|---|
| 1 | None (control strain) |
| 9 | P_{uspA}-phaC3/C1*-ssaR*; P_{rpsU}-orfZ, Pₜₑₜ-sucD*; P_{uspA}- |
| | phaA5-phaB5 |
| 10 | P_{uspA}-phaC3/C1*; P_{uspA}-phaA5-phaB5; P_{rpsU}-crt-abfD |

The strains are evaluated in a shake flask assay. The production medium, cell growth, and determination of biomass are the same as described in Example 1, whereas determination of 3HB and 4HB titers are performed as described in Examples 3 and 5.

Control strain 1 is expected to be unable to produce P(3HB-co-4HB), whereas strains 9 and 10 are anticipated to produce P(3HB-co-4HB) owing to the engineered pathway genes.

Using a methylotrophic microorganism such as e.g. Methylobacterium extorquens AM1, which is known to naturally produce P3HB homopolymer (Korotkova and Lidstrom, J. Bacteriol. 183(3):1038-1046 (2001)), the genetic engineering would not need to include the phaA and phaB genes encoding the enzymes that produce the 3HB-CoA precursor molecule for the production of the P(3HB-co-4HB) copolymer.

However, unwanted endogenous PHA biosynthesis and degradation genes such as PHA synthases and depolymerases would need to be removed from the host organism.

### Example 7

### Production of BDO in Methylophilus methylotrophus from Methanol (Prophetic Example)

This example shows BDO production from methanol as sole carbon source in engineered M. methylotrophus host cells (FIG. 2). The strains used in this example are listed in Table 11. All strains are constructed using the well-known biotechnology tools and methods described above. Strain 1 lacks any of the recombinant genes, whereas strains 11 and 12 contain the engineered pathway genes.

TABLE 11
Strains used to produce BDO from methanol carbon source.

| Strains | Genes Qverexpressed |
|---|---|
| 1 | None (control strain) |
| 11 | P_{uspA}-sucD*-ssaR*; P_{rpsU}-orfZ, Pₜₑₜ-adh-adh1 |
| 12 | P_{uspA}-phaA5-phaB5; P_{rpsU}-crt-abfD; Pₜₑₜ-adh-adh1 |

The strains are evaluated in a shake flask assay. The production medium and cell growth is the same as described in Example 1. BDO in cell culture samples is derivatized by silylation and quantitatively analyzed by GC/MS as described by Simonov et al. (J. Anal. Chem. 59:965-971 (2004)).

Control strain 1 is expected to be unable to produce BDO, whereas strains 11 and 12 are anticipated to produce BDO owing to the engineered pathway genes.

### Example 8

### Production of P5HV in Methylophilus methylotrophus from Methanol

This example shows P5HV production from methanol as sole carbon source in engineered M. methylotrophus host cells (FIG. 3). The strains used in this example are listed in Table 12. All strains are constructed using the well-known biotechnology tools and methods described above. Strain 1 lacks any of the recombinant genes, whereas strain 13 contains the engineered P5HV pathway genes.

TABLE 12
Strains used to produce P5HV from methanol carbon source.

| Strains | Genes Overexpressed |
|---|---|
| 1 | None (control strain) |
| 13 | P_{rpsU}-ssaR*; P_{uspA}-phaC3/C1*-orfZ; P_{ompA}-davB-davA-davT |

The strains are evaluated in a shake flask assay. The production medium, cell growth, and determination of biomass are as described in Example 1. Determination of P5HV titers are performed as follows: a measured amount of lyophilized cell pellet is added to a glass tube, followed by 3 mL of butanolysis reagent that consists of an equal volume mixture of 99.9% n-butanol and 4.0 N HCl in dioxane with 2 mg/mL diphenylmethane as internal standard. After capping the tubes, they are vortexed briefly and placed on a heat block set to 93° C. for 6 hours with periodic vortexing. Afterwards, the tubes are cooled down to room temperature before adding 3 mL distilled water. The tubes are vortexed for approximately 10 s before spinning down at 620 rpm (Sorvall Legend RT benchtop centrifuge) for 2 min. 1 mL of the organic phase is pipetted into a GC vial, which is then analyzed by gas chromatography-flame ionization detection (GC-FID) (Hewlett-Packard 5890 Series II). The quantity of P(5HV) homopolymer in the cell pellet is determined by comparing against standard curves that are made by adding defined amounts of delta-valerolactone (DVL) in separate butanolysis reactions.

Control strain 1 is expected to be unable to produce P5HV, whereas strain 13 is anticipated to produce P5HV owing to the engineered pathway genes.

### Example 9

### Production of P(3HB-Co-5HV) in Methylophilus methylotrophus from Methanol (Prophetic Example)

This example shows P(3HB-co-5HV) production from methanol as sole carbon source in engineered M. methylotrophus host cells (FIG. 3). The strains used in this example are listed in Table 13. All strains are constructed using the well-known biotechnology tools and methods described above. Strain 1 lacks any of the recombinant genes, whereas strain 14 contains the engineered P(3HB-co-5HV) pathway genes.

TABLE 13
Strains used to produce P(3HB-co-
5HV) from methanol carbon source.

| Strains | Genes Overexpressed |
|---|---|
| 1 | None (control strain) |
| 14 | P_{rpsU}-ssaR*; P_{uspA}-phaC3/C1*-orfZ,; P_{ompA}-davB-davA-davT; |
| | P_{uspA}-phaA5-phaB5 |

The strains are evaluated in a shake flask assay. The production medium, cell growth, and determination of biomass are the same as described in Example 1, whereas determination of 3HB and 5HV titers are performed as described in Examples 3 and 8.

Control strain 1 is expected to be unable to produce P(3HB-co-5HV), whereas strain 14 is anticipated to produce P(3HB-co-5HV) owing to the engineered pathway genes.

Using a methylotrophic microorganism such as e.g. Methylobacterium extorquens AM1, which is known to naturally produce P3HB homopolymer (Korotkova and Lidstrom, J. Bacteriol. 183(3): 1038-1046 (2001)), the genetic engineering would not need to include the phaA and phaB genes encoding the enzymes that produce the 3HB-CoA precursor molecule for the production of the P(3HB-co-5HV) copolymer.

However, unwanted endogenous PHA biosynthesis and degradation genes such as PHA synthases and depolymerases would need to be removed from the host organism.

### Example 10

### Production of 1,5PD in Methylophilus methylotrophus from Methanol

This example shows 1,5PD production from methanol as sole carbon source in engineered M. methylotrophus host cells (FIG. 3). The strains used in this example are listed in Table 14. All strains are constructed using the well-known biotechnology tools and methods described above. Strain 1 lacks any of the recombinant genes, whereas strain 15 contains the engineered 1,5PD pathway genes.

TABLE 14
Strains used to produce 1,5-PD from methanol carbon source.

| Strains | Genes Overexpressed |
|---|---|
| 1 | None (control strain) |
| 15 | P_{rpsU}-ssaR*; P_{uspA}-pduP-dhaT-orfZ; P_{ompA}-davB-davA-davT |

The strains are evaluated in a shake flask assay. The production medium and cell growth is the same as described in Example 1. 1,5PD in cell culture samples is quantitatively analyzed by GC/MS as described by Farmer et al. (US Patent Pub. 2010/0168481).

Control strain 1 is expected to be unable to produce 1,5-PD, whereas strain 15 is anticipated to produce 1,5-PD owing to the engineered pathway genes.

### Example 11

### Production of P3HP in Methylocystis hirsuta from Methane

This example shows P3HP production from methane as sole carbon source using the malonyl-CoA reductase or the glycerol dehydratase routes in engineered Methylocystis hirsuta host cells (FIG. 1). The strains used in this example are listed in Table 15. All strains are constructed using the well-known biotechnology tools and methods described above. All strains have the endogenous PHB biosynthesis (phaA, phaB, phaC1 and phaC2) and depolymerase genes (depA and depB) inactivated. Strain 16 lacks any of the recombinant genes, whereas strains 17 and 18 contain the engineered pathway genes.

TABLE 15
Strains used to produce P3HP from methanol carbon source.

| | Relevant Host | |
|---|---|---|
| Strains | Gene Inactivation | Genes Overexpressed |
| 16 | phaA, phaB, phaC1, | None (control strain) |
| | phaC2, depA, depB | |
| 17 | phaA, phaB, phaC1, | T₁₀₀₆-P_{syn1}-phaC3/C1*- Tₜᵣₚ- P_{uspA}- |
| | phaC2, depA, depB | mcr_{Ca}*-orfZ |
| 18 | phaA, phaB, phaC1, | P_{ompA}-dhaB1-dhaB2-dhaB3-gdrA-gdrB- |
| | phaC2, depA, depB | T_{rrnB1}; P_{uspA}-phaC3/C1*-T_{rrnB1}; P_{rpsU}- |
| | | orfZ-puuC-T_{rrnB1}; P_{syn1}-pduP-DAR1- |
| | | GPP2 |

Methane is used as sole carbon source at pH 7 and 30° C. for cell growth and product accumulation. The composition of the culture medium is as follows (g/L): (NH₄)₂SO₄ (1.75); MgSO₄.7H₂O (0.1); CaCl₂.2H₂O (0.02); KH₂PO₄ (0.68); Na₂HPO₄.12H₂O (6.14); FeSO₄.7H₂O (4 g/50 cc) and trace elements (mg/L) made of MnSO₄′7H₂O (5); ZnSO₄.7T₂O (1.5); Na₂MoO₄.2Hf₂O (0.04); CuSO₄.5H₂O (0.04); CoCl₂.6H₂O (0.6) and H₃BO₃ (0.2). For strain 18, 30 μM vitamin B12 is added to the medium. Cell growth and inoculum preparation for the bubble column reactor is as described previously by Rahnama et al. (Biochem. Engin. J. 65; 51-56 (2012)). Briefly, plates are gassed with a natural gas/air mixture (1:1, v/v) in a sealed desiccator. The gas phase is refilled every 12 h with the same gas mixture. The cultivation of cells is carried out at 30° C. for about 18 days. After this stage, one loop of the germinated colonies is cultivated in the mineral medium containing 1% (v/v) methanol in a shake flask. The cultivation in shake flasks is incubated at 30° C. and 200 rpm for 72 h to prepare the required inocula for a bubble bioreactor. P3HP production occurs in a 1 L bubble column bioreactor.

Natural gas and air streams are introduced through separate lines, mixed at the bottom of the reactor, and fed into the column by a sparger. To prevent evaporation, a condenser is installed at the top of the column. For all experiments, reactor temperature and pH are adjusted at 30° C. and 7.0, respectively, by a heat controllable water bath and 1.0 N HCl/NaOH solution. 20 mL of the shake-flask culture is inoculated into 180 mL of the fresh medium and incubated at 30° C. under continuous aeration of a natural gas/air mixture in a bubble-column bioreactor. All cultivations are performed in two stages as follows. Cells are grown in liquid medium under a natural gas/air mixture in the bubble column bioreactor at 30° C. In the second stage, cells are harvested by centrifugation at 5000 rpm for 20 min and the pellets are resuspended in the medium with nitrogen deficiency.

Determination of biomass and P3HP titers are performed as outlined in Example 1. Control strain 16 is expected to be unable to produce P3HP, whereas strains 17 and 18 are anticipated to produce P3HP owing to the engineered pathway genes.

### Example 12

### Production of P(3HB-Co-3HP) in Methylocystis hirsuta from Methane

This example shows P(3HB-co-3HP) production from methane as sole carbon source using the malonyl-CoA reductase or the glycerol dehydratase routes in engineered Methylocystis hirsuta host cells (FIG. 1). The strains used in this example are listed in Table 16. All strains are constructed using the well-known biotechnology tools and methods described above. All strains have the endogenous PHA synthase (phaC1 and phaC2) and depolymerase genes (depA and depB) inactivated, but retain the phaA and phaB genes for copolymer production. Strain 19 lacks all of the recombinant genes, whereas strains 20 and 21 contain the engineered pathway genes.

TABLE 16
Strains used to produce P(3HB-co-
3HP) from methanol carbon source.

| | Relevant Host | |
|---|---|---|
| Strains | Gene Inactivation | Genes Overexpressed |
| 19 | phaC1, phaC2, | None (control strain) |
| | depA, depB | |
| 20 | phaC1, phaC2, | T₁₀₀₆-P_{syn1}-phaC3/C1*-Tₜᵣₚ-P_{uspA}-mcr_{Ca}*- |
| | depA, depB | orfZ |
| 21 | phaC1, phaC2, | P_{ompA}-dhaB1-dhaB2-dhaB3-gdrA-gdrB- |
| | depA, depB | T_{rrnB1}; P_{uspA}-phaC3/C1*-T_{rrnB1}; P_{rpsU}-orfZ- |
| | | puuC-T_{rrnB1}; P_{syn1}-pduP-DAR1-GPP2 |

The strains are grown and evaluated as described in Example 11. The growth medium of strain 21 also contains 30 μM vitamin B12. The determination of 3HB and 3HP titers of the P(3HB-co-3HP) copolymer are performed as described in Examples 3 and 1. Control strain 19 is expected to be unable to produce P(3HB-co-3HP), whereas strains 20 and 21 are anticipated to produce P(3HB-co-3HP) owing to the engineered pathway genes.

### Example 13

### Production of PDO in Methylocystis hirsuta from Methane

This example shows PDO production from methane as sole carbon source using the malonyl-CoA reductase or the glycerol dehydratase routes in engineered Methylocystis hirsuta host cells (FIG. 1). The strains used in this example are listed in Table 17. All strains are constructed using the well-known biotechnology tools and methods described above. All strains have the endogenous PHB biosynthesis (phaA, phaB, phaC1 and phaC2) and depolymerase genes (depA and depB) inactivated. Strain 16 lacks all of the recombinant genes, whereas strains 22 and 23 contain the engineered pathway genes.

TABLE 17
Strains used to produce PDO from methanol carbon source.

| | Relevant Host | |
|---|---|---|
| Strains | Gene Inactivation | Genes Overexpressed |
| 16 | phaA , phaB, phaC1, | None (control strain) |
| | phaC2, depA, depB | |
| 22 | phaA, phaB, phaC1, | T₁₀₀₆-P_{syn1}-yqhD-Tₜᵣₚ- P_{uspA}-mcr_{Ca}*- |
| | phaC2, depA, depB | puuC |
| 23 | phaA, phaB, phaC1, | P_{ompA}-dhaB1-dhaB2-dhaB3-gdrA-gdrB- |
| | phaC2, depA, depB | T_{rrnB1}; P_{uspA}-yqhD-T_{rrnB1}; P_{syn1}-DAR1- |
| | | GPP2 |

The strains are grown and evaluated as described in Example 11. The growth medium of strain 23 also contains 30 μM vitamin B12. The concentration of PDO is measured by GC/MS as described in Example 4. Control strain 16 is expected to be unable to produce PDO, whereas strains 22 and 23 are anticipated to produce PDO owing to the engineered pathway genes.

### Example 14

### Production of P4HB in Methylocystis hirsuta from Methane

This example shows P4HB production from methane as sole carbon source in engineered Methylocystis hirsuta host cells (FIG. 2). The strains used in this example are listed in Table 18. All strains are constructed using the well-known biotechnology tools and methods described above. All strains have the endogenous PHB biosynthesis (phaA, phaB, phaC1 and phaC2) and depolymerase genes (depA and depB) inactivated. Strain 16 lacks all of the recombinant genes, whereas strain 24 contains the engineered pathway genes.

TABLE 18
Strains used to produce P4HB from methanol carbon source.

| | Relevant Host | |
|---|---|---|
| Strains | Gene Inactivation | Genes Overexpressed |
| 16 | phaA, phaB, phaC1, | None (control strain) |
| | phaC2, depA, depB | |
| 24 | phaA, phaB, phaC1, | P_{uspA}-phaC3/C1*-ssaR*, P_{rpsU}-orfZ, |
| | phaC2, depA, depB | Pₜₑₜ-sucD* |

The strains are grown and evaluated as described in Example 11. Determination of P4HB titers are as described in Example 5. Control strain 16 is expected to be unable to produce P4HB, whereas strain 24 is anticipated to produce P4HB owing to the engineered pathway genes.

### Example 15

### Production of P(3HB-Co-4HB) in Methylocystis hirsuta from Methane

This example shows P(3HB-co-4HB) production from methane as sole carbon source in engineered Methylocystis hirsuta host cells (FIG. 2). The strains used in this example are listed in Table 19, All strains are constructed using the well-known biotechnology tools and methods described above. All strains have the endogenous PHA synthase (phaC1 and phaC2) and depolymerase genes (depA and depB) inactivated, but retain the phaA and phaB genes for copolymer production. Strain 19 lacks all of the recombinant genes, whereas strains 25 and 26 contain the engineered pathway genes.

TABLE 19
Strains used to produce P(3HB-co-
4HB) from methanol carbon source.

| | Relevant Host | |
|---|---|---|
| Strains | Gene Inactivation | Genes Overexpressed |
| 19 | phaC1, phaC2, | None (control strain) |
| | depA, depB | |
| 25 | phaC1, phaC2, | P_{uspA}-phaC3/C1*-ssaR*, P_{rpsU}-orfZ, |
| | depA, depB | Pₜₑₜ-sucD |
| 26 | phaC1, phaC2, | P_{uspA}-phaC3/C1*; P_{rpsU}-crt-abfD |
| | depA, depB | |

The strains are grown and evaluated as described in Example 11. The determination of 3HB and 4HB titers are performed as described in Examples 3 and 5. Control strain 19 is expected to be unable to produce P(3HB-co-4HB), whereas strains 25 and 26 are anticipated to produce P(3HB-co-4HB) owing to the engineered pathway genes.

### Example 16

### Production of BDO in Methylocystis hirsuta from Methane

This example shows BDO production from methane as sole carbon source in engineered Methylocystis hirsuta host cells (FIG. 2). The strains used in this example are listed in Table 20, All strains are constructed using the well-known biotechnology tools and methods described above. All strains have the endogenous PHB biosynthesis (phaA, phaB, phaC1 and phaC2) and depolymerase genes (depA and depB) inactivated, Strain 16 lacks all of the recombinant genes, whereas strains 27 and 28 contain the engineered pathway genes.

TABLE 20
Strains used to produce BDO from methanol carbon source.

| | Relevant Host | |
|---|---|---|
| Strains | Gene Inactivation | Genes Overexpressed |
| 16 | phaA, phaB, phaC1, | None (control strain) |
| | phaC2, depA, depB | |
| 27 | phaA, phaB, phaC1, | P_{uspA}-sucD*-ssaR*; P_{rpsU}-orfZ, |
| | phaC2, depA, depB | Pₜₑₜ-adh-adh1 |
| 28 | phaA, phaB, phaC1, | P_{uspA}-phaA5-phaB5; P_{rpsU}-crt-abfD; |
| | phaC2, depA, depB | Pₜₑₜ-adh-adh1 |

The strains are grown and evaluated as described in Example 11. BDO in cell culture samples is determined as described in Example 7. Control strain 16 is expected to be unable to produce BDO, whereas strains 27 and 28 are anticipated to produce BDO owing to the engineered pathway genes.

### Example 17

### Production of P5HV in Methylocystis hirsuta from Methane

This example shows P5HV production from methane as sole carbon source in engineered Methylocystis hirsuta host cells (FIG. 3). The strains used in this example are listed in Table 21. All strains are constructed using the well-known biotechnology tools and methods described above. All strains have the endogenous PHB biosynthesis (phaA, phaB, phaC1 and phaC2) and depolymerase genes (depA and depB) inactivated. Strain 16 lacks all of the recombinant genes, whereas strain 29 contains the engineered P5HV pathway genes.

TABLE 21
Strains used to produce P5HV from methanol carbon source.

| | Relevant Host | |
|---|---|---|
| Strains | Gene Inactivation | Genes Overexpressed |
| 16 | phaA, phaB, phaC1, | None (control strain) |
| | phaC2, depA, depB | |
| 29 | phaA, phaB, phaC1, | P_{rpsU}-ssaR*; P_{uspA}-phaC3/C1*-orfZ; |
| | phaC2, depA, depB | P_{ompA}-davB-davA-davT |

The strains are grown and evaluated as described in Example 11. The determination of P5HV titers are performed as described in Example 8. Control strain 16 is expected to be unable to produce P5HV, whereas strain 29 is anticipated to produce P5HV owing to the engineered pathway genes.

### Example 18

### Production of P(3HB-Co-5HV) in Methylocystis Hirsuta from Methane

This example shows P(3HB-co-5HV) production from methane as sole carbon source in engineered Methylocystis hirsuta host cells (FIG. 3). The strains used in this example are listed in Table 22. All strains are constructed using the well-known biotechnology tools and methods described above. All strains have the endogenous PHA synthase (phaC1 and phaC2) and depolymerase genes (depA and depB) inactivated, but retain the phaA and phaB genes for copolymer production. Strain 19 lacks all of the recombinant genes, whereas strain 30 contains the engineered P(3HB-co-5HV) pathway genes.

TABLE 22
Strains used to produce P(3HB-co-
5HV) from methanol carbon source.

| | Relevant Host | |
|---|---|---|
| Strains | Gene Inactivation | Genes Overexpressed |
| 19 | phaC1, phaC2, | None (control strain) |
| | depA, depB | |
| 30 | phaC1, phaC2, | P_{rpsU}-ssaR*; P_{uspA}-phaC3/C1*-orfZ; |
| | depA, depB | P_{ompA}-davB-davA-davT |

The strains are grown and evaluated as described in Example 11. The determination of 3HB and 5HV titers of the P(3HB-co-5HV) copolymer are performed as described in Examples 3 and 8. Control strain 19 is expected to be unable to produce P(3HB-co-5HV), whereas strain 30 is anticipated to produce P(3HB-co-5HV) owing to the engineered pathway genes.

### Example 19

### Production of 1,5PD in Methylocystis hirsuta from Methane

This example shows 1,5PD production from methane as sole carbon source in engineered Methylocystis hirsuta host cells (FIG. 3). The strains used in this example are listed in Table 23. All strains are constructed using the well-known biotechnology tools and methods described above. All strains have the endogenous PHB biosynthesis (phaA, phaB, phaC1 and phaC2) and depolymerase genes (depA and depB) inactivated. Strain 16 lacks all of the recombinant genes, whereas strain 31 contains the engineered 1,5PD pathway genes.

TABLE 23
Strains used to produce 1,5-PD from methanol carbon source.

| | Relevant Host | |
|---|---|---|
| Strains | Gene Inactivation | Genes Overexpressed |
| 16 | phaA, phaB, phaC1, | None (control strain) |
| | phaC2, depA, depB | |
| 31 | phaA, phaB, phaC1, | P_{rpsU}-ssaR*; P_{uspA}-pduP-dhaT-orfZ; |
| | phaC2, depA, depB | P_{ompA}-davB-davA-davT |

The strains are grown and evaluated as described in Example 11. 1,5PD in cell culture samples is quantitatively analyzed by GC/MS as described in Example 10. Control strain 16 is expected to be unable to produce 1,5PD, whereas strain 31 is anticipated to produce 1,5PD owing to the engineered pathway genes.

### Example 20

### Generation of Acrylic Acid from Pyrolysis of a Genetically Engineered Biomass Utilizing Methanol to Produce P3HP

In this example, biomass containing P3HP generated as described in Example 1 from genetically engineered Methylophilus methylotrophus using methanol as a feedstock is pyrolyzed in a GC-MS to produce acrylic acid.

To prepare a biomass+P3HP sample for pyrolysis-GC-MS, approximately 20 mL of culture broth was spun down at 6000×g, the cell pellet produced was then washed twice with 0.2% NaCl solution (the solutions were decanted and discarded). The remaining material was frozen at −80° C. for one hour and finally lyophilized over several days to produce a dry biomass+P3HP powder. An Agilent 7890A/5975 GC-MS equipped with a Frontier Lab PY-2020iD pyrolyzer was used to analyze the dried biomass+P3HP (the P3HP was 0.6% by weight). For this technique, a sample is weighed into a steel cup and loaded into a pyrolyzer autosampler. When the pyrolyzer and GC-MS are started for a run, the steel cup is automatically dropped into the pyrolyzer which is set to a specific temperature. The sample is then held in the pyrolyzer for a short period of time while volatiles are released by the sample. The volatiles are then swept using helium gas into the GC column where they condensed onto the column maintained at a temperature of 120° C. Once the pyrolysis is complete, the GC column is heated at a certain rate in order to elute the volatiles released from the sample. The volatile compounds are then swept using helium gas into an electro ionization/mass spectral detector (mass range 10-700 daltons) for identification and quantitation.

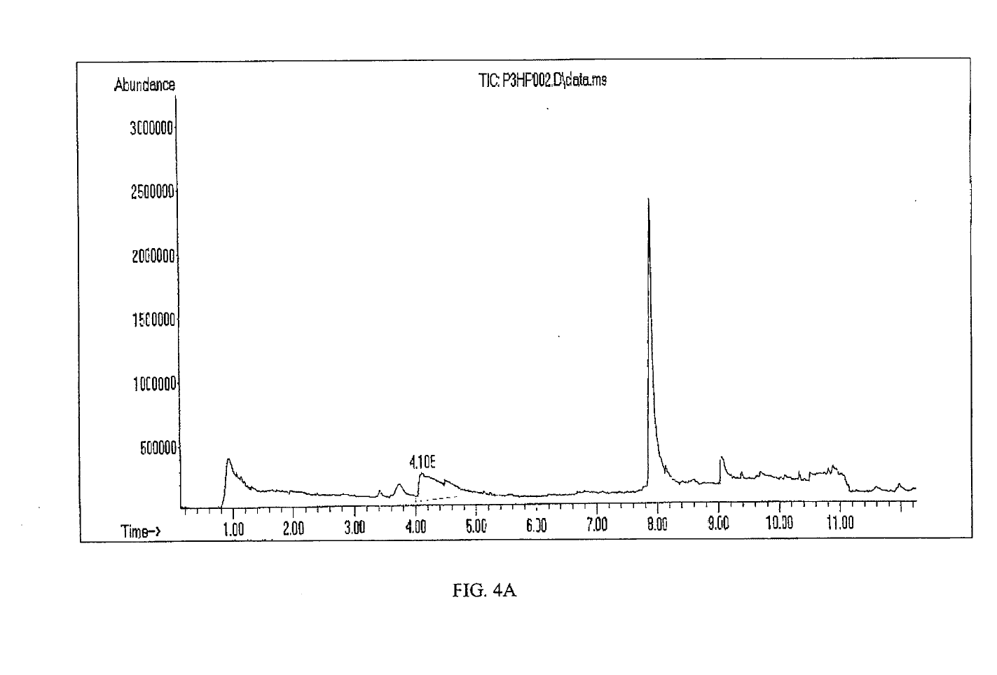

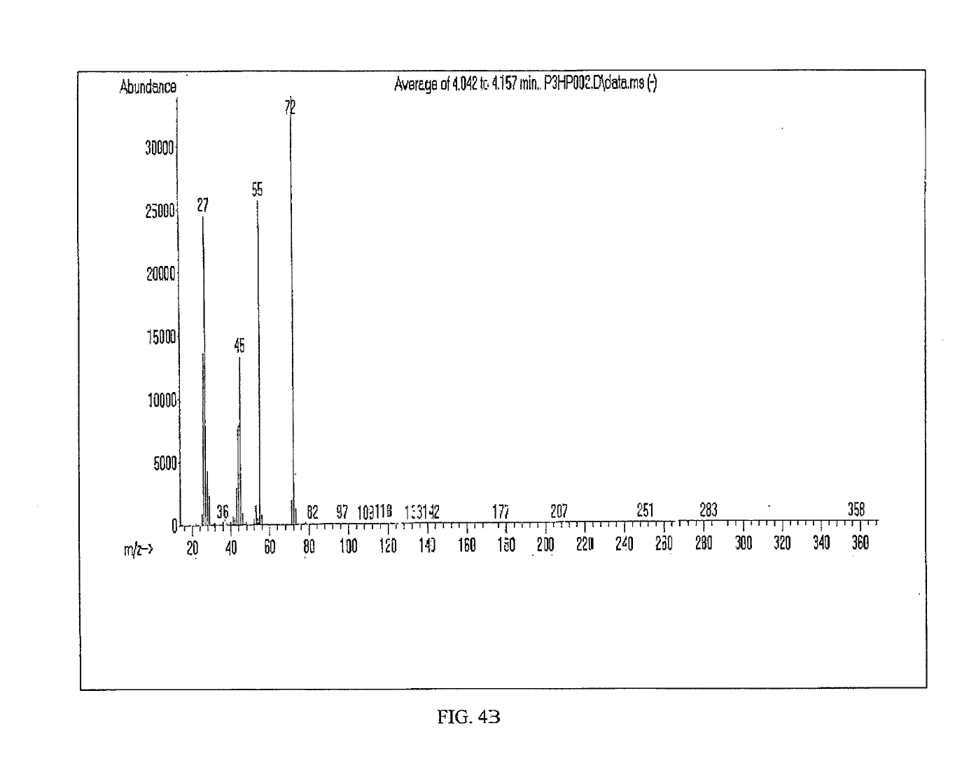

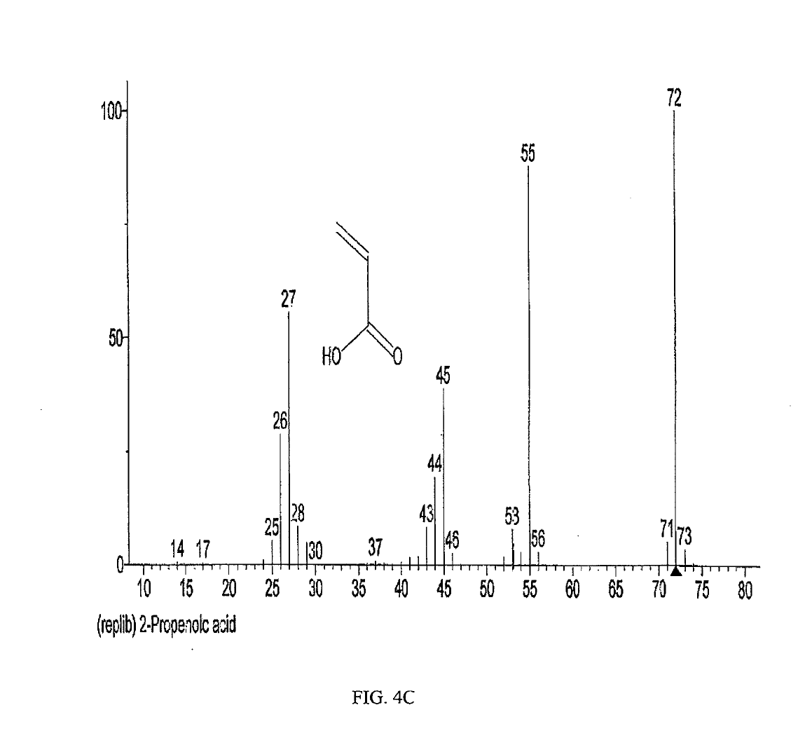

For GC-MS analysis of the dried biomass+P3HP, 1.76 mg of dry biomass was weighed into the steel pyrolyzer cup using a microbalance. The cup was then loaded into the pyrolyzer autosampler and the pyrolyzer programmed to heat to a temperature of 225° C. for a duration of 0.2 minutes. The GC column utilized for separation of the pyrolyzate components was a Hewlett-Packard HP-INNOwax column (length 30 m, ID 0.251 m, film thickness 0.25 μm). The GC oven was programmed to hold at 120° C. for 5 minutes, heat from 120° C. to 240° C. at 10° C./min, then hold for 6 min. Total GC run time was 23 minutes. A split ratio of 50:1 was used during injection of the pyrolyzate vapor onto the GC column. Peaks appearing in the chromatogram plot were identified by the best probability match to spectra from a NIST mass spectral library. The retention time for the acrylic acid (CAS#79-10-7) produced from pyrolysis of P3HP was 4.10-4.12 minutes. FIG. 4 shows the UC-MS chromatogram of the pyrolyzate obtained from the heating of the biomass+P3HP, the mass spectrum of the peak at 4.1-4.2 minutes as well as the spectral library match to this unknown peak. The library match of the mass spectra of the unknown peak at 4.10 minutes showed that this was 2-propenoic acid or acrylic acid with the mass fragments at 27, 45, 55 and 72 m/z.

Gene ID 001 Nucleotide Sequence: Chloroflexus aurantiacus malonyl-CoA reductase (3-hydroxypropionate-forming) mcr_{Ca}*

SEQ ID NO: 16
ATGTCTGGTACTGGTCGACTGGCAGGTAAAATTGCACTGAT
CACTGGCGGTGCTGGCAATATTGGTTCCGAGCTGACCCGCCGTTTCCTGG
CCGAGGGCGCGACCGTCATCATCTCTGGTCGTAACCGCGCCAAACTGAC
CGCACTGGCAGAGCGTATGCAAGCAGAGGCTGGTGTGCCGGCTAAGCGT
ATTGATCTGGAAGTCATGGACGGTAGCGATCCAGTCGCTGTGCGCGCTG
GTATTGAAGCGATTGTGGCTCGCCATGGTCAGATTGATATTCTGGTTAAC
AATGCTGGTTCCGCGGGTGCACAGCGTCGCCTGGCCGAAATTCCGCTGA
CCGAGGCCGAACTGGGTCCGGGCGCTGAGGAAACTCTGCACGCGTCCAT
CGCAAATCTGCTGGGTATGGGCTGGCACCTGATGCGCATTGCGGCTCCA
CACATGCCGGTTGGTTCCGCAGTTATCAACGTTTCCACCATTTTCAGCCG
CGCTGAATACTATGGTCGTATTCCGTACGTTACGCCGAAAGCCGCTCTGA
ACGCGCTGTCCCAGCTGGCGGCACGCGAGCTGGGCGCTCGTGGTATTCG
TGTCAACACTATCTTCCCGGGTCCGATCGAGTCCGACCGTATCCGTACTG
TCTTTCAACGCATGGACCAGCTGAAAGGTCGCCCTGAGGGCGACACCGC
TCATCACTTCCTGAACACCATGCGTCTGTGCCGTGCGAACGATCAGGGCG
CTCTGGAACGTCGCTTCCCGTCCGTGGGTGATGTGGCGGACGCGGCTGTG
TTCCTGGCGTCTGCCGAATCTGCGGCACTGTCTGGTGAGACTATTGAAGT
GACTCACGGCATGGAGCTGCCGGCGTGCTCTGAGACTAGCCTGCTGGCT
CGTACGGATCTGCGCACCATCGACGCTAGCGGTCGCACCACCCTGATCT
GTGCGGGCGACCAGATTGAAGAAGTGATGGCGCTGACCGGTATGCTGCG
TACCTGCGGCTCTGAAGTTATTATCGGCTTCCGCTCCGCAGCAGCGCTGG
CCCAGTTTGAACAGGCGGTCAACGAAAGCCGTCGTCTGGCAGGTGCTGA
TTTTACTCCACCAATCGCCCTGCCGCTGGACCCGCGTGATCCGGCAACTA
TCGATGCTGTGTTTGACTGGGGCGCAGGTGAAAACACCGGCGGCATCCA
CGCTGCTGTTATCCTGCCGGCAACCTCTCATGAGCCAGCCCCTTGTGTGA
TCGAGGTTGATGACGAGCGTGTTCTGAACTTCCTGGCTGACGAGATTACC
GGCACGATCGTTATCGCGTCTCGTCTGGCTCGCTACTGGCAGTCTCAGCG
CCTGACCCCTGGTGCACGTGCCCGTGGCCCTCGTGTTATCTTTCTGTCCA
ATGGCGCGGATCAGAACGGTAACGTCTATGGCCGTATCCAATCTGCTGC
TATCGGCCAACTGATTCGTGTTTGGCGTCACGAAGCTGAGCTGGATTACC
AGCGTGCATCCGCAGCTGGCGATCACGTGCTGCCGCCTGTCTGGGCCAA
CCAAATCGTTCGCTTCGCTAACCGCTCTCTGGAGGGCCTGGAGTTTGCAT
GCGCCTGGACGGCCCAGCTGCTGCACTCTCAGCGTCATATCAATGAAAT
CACTCTGAACATCCCTGCGAACATTAGCGCTACTACCGGTGCTCGTTCTG
CTTCTGTCGGTTGGGCGGAATCTCTGATCGGTCTGCACCTGGGCAAAGTG
GCGCTGATCACCGGTGGCTCTGCGGGCATCGGTGGCCAGATCGGCCGTC
TGCTGGCGCTGTCTGGCGCACGCGTGATGCTGGCTGCACGTGACCGTCAC
AAACTGGAGCAGATGCAGGCAATGATTCAGAGCGAGCTGGCGGAAGTC
GGCTACACTGACGTTGAAGACCGCGTCCACATCGCTCCGGGCTGCGACG
TGTCTTCTGAGGCTCAGCTGGCTGATCTGGTCGAACGCACCCTGTCTGCA
TTCGGTACGGTGGACTACCTGATCAACAATGCGGGCATTGCCGGTGTCG
AGGAGATGGTGATCGACATGCCAGTCGAAGGTTGGCGCCACACGCTGTT
CGCGAATCTGATCAGCAATTACAGCCTGATGCGTAAACTGGCGCCGCTG
ATGAAAAAGCAGGGTTCTGGCTACATCCTGAACGTTTCTTCCTACTTCGG
CGGCGAAAAGGATGCGGCCATCCCATATCCGAACCGCGCAGATTACGCG
GTTTCTAAAGCCGGCCAGCGTGCGATGGCAGAAGTGTTCGCCCGCTTCCT
GGGTCCGGAGATCCAGATTAACGCGATCGCACCGGGTCCGGTTGAAGGT
GATCGCCTGCGTGGTACGGGTGAACGTCCGGGCCTGTTCGCACGTCGTG
CGCGTCTGATCCTGGAAAACAAGCGCCTGAATGAGCTGCACGCGGCCCT
GATTGCAGCCGCGCGTACCGACGAACGTTCTATGCACGAGCTGGTGGAG
CTGCTGCTGCCGAACGATGTGGCTGCCCTGGAACAGAATCCAGCAGCAC
CGACCGCGCTGCGCGAACTGGCCCGTCGTTTTCGTTCCGAAGGCGATCCG
GCTGCATCCTCCTCCAGCGCACTGCTGAACCGTTCTATCGCGGCGAAGCT
GCTGGCACGCCTGCACAATGGTGGTTACGTCCTGCCAGCCGACATCTTCG
CAAACCTGCCTAACCCACCGGATCCATTCTTTACCCGCGCTCAGATCGAC
CGTGAAGCGCGTAAAGTTCGTGATGGTATCATGGGCATGCTGTATCTGC
AGCGTATGCCGACGGAGTTCGATGTCGCGATGGCAACCGTCTATTACCT
GGCCGACCGCAACGTGAGCGGCGAAACCTTCCACCCATCCGGTGGCCTG
CGCTATGAACGTACGCCGACCGGTGGTGAGCTGTTCGGCCTGCCGAGCC
CGGAACGCCTGGCAGAACTGOTTGGCTCCACCGTGTACCTGATCGGTGA
ACACCTGACGGAGCACCTGAACCTGCTGGCCCGTGCGTATCTGGAGCGT
TATGGCGCACGTCAAGTTGTTATGATCGTGGAAACCGAAACGGGTGCCG
AAACTATGCGTCGTCTGCTGCACGACCATGTCGAAGCCGGCCGCCTGAT
GACGATCGTGGCTGGTGACCAGATCGAAGCAGCCATCGATCAGGCAATT
ACGCGTTATGGTCGTCCGGGTCCTGTTGTTTGCACTCCATTCCGCCCGCT
GCCAACTGTGCCTCTGGTCGGTCGCAAGGACTCCGATTGGAGCACGGTC
CTGTCTGAAGCTGAGTTCGCGGAACTGTGCGAGCATCAGCTGACTCACC
ACTTCCGTGTTGCTCGCAAGATCGCACTGTCCGATGGCGCCAGCCTGGCG
CTGGTCACCCCAGAGACTACCGCAACTTCTACCACTGAACAATTCGCTCT
GGCAAACTTCATTAAAACTACGCTGCACGCTTTCACCGCGACCATCGGC
GTTGAGTCCGAACGTACGGCGCAGCGTATCCTGATCAATCAGGTGGATC
TGACTCGTCGTGCGCGCOCCGAAGAACCGCGCGATCCGCACGAACGCCA
GCAGGAACTGGAGCGCTTCATTGAAGCAGTCCTGCTGGTCACTGCGCCT
CTGCCACCGGAAGCGGACACGCGCTATGCGGTCGCATCCATCGCGGCC
GTGCCATCACTGTCTGA,

Gene ID 001 Protein Sequence: Chloroflexus aurantiacus malonyl-CoA reductase (3-hydroxypropionate-forming) Mcr_{Ca}*

SEQ ID NO: 17
MSGTGRLAGKIALITGGAGNIGSELTRRFLAEGATVIISGRNRA
KLTALAERMQAEAGVPAKRIDLEVMDGSDPVAVRAGIEAIVARHGQIDIL
VNNAGSAGAQRRLAEIPLTEAELGPGAEETLHASIANLLGMGWHLMRIAA
PHMPVGSAVINVSTIFSRAEYYGRIPYVTPKAALNALSQLAARELGARGI
RVNTIFPGPIESDRIRTVFQRMDQLKGRPEGDTAHHFLNTMRLCRANDQG
ALERRFPSVCIDVADAAVFLASAESAALSGETIEVTHGMELPACSETSLL
ARTDLRTIDASGRTTLICAGDQIEEVMALTGMLRTCGSEVIIGFRSAAAL
AQFEQAVNESRRLAGADFTPPIALPLDPRDPATIDAVFDWGAGENTGGIH
AAVILPATSHEPAPCVIEVDDERVLNFLADEITGTIVIASRLARYWQSQR
LTPGARARGPRVIFLSNGADQNGNVYGRIQSAAIGQLIRVWRHEAELDYQ
RASAAGDHVLPPVWANQIVRFANRSLEGLEFACAWTAQLLHSQRHINEIT
LNIPANISATTGARSASVGWAESLIGLHLGKVALITGGSAGIGGQIGRLL
ALSGARVMLAARDRHKLEQMQAMIQSELAEVGYTDVEDRVHIAPGCDVSS
EAQLADLVERTLSAFGTVDYLINNAGIAGVEEMVIDMPVEGWRHTLFANL
ISNYSLMRKLAPLMKKQGSGYILNVSSYFGGEKDAAIPYPNRADYAVSKA
GQRAMAEVFARFLGPEIQINAIAPGPVEGDRLRGTGERPGLFARRARLIL
ENKRLNELHAALIAAARTDERSMHELVELLLPNDVAALEQNPAAPTALRE
LARRFRSEGDPAASSSSALLNRSIAAKIIARLHNGGYVLPADIFANLPNP
PDPFFTRAQIDREARKVRDGIMGMLYLQRMPTEFDVAMATVYYLADRNVS
GETFHPSGGLRYERTPTGGELFGLPSPERLAELVGSTVYLIGEHLTEHLN
LLARAYLERYGARQVVMIVETETGAETMRRLLHDHVEAGRLMTIVAGDQI
EAAIDQAITRYGRPGPVVCTPFRPLPTVPLVGRKDSDWSTVLSEAEFAEL
CEHQLTHHFRVARKIALSDGASLALVTPETTATSTTEQFALANFIKTTLH
AFTATIGVESERTAQRILINQVDLTRRARAEEPRDPHERQQELERFIEAV
LLVTAPLPPEADTRYAGRIHRGRAITV,

1-111. (canceled)

112. A method of producing a polymer product, the method comprising:
feeding a genetically engineered methylotroph with a renewable feedstock comprising methane or methanol as the sole carbon source, the genetically engineered methylotroph producing the polymer product selected from a homopolymer or a copolymer of a 4-carbon (C4) monomer or a homopolymer or a copolymer of a 5-carbon (C5) monomer,
wherein the genetically engineered methylotroph is genetically modified to stably express one or more genes that encode one or more enzymes of the C4 or C5 pathway.

113-115. (canceled)

116. The method of claim 112, wherein the renewable feedstock is methanol.

117. The method of claim 112, wherein the renewable feedstock is methane.

118-125. (canceled)

126. The method of claim 112, wherein the polymer product is poly-3-hydroxybutyrate-co-3-hydroxyproprionate copolymer and the genetically engineered methylotroph is genetically engineered to modify a pathway selected from a malonyl-CoA reductase metabolic pathway, and a dihydroxyacetone-phosphate metabolic pathway.

127. The method of claim 126, wherein the genetically engineered methylotroph is genetically engineered to modify the malonyl-CoA reductase metabolic pathway, and wherein the one or more genes that are stably expressed encode one or more enzyme selected from acetyl-CoA acetyltransferase; acetoacetyl-CoA reductase; acetyl-CoA carboxylase, malonyl-CoA reductase (3-hydroxypropionate-forming), malonyl-CoA reductase (malonate semialdehyde-forming), malonic semialdehyde reductase, CoA transferase, CoA ligase, and polyhydroxyalkanoate synthase, wherein the expression increases the production of poly-3-hydroxybutyrate-co-3-hydroxyproprionate copolymer.

128. The method of claim 126, wherein the genetically engineered methylotroph is genetically engineered to modify the malonyl-CoA reductase metabolic pathway, and wherein the one or more genes that are stably expressed encode one or more enzyme selected from:
acetyl-CoA acetyltransferase from Zoogloea ramigera; acetoacetyl-CoA reductase from Zoogloea ramigera; an acetyl-CoA carboxylase subunits from E. coli; a malonyl-CoA reductase (3-hydroxypropionate-forming) from Chloroflexus aurantiacus; malonyl-CoA reductase (malonate semialdehyde-forming) from Sulfolobus tokodaii str. 7; malonic semialdehyde reductase from Sulfolobus tokodaii str. 7; CoA transferase from Clostridium kluyveri DSM 555, CoA ligase from Pseudomonas putida; and polyhydroxyalkanoate synthase from a fusion protein of Pseudomonas putida and Ralstonia eutropha JMP134; wherein the expression increases the production of poly-3-hydroxybutyrate-co-3-hydroxyproprionate copolymer.

129. The method of claim 126, wherein the genetically engineered methylotroph is Methylophilus methylotrophus, Methylobacterium extorquens with one or more of the following genes deleted: phaC1, phaC2, depA and depB, or Methylocystis hirsute having one or more of the following genes deleted: phaC1, phaC2, depA and depB.

130-131. (canceled)

132. The method of claim 126, wherein the genetically engineered methylotroph is genetically engineered to modify the dihydroxyacetone-phosphate metabolic pathway, and wherein the one or more genes that are stably expressed encode one or more enzymes selected from: glycerol-3-phosphate dehydrogenase (NAD+); glycerol-3-phosphate dehydrogenase (NADP+); glycerol-3-phosphatase; glycerol dehydratase; glycerol dehydratase reactivating enzyme; aldehyde dehydrogenase; alcohol dehydrogenase; aldehyde reductase, acetyl-CoA acetyltransferase; acetoacetyl-CoA reductase; CoA-acylating 3-hydroxypropionaldehyde dehydrogenase; and polyhydroxyalkanoate synthase,
wherein the expression increases the production of poly-3-hydroxybutyrate-co-3-hydroxyproprionate copolymer.

133. The method of claim 126, wherein the genetically engineered methylotroph is genetically engineered to modify the dihydroxyacetone-phosphate metabolic pathway, and wherein the one or more genes that are stably expressed encode one or more enzyme selected from glycerol-3-phosphate dehydrogenase (NAD+) from Saccharomyces cerevisiae S288c; glycerol-3-phosphate dehydrogenase (NADP+) from Rickettsia prowazekii (strain Madrid E); glycerol-3-phosphatase from Saccharomyces cerevisiae S288c; glycerol dehydratase small, medium and large subunits from Klebsiella pneumonia; glycerol dehydratase reactivating enzyme (Chain A and Chain B) from Klebsiella pneumonia; 3-hydroxy-propionaldehyde dehydrogenase (gamma-Glu-gamma-aminobutyraldehyde dehydrogenase, NAD(P)H-dependent) from E. coli str. K-12 substr. MG1655; and aldehyde reductase (succinic semialdehyde reductase) from E. coli K-12; acetyl-CoA acetyltransferase from Zoogloea ramigera; acetoacetyl-CoA reductase from Zoogloea ramigera; aldehyde dehydrogenase/alcohol dehydrogenase from E. coli str. K-12 substr. MG1655; CoA-acylating 3-hydroxypropionaldehyde dehydrogenase from Salmonella enterica subsp. enterica serovar Typhimurium str. LT2; and polyhydroxyalkanoate synthase from a fusion protein of Pseudomonas putida and Ralstonia eutropha JMP134,
wherein the expression increases the production of poly-3-hydroxybutyrate-co-3-hydroxyproprionate copolymer.

134-143. (canceled)

144. The method of claim 112, wherein the polymer product is poly-4-hydroxybutyrate and wherein the genetically engineered methylotroph is genetically engineered to modify a succinate semialdehyde dehydrogenase pathway, and, optionally, an alpha-ketoglutarate decarboxylase pathway.

145. The method of claim 144, wherein the one or more genes that are stably expressed encode one or more enzymes selected from: succinate semialdehyde dehydrogenase, alpha-ketoglutarate decarboxylase, succinic semialdehyde reductase, CoA transferase, CoA ligase, butyrate kinase, phosphotransbutyrylase, 4-hydroxybutyryl-CoA reductase and 4-hydroxybutyrylaldehyde reductase; wherein the expression increases the production of poly-4-hydroxybutyrate.

146. The method of claim 144, wherein the genetically engineered methylotroph is Methylophilus methylotrophus or Methylocystis hirsute having one or more of the following genes deleted: pha A, phaB, phaC1, phaC2, depA and depB.

147. The method of claim 112, wherein the polymer product is poly-3-hydroxybutyrate-co-4-hydroxybutyrate and the genetically engineered methylotroph is genetically engineered to modify a succinate semialdehyde dehydrogenase pathway, and, optionally, an alpha-ketoglutarate decarboxylase pathway or a crotonase pathway.

148. The method of claim 147, wherein the one or more genes that are stably expressed encode one or more enzymes selected from: acetyl-CoA acetyltransferase; acetoacetyl-CoA reductase; succinate semialdehyde dehydrogenase, alpha-ketoglutarate decarboxylase, succinic semialdehyde reductase, CoA transferase, CoA ligase, butyrate kinase, phosphotransbutyrylase, 4-hydroxybutyryl-CoA reductase; 4-hydroxybutyrylaldehyde reductase; acetyl-CoA transferase and acetoacetyl-CoA reductase; crotonase; and polyhydroxyalkanoate synthase, wherein the expression increases the production of poly-3-hydroxybutyrate-co-4-hydroxybutyrate.

149. The method of claim 147, wherein the genetically engineered methylotroph is Methylophilus methylotrophus or Methylobacterium extorquens having one or more of the following genes deleted: phaC1, phaC2, depA and depB, or Methylocystis hirsute having one or more of the following genes deleted: phaC1, phaC2, depA and depB.

150-152. (canceled)

153. The method of claim 112, wherein the polymer product is poly-5-hydroxyvalerate and the pathway is a lysine pathway.

154. The method of claim 153, wherein the one or more genes that are stably expressed encode one or more enzymes selected from lysine 2-monooxygenase, 5-aminopentanamidase; aminopentanoate transaminase; succinate semialdehyde reductase; CoA-transferase; Co-A ligase; and polyhroxyalkanoate synthase; wherein the expression increases the production of poly-5-hydroxyvalerate.

155. The method of claim 154, wherein the genetically modified methylotroph is Methylophilus methylotrophus, Methylocystis hirsute having one or more of the following genes deleted: pha A, phaB, phaC1, phaC2, depA and depB.

156. The method of claim 112, wherein the polymer product is poly-3-hydroxybutyrate-co-5-hydroxyvalerate and wherein the genetically engineered methylotroph is genetically engineered to modify a lysine pathway.

157. The method of claim 156, wherein the one or more genes that are stably expressed encode one or more enzymes selected from acetyl-CoA acetyltransferase; acetoacetyl-CoA reductase; polyhydroxyalkanoate synthase; lysine 2-monooxygenase, 5-aminopentanamidase; aminopentanoate transaminase; succinate semialdehyde reductase; CoA-transferase; Co-A; and polyhydroxyalkanoate synthase; wherein the expression increases the production of poly-3-hydroxybutyrate-co-5-hydroxyvalerate copolymer.

158. The method of claim 156, wherein the genetically engineered methylotroph is Methylophilus methylotrophus, Methylobacterium extorquens, or Methylocystis hirsute having one or more of the following genes deleted: phaC1, phaC2, depA and depB.

159-193. (canceled)

194. The method of claim 148, wherein the one or more genes that are stably expressed encode polyhydroxyalkanoate synthase from a fusion protein of Pseudomonas putida and Ralstonia eutropha JMP134.

195-207. (canceled)

208. The method of claim 157, wherein the one or more genes that are stably expressed encode one or more enzymes selected from acetyl-CoA acetyltransferase from Zoogloea ramigera, acetoacetyl-CoA reductase from Zoogloea ramigera, and polyhydroxyalkanoate synthase from a fusion protein of Pseudomonas putida and Ralstonia eutropha JMP134.

209-212. (canceled)

213. The method of claim 112, wherein the method further includes culturing a genetically engineered organism with a renewable feedstock to produce a biomass.

214-219. (canceled)

220. The method of claim 112, wherein the genetically engineered methylotroph is selected from: Methylophilus methylotrophus AS-1; Methylocystis hirsute; Methylophilus methylotrophus M12-4, Methylophilus methylotrophus M1, Methylophilus methylotrophus sp. (deposited at NCIMB as Acc. No. 11809), Methylophilus leisingeri, Methylophilus flavus sp. nov., Methylophilus luteus sp. nov., Methylomonas sp. strain 16a, Methylomonas methanica MC09, Methylobacterium extorquens AM1 (formerly known as Pseudomonas AM1), Methylococcus capsulatus Bath, Methylomonas sp. strain J, Methylomonas aurantiaca, Methylomonas fodinarum, Methylomonas scandinavica, Methylomonas rubra, Methylomonas streptobacterium, Methylomonas rubrum, Methylomonas rosaceous, Methylobacter chroococcum, Methylobacter bovis, Methylobacter capsulatus, Methylobacter vinelandii, Methylococcus minimus, Methylosinus sporium, Methylocystis parvus, Methylocystis hirsute, Methylobacterium organophilum, Methylobacterium rhodesianum, Methylobacterium R6, Methylobacterium aminovorans, Methylobacterium chloromethanicum, Methylobacterium dichloromethanicum, Methylobacterium fujisawaense, Methylobacterium mesophilicum, Methylobacterium radiotolerans, Methylobacterium rhodinum, Methylobacterium thiocyanatum, Methylobacterium zatmanii, Methylomonas methanica, Methylomonas albus, Methylomonas agile, Methylomonas P11, Methylobacillus glycogenes, Methylosinus trichosporium, Hyphomicrobium methylovorum, Hyphomicrobium zavarzinii, Bacillus methanolicus, Bacillus cereus M-33-1, Streptomyces 239, Mycobacterium vaccae, Diplococcus PAR, Protaminobacter ruber, Rhodopseudomonas acidophila, Arthrobacter rufescens, Arthrobacter 1A1 and 1A2, Arthrobacter 2B2, Arthrobacter globiformis SK-200, Klebsiella 101, Pseudomonas 135, Pseudomonas oleovorans, Pseudomonas rosea (NCIB 10597 to 10612), Pseudomonas extorquens (NCIB 9399), Pseudomonas PRL-W4, Pseudomonas AM1 (NCIB 9133), Pseudomonas AM2, Pseudomonas M27, Pseudomonas PP, Pseudomonas 3A2, Pseudomonas RJ1, Pseudomonas TP1, Pseudomonas sp. 1 and 135, Pseudomonas sp. YR, JB1 and PCTN, Pseudomonas methylica sp. 2 and 15, Pseudomonas 2941, Pseudomonas AT2, Pseudomonas 80, Pseudomonas aminovorans, Pseudomonas sp. 1A3, 1B1, 7B1 and 8B1, Pseudomonas S25, Pseudomonas (methylica) 20, Pseudomonas W1, Pseudomonas W6 (MB53), Pseudomonas C, Pseudomonas MA, Pseudomonas MS. Exemplary yeast strains include: Pichia pastoris, Gliocladium deliquescens, Paecilomyces varioti, Trichoderma lignorum, Hansenula polymorpha DL-1 (ATCC 26012), Hansenula polymorpha (CBS 4732), Hansenula capsulata (CBS 1993), Hansenula lycozyma (CBS 5766), Hansenula henricii (CBS 5765), Hansenula minuta (CBS 1708), Hansenula nonfermentans (CBS 5764), Hansenula philodenda (CBS), Hansenula wickerhamii (CBS 4307), Hansenula ofuaensis, Candida boidinii (ATCC 32195), Candida boidinii (CBS 2428, 2429), Candida boidinii KM-2, Candida boidinii NRRL Y-2332, Candida boidinii S-1, Candida boidinii S-2, Candida boidinii 25-A, Candida alcamigas, Candida methanolica, Candida parapsilosis, Candida utilis (ATCC 26387), Candida sp. N-16 and N-17, Kloeckera sp. 2201, Kloeckera sp. A2, Pichia pinus (CBS 5098), Pichia pinus (CBS 744), Pichia pinus NRRL YB-4025, Pichia haplophila (CBS 2028), Pichia pastoris (CBS 704), Pichia pastoris (IFP 206), Pichia trehalophila (CBS 5361), Pichia lidnerii, Pichia methanolica, Pichia methanothermo, Pichia sp. NRRL-Y-11328, Saccharomyces H-1, Torulopsis pinus (CBS 970), Torulopsis nitatophila (CBS 2027), Torulopsis nemodendra (CBS 6280), Torulopsis molishiana, Torulopsis methanolovescens, Torulopsis glabrata, Torulopsis enoki, Torulopsis methanophiles, Torulopsis methanosorbosa, Torulopsis methanodomercquii, Torulopsis nagoyaensis, Torulopsis sp. A1, Rhodotorula sp., Rhodotorula glutinis (strain cy), and Sporobolomyces roseus (strain y).
